(19) United States
(12) Patent Application Publication
(10) Pub. No.: US 2026/0124135 A1
(43) Pub. Date: May 7, 2026

(54) IMPLANTABLE SCAFFOLDS AND METHODS OF USE

(71) Applicants: North Carolina State University, Raleigh, NC (US); The University of North Carolina at Chapel Hill, Chapel Hill, NC (US)

(72) Inventors: Yevgeny Brudno, Raleigh, NC (US); Madelyn VanBlunk, Raleigh, NC (US); Sharda Pandit, Raleigh, NC (US); Pritha Agarwalla, Raleigh, NC (US); Gianpietro Dotti, Chapel Hill, NC (US)

(21) Appl. No.: 19/120,162

(22) PCT Filed: Oct. 13, 2023
(86) PCT No.: PCT/US2023/076867

§ 371 Date: Apr. 10, 2025

Related U.S. Application Data
(60) Provisional application No. 63/415,806, filed on Oct. 13, 2022.
(60) Provisional application No. 63/441,409, filed on Jan. 26, 2023.

(51) Int. Cl. A61K 9/00; A61K 38/19; A61K 38/20; A61K 38/21; A61K 40/11; A61K 40/13; A61K 40/15; A61K 40/17; A61K 40/19; A61K 47/36; A61K 48/00; A61P 35/00

(52) U.S. Cl. CPC A61K 9/0024; A61K 38/191; A61K 38/20; A61K 38/2013; A61K 38/2086; A61K 38/217; A61K 40/11; A61K 40/13; A61K 40/15; A61K 40/17; A61K 40/19; A61K 47/36; A61K 48/0033; A61P 35/00

(57) ABSTRACT

The present disclosure provides compositions, systems, and methods related to cellular transduction. In particular, the present disclosure provides compositions, systems, and methods pertaining to implantable macroporous scaffolds that facilitate rapid and highly efficient cellular transduction.

### CROSS REFERENCE TO RELATED APPLICATIONS

This application claims priority to and the benefit of U.S. Provisional Patent Application No. 63/415,806 filed Oct. 13, 2022, and U.S. Provisional Patent Application No. 63/441,409 filed Jan. 26, 2023, both of which are incorporated herein by reference in their entireties and for all purposes.

### GOVERNMENT SUPPORT

This invention was made with government support under grant number CA260223 awarded by the National Institutes of Health. The government has certain rights in the invention.

### FIELD

The present disclosure provides compositions, systems, and methods related to cellular transduction. In particular, the present disclosure provides compositions, systems, and methods pertaining to implantable macroporous scaffolds that facilitate rapid and highly efficient cellular transduction.

### BACKGROUND

Chimeric Antigen Receptor (CAR) T cell therapy has had revolutionary clinical success in hematological cancers and demonstrated potential against a wide array of cancer types. Unfortunately, CAR T cell therapy has not to date had the same impact in solid tumors. This challenge stems from several limitations of current CAR T cell products, including extensive manufacturing procedures, therapeutic toxicity, limited in vivo persistence, and inability to achieve long term therapeutic efficacy. The costly and labor-intensive CAR T cell manufacturing presents several challenges for the success of these therapies across various hematological and solid malignancies. Current CAR T cell manufacturing requires extensive infrastructure and takes multiple weeks for vein-to-vein procedures, delaying the initiation of the treatment in patients with already advancing disease. These long manufacturing times (>2 weeks) and high treatment costs (˜$350K) limit widespread use of this therapy. Approaches to simplify this process include automated manufacturing, use of allogeneic products, and rapid CAR T cell manufacturing eliminating one or more CAR T cells generation steps. However, these approaches are limited by poor quality products, life-threatening toxicities, low persistence, and precocious cell differentiation and exhaustion. Generating and expanding tumor-specific CAR T cells in vivo could mitigate some of these problems by reducing manufacturing time, treatment cost, and producing long lasting highly efficacious cell products.

In vivo expansion and long-term persistence of infused CAR T cells is critical to provide antitumor function and prevent tumor relapse. The importance of T cell persistence and long-term function is particularly acute in solid tumors, where physiological and immunological barriers such as hypoxia and the immunosuppressive tumor microenvironment alter CAR T cell metabolism and promote CAR T cell exhaustion leading to limited therapeutic outcomes. Recent work suggests that less differentiated CAR T cells could increase cell engraftment and persistence, leading to improved outcomes. Strategies to produce less differentiated CAR T cell phenotypes show promise and include an initial selection of naïve cells, optimization of in vitro culture time and cytokine regiments as well as addition of small molecule T cell regulators. Despite extensive studies, initial selection of naïve population or maintaining naïve population during clinical-grade manufacturing of CAR T cells is associated with multiple technical difficulties. A simple, scalable, and tunable CAR T cell manufacturing platform to generate CAR T cells with less differentiated phenotypes would improve long-term persistence and therapeutic efficacy.

Biomaterials could overcome many of the obstacles to widespread, safe, and efficacious CAR T cell therapy for solid tumors by providing a nurturing niche to optimize cell development. Recent efforts have used biomaterials to improve specific, individual steps of CAR T cell manufacturing, including in vitro and in vivo T cell isolation, T cell activation, genetic modification, expansion, and delivery. In addition, biomaterials were used to control T cell proliferation and differentiation to produce robust antitumor effects. Finally, biomaterials enable sustained release of CAR T cells, which could improve the safety and toxicity profile of CAR T cell therapy. Recent work suggests that splitting CAR T cell administration into multiple, smaller doses can lower the toxic profile and expand the therapeutic window of this therapy. Sustained release enables an improved multi-dose schedule creating an avenue for significant CAR T cell doses without an initial toxic burst of effector function.

### SUMMARY

Embodiments of the present disclosure include an implantable macroporous scaffold comprising a crosslinked biopolymer matrix comprising an average pore size ranging from about 10 μm to about 500 μm, and a stiffness ranging from about 1 kPa to about 1000 kPa, wherein the stiffness of the matrix is compatible with the stiffness of a target tissue; and a composition comprising a plurality of cells and a transduction agent. In accordance with these embodiments, the scaffold facilitates transduction of the plurality of cells with the transduction agent.

In some embodiments, the biopolymer matrix comprises at least one of alginate, Hyaluronic acid, collagen, fibrin, Poly Lactic-co-Glycolic Acid (PLGA), Polycaprolactone (PCL), gelatin, Polyethylene glycol (PEG), chitosan, cellulose, polyglutamic acid, fibrin, silk, agarose, dextran, polyacrylamide, polyvinyl alcohol, Poly(N-isopropylacrylamide), Poly(2-hydroxyethyl methacrylate), polyurethane, polyethyleneimine, Poly(methyl methacrylate, Poly(2-oxazoline), Polyphosphazenes, and any composites, derivatives, or combinations thereof.

In some embodiments, the biopolymer matrix comprises alginate having a molecular weight from about 1 kDa to about 500 kDa.

In some embodiments, the biopolymer matrix comprises alginate having a G/M ratio from about 0.5 to about 5.0.

In some embodiments, the biopolymer matrix comprises alginate at a concentration ranging from about 0.1% to about 5.0%.

In some embodiments, the biopolymer matrix comprises calcium alginate having a calcium concentration ranging from about 0.1% to about 1.0%.

In some embodiments, the biopolymer matrix is generated at a temperature ranging from about 0° C. to about −80° C.

In some embodiments, the biopolymer matrix exhibits a stiffness that is from about ±25%, about ±50%, about ±75%, about ±100%, about ±125%, about ±150%, about ±175%, about ±200%, about ±225%, or about ±250% of the stiffness of the target tissue.

In some embodiments, the scaffold comprises at least one biological agent. In some embodiments, the biological agent is a small molecule. In some embodiments, the small molecule is selected from the group consisting of a TLR agonist, a checkpoint inhibitor, an IDO inhibitor, a MEK inhibitor, an HDAC inhibitor, a PI3K inhibitor, an immunomodulatory drug, a JAK kinase inhibitor, and an mTOR inhibitor.

In some embodiments, the at least one biological agent is a protein, peptide, or polypeptide. In some embodiments, the protein, peptide, or polypeptide is selected from the group consisting of a cytokine, an antibody, and a growth factor. In some embodiments, the cytokine comprises at least one of IL-2, IL-15, IL-7, IL-23, TNF-α, and/or IFN-γ.

In some embodiments, the plurality of cells comprise one or more immune cells. In some embodiments, the one or more immune cells are selected from the group consisting of a T cell, a B cell, a natural killer (NK) cell, an NK T cell, a macrophage, a dendritic cell, a tumor infiltrating lymphocyte (TIL), a tumor infiltrating NK cell (TINK), and a marrow infiltrating lymphocyte (MIL). In some embodiments, the one or more immune cells are activated.

In some embodiments, the plurality of cells are obtained from cell culture. In some embodiments, the plurality of cells are obtained from a donor.

In some embodiments, the transduction agent comprises a viral vector. In some embodiments, the viral vector is selected from the group consisting of a lentivirus, a retrovirus, an adenovirus, an adeno-associated virus, a cocal virus, and a baculovirus.

In some embodiments, the transduction agent comprises a virus-like particle, a cell-mimicking particle, a transposon, an exosome, a nanoparticle, a micelle, and a liposome.

In some embodiments, the transduction agent comprises a nucleic acid cargo. In some embodiments, the nucleic acid cargo comprises siRNA, tasiRNA, lncRNA, shRNA, mRNA, gRNA, miRNA, and/or viral RNA. In some embodiments, the nucleic acid cargo comprises DNA that encodes a fusion protein, a chimeric antigen receptor (CAR), a therapeutic peptide or polypeptide, or a combination thereof.

In some embodiments, the scaffold is implanted within or adjacent to the target tissue. In some embodiments, the target tissue is tumor tissue. In some embodiments, the target tissue is solid tumor tissue. In some embodiments, the target tissue comprises at least one of lung tissue, bone tissue, skin tissue, breast tissue, muscle tissue, nerve tissue, brain tissue, lymph tissue, prostate tissue, bladder tissue, stomach tissue, intestinal tissue, uterine tissue, ovarian tissue, liver tissue, adipose tissue, cartilaginous tissue, thyroid tissue, and/or pancreatic tissue.

In some embodiments, the scaffold facilitates the transduction of the plurality of cells with the transduction agent with a transduction efficiency of at least 50%.

In some embodiments, the macroporous scaffolds of the present disclosure comprise a biopolymer matrix having an average pore size ranging from about 50 μm to about 250 μm. In some embodiments, the macroporous scaffolds of the present disclosure comprise a biopolymer matrix having an average pore size ranging from about 100 μm to about 200 μm. In some embodiments, the macroporous scaffolds of the present disclosure comprise a biopolymer matrix having an average pore size ranging from about 50 μm to about 150 μm.

Embodiments of the present disclosure also include a method of treating a subject. In accordance with these embodiments, the method includes, implanting a macroporous scaffold within or adjacent to a target tissue, wherein the scaffold comprises: a crosslinked biopolymer matrix comprising an average pore size ranging from about 10 μm to about 500 μm, and a stiffness ranging from about 1 kPa to about 1000 kPa, wherein the stiffness of the matrix is compatible with the stiffness of the target tissue; and a composition comprising a plurality of cells and a transduction agent. In some embodiments, the scaffold facilitates transduction of the plurality of cells with the transduction agent, and wherein the transduced cells treat the subject.

In some embodiments of the method, target tissue is tumor tissue. In some embodiments of the method, the target tissue is solid tumor tissue. In some embodiments of the method, the target tissue comprises at least one of lung tissue, bone tissue, skin tissue, breast tissue, muscle tissue, nerve tissue, brain tissue, lymph tissue, prostate tissue, bladder tissue, stomach tissue, intestinal tissue, uterine tissue, ovarian tissue, liver tissue, adipose tissue, cartilaginous tissue, thyroid tissue, and/or pancreatic tissue.

In some embodiments of the method, the subject has been diagnosed with a disease or condition. In some embodiments of the method, the disease or condition comprises cancer.

In some embodiments of the method, the scaffold facilitates the transduction of the plurality of cells with the transduction agent with a transduction efficiency of at least 50%.

In some embodiments of the method, the biopolymer matrix comprises at least one of alginate, Hyaluronic acid, collagen, fibrin, Poly Lactic-co-Glycolic Acid (PLGA), Polycaprolactone (PCL), gelatin, Polyethylene glycol (PEG), chitosan, cellulose, polyglutamic acid, fibrin, silk, agarose, dextran, polyacrylamide, polyvinyl Poly(N-isopropylacrylamide), Poly(2-hydroxyethyl methacrylate), polyurethane, polyethyleneimine, Poly(methyl methacrylate, Poly(2-oxazoline), Polyphosphazenes, and any composites, derivatives, or combinations thereof.

In some embodiments of the method, the biopolymer matrix comprises alginate having a molecular weight from about 1 kDa to about 500 kDa. In some embodiments of the method, the biopolymer matrix comprises alginate having a G/M ratio from about 0.5 to about 5.0. In some embodiments of the method, the biopolymer matrix comprises alginate at a concentration ranging from about 0.1% to about 5.0%. In some embodiments of the method, the biopolymer matrix comprises calcium alginate having a calcium concentration ranging from about 0.1% to about 1.0%. In some embodiments of the method, the biopolymer matrix is generated at a temperature ranging from about −20° C. to about −80° C.

In some embodiments of the method, the biopolymer matrix exhibits a stiffness that is from about ±25%, about ±50%, about ±75%, about ±100%, about ±125%, about ±150%, about ±175%, about ±200%, about ±225%, or about ±250% of the stiffness of the target tissue.

In some embodiments of the method, the scaffold comprises at least one biological agent. In some embodiments of the method, the at least one biological agent is a small molecule. In some embodiments of the method, the small molecule is selected from the group consisting of a TLR agonist, a checkpoint inhibitor, an IDO inhibitor, a MEK inhibitor, an HDAC inhibitor, a PI3K inhibitor, an immunomodulatory drug, a JAK kinase inhibitor, and an mTOR inhibitor. In some embodiments of the method, the at least one biological agent is a protein, peptide, or polypeptide. In some embodiments of the method, the protein, peptide, or polypeptide is selected from the group consisting of a cytokine, an antibody, and a growth factor. In some embodiments of the method, the cytokine comprises at least one of IL-2, IL-15, IL-7, IL-23, TNF-α, and/or IFN-γ.

In some embodiments of the method, the plurality of cells comprise one or more immune cells. In some embodiments of the method, the one or more immune cells are selected from the group consisting of a T cell, a B cell, a natural killer (NK) cell, an NK T cell, a macrophage, a dendritic cell, a tumor infiltrating lymphocyte (TIL), a tumor infiltrating NK cell (TINK), and a marrow infiltrating lymphocyte (MIL). In some embodiments of the method, the one or more immune cells are activated. In some embodiments of the method, the plurality of cells are obtained from cell culture. In some embodiments of the method, the plurality of cells are obtained from a donor.

In some embodiments of the method, the transduction agent comprises a viral vector. In some embodiments of the method, the viral vector is selected from the group consisting of a lentivirus, a retrovirus, an adenovirus, an adeno-associated virus, a cocal virus, and a baculovirus.

In some embodiments of the method, the transduction agent comprises a virus-like particle, a cell-mimicking particle, a transposon, an exosome, a nanoparticle, a micelle, and a liposome. In some embodiments of the method, the transduction agent comprises a nucleic acid cargo. In some embodiments of the method, the nucleic acid cargo comprises siRNA, tasiRNA, lncRNA, shRNA, mRNA, gRNA, miRNA, and/or viral RNA. In some embodiments of the method, the nucleic acid cargo comprises DNA that encodes a fusion protein, a chimeric antigen receptor (CAR), a therapeutic peptide or polypeptide, or a combination thereof.

Other aspects and embodiments of the disclosure will be apparent in light of the following detailed description and accompanying figures.

### BRIEF DESCRIPTION OF THE DRAWINGS

FIGS. 1A-1G: Drydux scaffolds demonstrate well-connected macroporous structure. A) Schematics showing synthesis of Drydux scaffolds. B) X-ray CT scans showing vertical (blue) and horizontal (red) cross-sectional plane of Drydux scaffold. Cross sections showing scaffold architecture with brighter values for higher density scaffold structure and darker values for lower density scaffold structure (porosity). C) Relative frequency of macropores of different diameters. D) Aspect ratio of pores (<1) suggesting predominately oblong shaped macropores. E) X-ray CT scans of Drydux scaffolds showing pore volume as highlighted space with 82.78% calculated porosity. F) X-ray CT scans showing well connected pores. Connectivity map shows individual connections of each pore shown with spheres and connecting lines. Size and color of sphere indicates number of connections. G) Quantification of pore connections showing frequency of pore connectivity.

FIGS. 2A-2C: Drydux scaffolds enable efficient static T cell reprogramming. A) Schematic showing experimental protocol to generate CAR T cells in vitro using Drydux scaffolds. B) GFP expression in primary human T cells followed by conventional or Drydux mediated reprogramming (two tailed unpaired Student's t-test) C) Stability of Drydux scaffolds was measured by storing scaffolds sealed at 4° C. and measured transduction efficiency as compared to freshly made scaffolds. (two-tailed unpaired Student's t-test). Data represents mean±SEM of three independent experiments.

FIGS. 3A-3E: Implantable Drydux scaffolds provide enhanced efficacy against lymphoma. A) Schematics showing experimental timeline of generating lymphoma xenograft model followed by treatment using implantable scaffolds or CAR T cells generated conventionally (N=5/group). Non-transduced cells seeded scaffolds were implanted as negative control (N=3). B) Tumor bioluminescent (BLI) images of lymphoma bearing NSG mice treated with non-transduced T cells or conventionally produced CAR T cells or implantation of cell and virus seeded Drydux scaffolds. C) Kinetics of tumor growth quantified by measurement of BLI signal. Thick line represents mean. D) Percent body weight changes of treated mice. Data represents mean±SD from five biologically independent samples (n=3 for non-transduced). E) Survival of treated mice (log-rank (Mantel-Cox) test and Gehan-Breslow-Wilcoxon test).

FIGS. 4A-4J: Drydux generates highly functional CAR T cells against solid tumors. A) B7H3.CAR T cells generated using conventional method and Drydux scaffold show comparable transduction efficiencies (two tailed unpaired Student's t-test). Control groups were not exposed to B7H3.CAR-encoding retrovirus. B) Representative flow plots showing B7H3.CAR expression. C) Immunophenotypic composition of B7H3.CAR T cells generated using either method at day 14 of in vitro culture (unpaired Student's t-test). D) Immunophenotypic composition of CD8 CAR T cells (unpaired Student's t-test). E) Immunophenotypic composition of CD4 CAR T cells (unpaired Student's t-test). F) Analysis of expression of exhaustion markers on B7H3.CAR T cells (unpaired Student's t-test). G) In vitro expansion of non-transduced T cells or B7H3.CAR T cells transduced using conventional methods or Drydux scaffolds. H) Percentage of residual tumor cells following co-culture of different GFP-expressing tumor cells and B7H3.CAR T cells generated using either method or non-transduced cells at 1:5 E:T ratio (one-way ANOVA with Tukey's correction). I) Quantification of IL-2 and J) IFN-γ release by CAR T cells generated using either method after 24 hours of co-culture with various tumor cells assessed by ELISA (unpaired Student's t-test). Data represents mean±SEM from three independent samples.

FIGS. 5A-5B: Implantable Drydux+IL2 scaffold provides sustained cell release in vitro. A) Schematic showing experimental setup for release study. B) Percent of initially seeded cells released from Drydux+IL2 scaffolds over 21 days (unpaired Student's t-test). Data represents mean±SEM from three independent samples.

FIGS. 6A-6B: Assessment of non-specific transduction and virus leakage outside of the Drydux scaffolds. A) Schematics showing experimental setup to assess the undesired off-scaffold transduction around an implantation site. B) GFP expression in fibroblast following potential retrovirus leakage from Drydux scaffold (two tailed unpaired Student's t-test). Data represents mean±SEM from three independent samples.

FIGS. 7A-7G: Implantable Drydux scaffolds outperform conventionally generated CAR T cells against metastatic lung tumors. A) Schematics showing experimental timeline of generating metastatic NSC lung tumor model followed by treatment using non-transduced T cells or implantable Drydux scaffold or CAR T cells generated conventionally. Donor matched T cells used for all the groups. B) BLI images of treated tumor-bearing NSG mice. C) Kinetics of tumor growth quantified by measurement of BLI signal. Thick line represents mean. D) Percent body weight change of mice during treatment. Data represents mean±SD of five biologically independent samples. E) Survival of treated mice (log-rank (Mantel-Cox) test and Gehan-Breslow-Wilcoxon test). Survival encompasses all deaths independent of tumor status. F) Number of circulating B7H3.CAR T cells analyzed on days 34 and 99 after treatment (one tailed unpaired Student's t-test). G) Immunophenotypic analysis of circulating CAR T cells on day 34 after treatment (unpaired Student's t-test). Data represents mean±SEM of four biologically independent samples.

FIGS. 8A-8E: Implantable Drydux scaffolds generate highly functional CAR T cells against ovarian tumor. A) Schematics showing experimental timeline of generating intraperitoneal ovarian xenograft model followed by treatment using donor matched CAR T cells generated using implantable Drydux scaffold in vivo or CAR T cells generated conventionally. B) BLI images of ovarian tumor bearing NSG mice treated with CAR T cells. C) Kinetics of tumor growth quantified by measurement of BLI signal. D) Body weight changes of mice during entire duration of the treatment. Data represents mean±SD of five biologically independent samples. E) Survival of treated mice (log-rank (Mantel-Cox) test and Gehan-Breslow-Wilcoxon test).

FIGS. 9A-9L: Implantable Drydux scaffolds generate highly functional and persistent CAR T cells and prevent tumor relapse in orthotopic pancreatic tumors. A) Schematic showing experimental timeline of generating orthotopic pancreatic tumor model followed by treatment using donor matched CAR T cells generated using implantable Drydux scaffolds (N=5) or CAR T cells generated conventionally (N=6). Drydux scaffolds seeded with activated PBMCs were used as non-transduced control (N=6). B) BLI images of tumor bearing NSG mice treated with non-transduced cells or conventionally produced CAR T cells or implantation of cells and virus seeded Drydux scaffolds. C) Kinetics of tumor growth quantified by measurement of BLI signal. Thick line represents mean. D) Precent body weight changes of mice during the treatment. Data represents mean±SD of five biologically independent samples. E) Survival of treated mice (log-rank (Mantel-Cox) test and Gehan-Breslow-Wilcoxon test). F) Number of circulating B7H3.CAR T cells analyzed on day 20, 40 and 123 post treatment (two tailed unpaired Student's t-test). G) immunophenotypic analysis of circulating CAR T cells on day 40 (unpaired Student's t-test) and H) day 123 post treatment. Only surviving animals treated with Drydux+IL2 scaffolds survived to be analyzed on day 123. I) Number and J) Immunophenotype of B7H3.CAR T cells in bone marrow were assessed on day 123. K) Number and L) Immunophenotype of B7H3.CAR T cells in spleen were assessed on day 123. Data represents mean±SEM of five biologically independent samples.

FIGS. 10A-10C: Characterization of Drydux scaffolds. A) X-ray CT scan showing volume of Drydux scaffold B) Pore volumes color coded to indicate pores with similar volumes. C) Surface area as function of volume plotted.

FIGS. 11A-11B: Drydux supports T cell proliferation and release. A) Proliferation of CFSE labelled cells within macroporous scaffold three days after cell seeding. B) Percent release of T cells from the macroporous scaffolds cultured in vitro in presence of exogenous cytokines. Data represents mean±SEM of three independent samples.

FIG. 12: Drydux mediates stable T cell reprogramming. B7H3.CAR expression in the conventionally or scaffold generated T cells during 14 days of in vitro culture (unpaired Student's t-test). Data represents mean±SEM of three independent samples.

FIGS. 13A-13H: Drydux scaffold produces highly functional CAR T cells in vitro. A) Expression of B7-H3 antigen on three different solid tumor cells lines (SKOV3-ovarian, A549-lung, Panc-1-pancreatic) assessed by flow cytometry. B) Representative flow graphs of co-culture of GFP-expressing Panc-1 cells with non-transduced, conventionally generated and Drydux generated B7H3.CAR T cells. C) Percentage of residual tumor cells following co-culture of GFP expressing Panc-1 and CAR T cells generated using conventional or Drydux method or non-transduced cells at 1:1 E:T ratio (one-way ANOVA with Tukey correction). D, E) Quantification of IL-2 and IFN-γ release by CAR T cells generated using either method 24 hours of co-culture with Panc-1 tumor cells at 1:1 E:T ratio assessed by ELISA (two tailed unpaired Student's t-test). F) Percentage of residual tumor cells following co-culture of different GFP expressing tumor cells and B7H3.CAR T cells generated using either method or non-transduced cells at 1:10 E:T ratio (one-way ANOVA with Tukey's correction). G, H) Quantification of IL-2 and IFN-γ release by CAR T cells generated using either method after 24 hours of co-culture with indicated tumor cells assessed by ELISA (unpaired Student's t-test). Data represents mean #SEM of three independent samples.

FIGS. 14A-14B: Drydux+IL2 promotes T cell proliferation in vitro. A) GFP expression in pre-activated T cells two days post transduction seeded on Drydux and Drydux+IL2 (two tailed unpaired Student's t-test) B) Cell proliferation assessed on day of seeding (day 0) and 2 days following seeding on Drydux and Drydux+IL2 (unpaired Student's t-test). Data represents mean±SEM of three independent samples.

FIGS. 15A-15G: Drydux generated CAR T cells show superior persistence in vivo in metastatic lung tumor model. A) Schematics showing details of experimentation. Donor matched T cells were used to generate CAR T cells using implantable Drydux scaffolds or conventional method involving RetroNectin and spinoculation followed by in vitro expansion. FFluc expressing tumor cells were inoculated 14 days before initiation of the respective treatment B) Representative flow graphs showing gating strategy to determine CAR T cells number and their phenotype in blood and lymphoid organs. C) Phenotype of CAR T cells in blood on day 99 post treatment D) Number of CAR T cells present in spleen (two tailed unpaired Student's t-test) and E) Bone marrow assessed on day 99 post treatment (two tailed unpaired Student's t-test). F) Phenotype of CAR T cells in spleen (unpaired Student's t-test) and G) Bone marrow on day 99 post treatment (unpaired Student's t-test). Data represents mean±SEM of two biologically independent samples.

FIGS. 16A-16F: Drydux generated CAR T cells show improved persistence in vivo in an intraperitoneal ovarian tumor model. A) Schematics showing details of experimentation. T cells from the same donor were used to generate CAR T cells using implantable Drydux scaffolds or conventional method involving RetroNectin coating, spinoculation and in vitro expansion. Scaffolds seeded with only cells were used as negative control. FFluc expressing tumor cells were inoculated 14 days before initiation of the respective treatment. tumor growth was monitored weekly using IVIS imaging. B) Number of circulating B7H3.CAR T cells in blood at day 126 post treatment. C) Number of B7H3.CAR T cells in bone marrow were assessed on day 126. D) Number of B7H3.CAR T cells in spleen were assessed on day 126. Data represents mean±SEM of biologically independent samples. E) Number of circulating B7H3.CAR T cells assessed at day 34 post treatment (two tailed unpaired Student's t-test). F) Immunophenotypic analysis of circulating B7H3.CAR T cells (unpaired Student's t-test).

FIGS. 17A-17B: Drydux generated CAR T cells improved tumor free survival preventing relapse in orthotopic pancreatic tumor model. A) Schematics showing details of experimentation. Donor matched T cells were used to generate CAR T cells using implantable Drydux scaffolds or conventional method involving RetroNectin and spinoculation followed by in vitro expansion. FFluc expressing tumor cells were inoculated 12 days before initiation of the respective treatment and tumor growth was monitored weekly. B) Tumor free survival of animals receiving conventionally generated CAR T cells or Drydux mediated in vivo generated CAR T cells (log-rank (Mantel-Cox) test and Gehan-Breslow-Wilcoxon test).

FIG. 18: Fabrication of dry macroporous alginate (Drydux) scaffolds. An alginate solution is cross-linked with a calcium solution and the resulting gel is frozen overnight followed by lyophilization for 72 h to create dry macroporous scaffolds. Activated T cells and viral particles are mixed and seeded on top of the scaffold and scaffolds are incubated at 37° C., 5% CO₂. EDTA is used to dissolve the scaffolds and isolate the transduced T cells.

FIGS. 19A-19F: Impact of porosity and stiffness on Drydux transduction efficiency varying calcium and alginate concentrations. (A) Photographs of scaffolds with corresponding SEM images and average pore sizes. (B) Quantification of retrovirus transduction efficiency against primary human PBMCs for each calcium-alginate combination with significance shown between differing calcium concentrations; * p<0.0001 with all other p-values indicated on plot; concentrations used were ˜5000 cells/μL and ˜10000 viruses/μL; n=3 scaffolds per group; two-way ANOVA with Tukey correction used to determine significance. See FIGS. 25A-25E for significance between differing alginate concentrations. (C) Quantification of scaffold pore size using a minimum of 10 pores per scaffold. (D) Spearman correlation between scaffold pore size and transduction efficiency. (E) Quantification of Young's modulus of each scaffold; n=3 scaffolds per group. (F) Spearman correlation between scaffold stiffness and transduction efficiency. Data are represented as the mean±SEM. Statistical analysis was not completed for (C) or (E).

FIGS. 20A-20F: Impact of porosity and stiffness on Drydux transduction efficiency varying freezing temperature and alginate concentration. (A) Photographs of scaffolds with corresponding SEM images and average pore sizes. (B) Quantification of retroviral transduction efficiency against primary human PBMCs for each alginate-temperature combination with significance shown between differing temperatures; * p<0.0001 with all other p-values indicated on plot; concentrations used were ˜5000 cells/μL and ˜10000 viruses/μL; n=3 scaffolds per group; two-way ANOVA with Tukey correction used to determine significance. See FIGS. 26A-26D for significance between differing alginate concentrations. (C) Quantification of scaffold pore size using a minimum of 10 pores per scaffold. (D) Spearman correlation between scaffold pore size and transduction efficiency. (E) Quantification of Young's modulus of each scaffold; n=3 scaffolds per group. (F) Spearman correlation between scaffold stiffness and transduction efficiency. Data are represented as the mean±SEM. Statistical analysis was not completed for (C) or (E).

FIGS. 21A-21G: Impact of seed volume on Drydux transduction. (A) Live-images of scaffold absorbing 20 μL of cell-virus solution. (B) Images of scaffolds 24 hours after absorbing different volumes of cell-virus solution. (C) Quantification of transduction efficiency for each seed volume. (D) Kinetics of absorption for each seed volume. (E) Spearman correlation between absorption rate and transduction efficiency. (F) Calculated volumetric flux of different seed volumes. (G) Spearman correlation between volumetric flux and transduction efficiency. Data are represented as the mean±SEM; concentrations used were ˜2000 cells/μL and ˜4000 viruses/μL; n=3 scaffolds per group; one-way ANOVA was used to determine significance.

FIGS. 22A-22D: Computational model of flow through scaffold pore. (A) Schematic showing activated T cells and virus seeded together onto dry macroporous scaffold. (B) Particle positions at a statistical equilibrium state for uniform unbounded flow (top) and flow inside the scaffold pore at a volumetric flux of 30 μL/min/cm² (bottom). (C) The flow velocity distribution at the midplane of the scaffold model showing the flow acceleration and deceleration in response to the changes in the model geometry. (D) Quantification of the number of collisions per 1 μL per minute for no flow, unbounded flow, and scaffold pore flow at different volumetric fluxes.

FIGS. 23A-23B: Representative flow cytometry results and gating strategy for (A) non-transduced cells and (B) GFP+ cells.

FIG. 24: Quantification of transduction efficiency for different MOI (ratio of the number of viral particles to activated T cells) values. Data are represented as the mean±SEM; concentrations used were ˜5000 cells/μL; n=2 scaffolds per group.

FIGS. 25A-25E: Further characterization of biomaterial scaffolds synthesized with varying calcium and alginate concentrations. (A-C) Stress-strain curves generated from compression testing of different calcium-alginate scaffolds. (D) Quantification of transduction efficiency for each calcium-alginate combination with significance shown between differing alginate concentrations; data are represented as the mean±SEM; concentrations used were ˜5000 cells/μL and ˜10000 viruses/μL; n=3 scaffolds per group; two-way ANOVA with Tukey correction used to determine significance.

FIGS. 26A-26D: Further characterization of biomaterial scaffolds synthesized with varying alginate concentrations and freezing temperatures. (A-C) Stress-strain curves generated from compression testing of different alginate-temperature scaffolds. (D) Quantification of transduction efficiency for each alginate-temperature combination with significance shown between differing alginate concentrations; data are represented as the mean±SEM; concentrations used were ˜5000 cells/μL and ˜10000 viruses/μL; n=3 scaffolds per group; two-way ANOVA with Tukey correction used to determine significance.

FIG. 27: Quantification of cell and viral particle concentration on transduction efficiency. Data are represented as the mean±SEM; n=4 scaffolds per group; one-way ANOVA with Tukey correction used to determine significance.

FIGS. 28A-28C: Preliminary experiment testing seed volume and corresponding absorption rate on transduction efficiency. (A) Kinetics of absorption of different seed volumes. (B) Quantification of transduction efficiency for each seed volume. (C) Spearman correlation between absorption rate of cell-virus solution and transduction efficiency. Data are represented as the mean±SEM; concentrations used were ˜2000 cells/μL and ˜4000 viruses/μL; n=4 scaffolds per group; one-way ANOVA with Tukey correction used to determine significance.

FIGS. 29A-29B: Impact of surface area on transduction efficiency. (A) Scaffolds were created in 6-well plates and seeded with primary human T cells and concentrated GFP retrovirus at an MOI of 4. The cell-virus solution was either spread out on the entire surface of the scaffold or seeded in a single location on the scaffold. (B) Quantification of transduction efficiency for each group. Data are represented as mean±SEM; n=3 per group; unpaired t-test with Welch's correction used to determine significance.

### DETAILED DESCRIPTION

Embodiments of the present disclosure provide compositions, systems, and methods related to cellular transduction. In particular, the present disclosure provides compositions, systems, and methods pertaining to implantable macroporous scaffolds that facilitate rapid and highly efficient cellular transduction. In accordance with these embodiments, the present discourse demonstrates that implantable macroporous scaffolds that can be tuned to incorporate cell proliferation and release cues to efficiently reprogram T cells and release CAR T cells for the treatment of solid tumors. The compositions and systems described herein require minimal ex vivo manipulation and are designed to be implantable within three days of T cell isolation, providing sufficient time for the clinically required preconditioning and lymphodepletion of patient T cells. As described further herein, the implantable macroporous scaffolds of the present disclosure mediate in vitro and in vivo T cell reprogramming and promote CAR T cell proliferation and release. These scaffolds were highly efficacious in animal models of systemic lymphoma, intravascular metastatic lung cancer, intraperitoneal metastatic ovarian cancer, and in an orthotopic pancreatic cancer. These solid tumors have shown poor prognosis and limited improvement in 5-year survival rates despite the advancements in various therapeutic options. The implantable macroporous scaffolds drastically decreased the time and effort needed to generate CAR T cells. In addition, the implantable macroporous scaffolds improved CAR T cell persistence, providing enhanced efficacy compared to equal numbers of conventionally generated CAR T cells.

Additionally, the results and data provided herein demonstrate that dry macroporous alginate (“Drydux”) scaffolds can improve viral T cell transduction and other hard-to-transduce cells. As described further herein, experiments were conducted to elucidate the mechanism behind Drydux scaffold function through a study of the impact of pore size, scaffold stiffness, virus concentration, and absorption volume on transduction efficiency. Results demonstrated that scaffold pore size has a complicated effect on transduction efficiency, and further demonstrated that scaffold stiffness does not impact Drydux transduction. It was discovered that more concentrated virus suspensions led to higher transduction efficiencies, suggesting that in the future cell-virus solution should be as concentrated as possible for optimal transduction efficiency. Interestingly, a strong correlation was found between absorption speed and transduction efficiency. Finally, experimental results were validated using a computational model of cell and virus collisions flowing through a porous scaffold. Based on these results, absorption speed and volumetric flux appear to be important components of Drydux transduction.

Furthermore, results indicated that scaffold stiffness does not appear to contribute significantly to Drydux transduction. Scaffold stiffness has been shown to effect migration and differentiation of cells within the scaffolds, influencing cell infiltration into host tissues. Studies have demonstrated that softer matrices induce higher T cell proliferation and mechanotransduction required for T cell receptor signaling. This is because softer scaffolds usually have higher porosity and interconnectivity, which promotes more interaction among the cells, leading to higher T cell transduction. However, in these examples, scaffold stiffness was altered using adherent cells through cell-adhesion peptides, such as RGD. In the case of Drydux scaffolds, the unmodified alginate does not present adhesion ligands to the T cells, which are themselves non-adhesive cells, likely explaining the lack of impact of scaffold stiffness on Drydux transduction.

The impact of pore size on transduction was less straightforward. SEM imaging was used to determine pore sizes due to the accessibility and high throughput that SEM imaging provides. However, SEM only provides surface porosity and future studies could focus on more rigorous pore quantification methods, including microCT, Brunauer-Emmett-Teller (BET) surface area analysis, or porosimetry. Smaller pores would lead to a larger transduction efficiency, as scaffolds with smaller pores usually have a larger porosity and more interconnectivity that allows for greater cell and virus interaction and enhanced diffusion of nutrients and oxygen. Although a significant correlation between smaller pore size and transduction was observed when changing the calcium concentration, this correlation disappeared when the pore size was controlled by freezing temperature. Therefore, pore size may still play an important role in cell transduction.

Overall, Drydux transduction is a robust process that remained efficient across a wide array of alginate concentrations, calcium concentrations, and freezing temperatures. The robust nature of the system gives more credence to the possibility that Drydux scaffolds can find utility in the production of cellular therapies and specifically benefit CAR T cell therapies for solid tumors, where tuning scaffold mechanics to the mechanics of the implanted tissue could improve the success of treatment. This becomes highly important when treating specific solid tumors, such as glioblastoma, where matching the scaffold stiffness to that of the brain can affect cell viability, migration, and infiltration into surrounding tissues.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transduced,” “transformed,” or “transfected” by exogenous DNA, e.g., a recombinant expression vector, when such DNA has been introduced inside the cell. The presence of exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Reduce” or other forms of the word, such as “reducing” or “reduction,” generally means a lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician (e.g., physician).

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, “alginate” generally refers to a salt or ester of alginic acid. Alginate is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks), alternating M and G-residues (MG-blocks) or randomly organized blocks. The relative amount of each block type varies both with the origin of the alginate and the concentration of G and M acids (the “G/M ratio”), and thus contributes to varied structural and biocompatibility characteristics. For example, alternating blocks form the most flexible chains, and are more soluble at lower pH than the other blocks. G-blocks form stiff chain elements, and two G-blocks of more than 6 residues each can form stable cross-linked junctions with divalent cations (e.g., Ca²⁺, Mg²⁺, Ba²⁺, Sr²⁺ among others), leading to a three-dimensional gel network.

As used herein, “Young's modulus” or “Young's elastic modulus” is the mechanical property that measures the stiffness of a material and can be expressed in, for example, kilopascals of pressure (kPa). As described further herein, the stiffness of particular tissues vary throughout the human body, and this property can elicit various biochemical and cellular responses. Embodiments of the present disclosure include the generation of implantable macroporous scaffolds comprised of a biopolymer matrix that can be tuned to match or be compatible with the stiffness of any target tissue.

Certain methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. COMPOSITIONS

Embodiments of the present disclosure include compositions, systems, and methods related to cellular transduction. In particular, the present disclosure provides compositions, systems, and methods pertaining to implantable macroporous scaffolds that facilitate rapid and highly efficient cellular transduction. In accordance with these embodiments, the present disclosure includes an implantable macroporous scaffold comprising a crosslinked biopolymer matrix and a composition comprising a plurality of cells and a transduction agent. In some embodiments, the crosslinked biopolymer matrix comprises an average pore size ranging from about 10 μm to about 500 μm, and a stiffness ranging from about 1 kPa to about 1000 kPa. In some embodiments, the stiffness of the matrix is compatible with the stiffness of a target tissue, and the scaffold facilitates transduction of the plurality of cells with the transduction agent.

As described further herein, the implantable macroporous scaffolds of the present disclosure are comprised of a crosslinked biopolymer matrix, which is designed such that the stiffness of the crosslinked biopolymer matrix is compatible with that of a target tissue. As would be appreciated by one of ordinary skill in the art based on the present disclosure, the stiffness of a particular target tissue (e.g., as measured using Young's modulus) is one important factor to consider when generating and implanting biomaterials into a subject. For example, in some embodiments, the target tissue is nervous tissue (e.g., brain, spinal cord, sciatic nerve, ulnar nerve, and the like) having a stiffness ranging from about 0.4 kPa to about 7 kPa, and the scaffolds of the present disclosure can be constructed to have a compatible stiffness without compromising transduction efficiency. In some embodiments, the target tissue is connective tissue (e.g., tibial bone, femoral bone, articular cartilage, adipose tissue, patellar tendon, ligaments, and the like) having a stiffness ranging from about 2 kPa to about 21 GPa, and the scaffolds of the present disclosure can be constructed to have a compatible stiffness without compromising transduction efficiency. In some embodiments, the target tissue is muscle tissue (e.g., smooth muscle, cardiac muscle, skeletal muscle, and the like) having a stiffness ranging from about 2 kPa to about 800 kPa, and the scaffolds of the present disclosure can be constructed to have a compatible stiffness without compromising transduction efficiency. In some embodiments, the target tissue is endothelial and epithelial tissue (e.g., skin, lung, intestines, and the like) having a stiffness ranging from about 1 kPa to about 14 MPa, and the scaffolds of the present disclosure can be constructed to have a compatible stiffness without compromising transduction efficiency. In some embodiments, the target tissue is viscera (e.g., kidney, spleen, liver, thymus, thyroid, pancreas, bladder, and the like) having a stiffness ranging from about 0.1 kPa to about 300 kPa, and the scaffolds of the present disclosure can be constructed to have a compatible stiffness without compromising transduction efficiency. In some embodiments, the target tissue is eye tissue (e.g., cornea, lens, and the like) having a stiffness ranging from about 4 kPa to about 4 MPa, and the scaffolds of the present disclosure can be constructed to have a compatible stiffness without compromising transduction efficiency. As would be understood by one of ordinary skill in the art, the scaffolds of the present disclosure can be constructed to have a stiffness that is compatible with any target tissue without compromising transduction efficiency. In some embodiments, the target tissue is or comprises tissue that exhibits characteristics that are consistent with a disease or condition, including but not limited to, cancerous tissue (e.g., solid tumor tissue).

In accordance with these embodiments, the stiffness of the crosslinked biopolymer matrix of the present disclosure can range from about 1 kPa to about 1000 kPa. In some embodiments, the stiffness of the crosslinked biopolymer matrix is from about 1 kPa to about 900 kPa. In some embodiments, the stiffness of the crosslinked biopolymer matrix is from about 1 kPa to about 800 kPa. In some embodiments, the stiffness of the crosslinked biopolymer matrix is from about 1 k Pa to about 700 kPa. In some embodiments, the stiffness of the crosslinked biopolymer matrix is from about 1 kPa to about 600 kPa. In some embodiments, the stiffness of the crosslinked biopolymer matrix is from about 1 kPa to about 500 kPa. In some embodiments, the stiffness of the crosslinked biopolymer matrix is from about 1 kPa to about 400 kPa. In some embodiments, the stiffness of the crosslinked biopolymer matrix is from about 1 kPa to about 300 kPa. In some embodiments, the stiffness of the crosslinked biopolymer matrix is from about 1 kPa to about 200 kPa. In some embodiments, the stiffness of the crosslinked biopolymer matrix is from about 1 kPa to about 100 kPa. In some embodiments, the stiffness of the crosslinked biopolymer matrix is from about 1 kPa to about 50 kPa. In some embodiments, the stiffness of the crosslinked biopolymer matrix is from about 10 kPa to about 1000 kPa. In some embodiments, the stiffness of the crosslinked biopolymer matrix is from about 50 kPa to about 1000 kPa. In some embodiments, the stiffness of the crosslinked biopolymer matrix is from about 100 kPa to about 1000 kPa. In some embodiments, the stiffness of the crosslinked biopolymer matrix is from about 200 kPa to about 1000 kPa. In some embodiments, the stiffness of the crosslinked biopolymer matrix is from about 300 kPa to about 1000 kPa. In some embodiments, the stiffness of the crosslinked biopolymer matrix is from about 400 kPa to about 1000 kPa. In some embodiments, the stiffness of the crosslinked biopolymer matrix is from about 500 kPa to about 1000 kPa. In some embodiments, the stiffness of the crosslinked biopolymer matrix is from about 600 kPa to about 1000 kPa. In some embodiments, the stiffness of the crosslinked biopolymer matrix is from about 700 kPa to about 1000 kPa. In some embodiments, the stiffness of the crosslinked biopolymer matrix is from about 800 kPa to about 1000 kPa. In some embodiments, the stiffness of the crosslinked biopolymer matrix is from about 900 kPa to about 1000 kPa. In some embodiments, the stiffness of the crosslinked biopolymer matrix is from about 50 kPa to about 500 kPa. In some embodiments, the stiffness of the crosslinked biopolymer matrix is from about 100 kPa to about 500 kPa. In some embodiments, the stiffness of the crosslinked biopolymer matrix is from about 250 kPa to about 750 kPa. In some embodiments, the stiffness of the crosslinked biopolymer matrix is from about 300 kPa to about 600 kPa. In some embodiments, the stiffness of the crosslinked biopolymer matrix is from about 500 kPa to about 800 kPa. In some embodiments, the stiffness of the crosslinked biopolymer matrix is from about 600 kPa to about 900 kPa. In some embodiments, the stiffness of the crosslinked biopolymer matrix is from about 25 kPa to about 650 kPa. In some embodiments, the stiffness of the crosslinked biopolymer matrix is from about 0.2 kPa to about 65 kPa.

In other embodiments, the stiffness of the crosslinked biopolymer matrix is from about 0.1 kPa to about 10 kPa. In some embodiments, the stiffness of the crosslinked biopolymer matrix is from about 0.1 kPa to about 9 kPa. In some embodiments, the stiffness of the crosslinked biopolymer matrix is from about 0.1 kPa to about 8 kPa. In some embodiments, the stiffness of the crosslinked biopolymer matrix is from about 0.1 kPa to about 7 kPa. In some embodiments, the stiffness of the crosslinked biopolymer matrix is from about 0.1 kPa to about 6 kPa. In some embodiments, the stiffness of the crosslinked biopolymer matrix is from about 0.1 kPa to about 5 kPa. In some embodiments, the stiffness of the crosslinked biopolymer matrix is from about 0.1 kPa to about 4 kPa. In some embodiments, the stiffness of the crosslinked biopolymer matrix is from about 0.1 kPa to about 3 kPa. In some embodiments, the stiffness of the crosslinked biopolymer matrix is from about 0.1 kPa to about 2 kPa. In some embodiments, the stiffness of the crosslinked biopolymer matrix is from about 0.1 kPa to about 1 kPa. In some embodiments, the stiffness of the crosslinked biopolymer matrix is from about 0.1 kPa to about 0.5 kPa. In some embodiments, the stiffness of the crosslinked biopolymer matrix is from about 0.5 kPa to about 9 kPa. In some embodiments, the stiffness of the crosslinked biopolymer matrix is from about 1 kPa to about 9 kPa. In some embodiments, the stiffness of the crosslinked biopolymer matrix is from about 2 kPa to about 9 kPa. In some embodiments, the stiffness of the crosslinked biopolymer matrix is from about 3 kPa to about 9 kPa. In some embodiments, the stiffness of the crosslinked biopolymer matrix is from about 4 kPa to about 9 kPa. In some embodiments, the stiffness of the crosslinked biopolymer matrix is from about 5 kPa to about 9 kPa. In some embodiments, the stiffness of the crosslinked biopolymer matrix is from about 6 kPa to about 9 kPa. In some embodiments, the stiffness of the crosslinked biopolymer matrix is from about 7 kPa to about 9 kPa. In some embodiments, the stiffness of the crosslinked biopolymer matrix is from about 8 kPa to about 9 kPa. In some embodiments, the stiffness of the crosslinked biopolymer matrix is from about 1 kPa to about 8 kPa. In some embodiments, the stiffness of the crosslinked biopolymer matrix is from about 2 kPa to about 6 kPa. In some embodiments, the stiffness of the crosslinked biopolymer matrix is from about 3 kPa to about 5 kPa. In some embodiments, the stiffness of the crosslinked biopolymer matrix is from about 2 kPa to about 4 kPa. In some embodiments, the stiffness of the crosslinked biopolymer matrix is from about 4 kPa to about 6 kPa. In some embodiments, the stiffness of the crosslinked biopolymer matrix is from about 5 kPa to about 7 kPa.

The stiffness or elasticity (e.g., Young's modulus) of varying tissues has been described previously. Thus, in some embodiments, the implantable macroporous scaffolds of the present disclosure can exhibit a Young's modulus compatible with the tissue in which it is implanted. This can improve compatibility between the implantable scaffold and the host target tissue, and the scaffolds will still exhibit acceptable transduction efficiencies. For example, the macroporous scaffolds of the present disclosure, when hydrated with a composition comprising a plurality of cells and a transduction agent, can exhibit a stiffness that is from about ±25%, about ±50%, about ±75%, about ±100%, about ±125%, about ±150%, about ±175%, about ±200%, about ±225%, or about ±250% of the stiffness of the target tissue.

In accordance with these embodiments, the scaffold is implanted within or adjacent to the target tissue. In some embodiments, the target tissue is tumor tissue. In some embodiments, the target tissue is solid tumor tissue. In some embodiments, the target tissue comprises at least one of lung tissue, bone tissue, skin tissue, breast tissue, muscle tissue, nerve tissue, brain tissue, lymph tissue, prostate tissue, bladder tissue, stomach tissue, intestinal tissue, uterine tissue, ovarian tissue, liver tissue, adipose tissue, cartilaginous tissue, thyroid tissue, and/or pancreatic tissue.

In some embodiments, the scaffold facilitates the transduction of a plurality of cells (e.g., immune cells) with a transduction agent (e.g., viral vector containing a polynucleotide encoding a protein-of-interest) with a transduction efficiency of at least 50% (measured in vivo or ex vivo). In some embodiments, the scaffold facilitates the transduction of the plurality of cells with the transduction agent with a transduction efficiency of at least 60%. In some embodiments, the scaffold facilitates the transduction of the plurality of cells with the transduction agent with a transduction efficiency of at least 70%. In some embodiments, the scaffold facilitates the transduction of the plurality of cells with the transduction agent with a transduction efficiency of at least 80%. In some embodiments, the scaffold facilitates the transduction of the plurality of cells with the transduction agent with a transduction efficiency of at least 90%. In some embodiments, the scaffold facilitates the transduction of the plurality of cells with the transduction agent with a transduction efficiency of about 50% to about 90%. In some embodiments, the scaffold facilitates the transduction of the plurality of cells with the transduction agent with a transduction efficiency of about 60% to about 90%. In some embodiments, the scaffold facilitates the transduction of the plurality of cells with the transduction agent with a transduction efficiency of about 70% to about 90%. In some embodiments, the scaffold facilitates the transduction of the plurality of cells with the transduction agent with a transduction efficiency of about 50% to about 80%. In some embodiments, the scaffold facilitates the transduction of the plurality of cells with the transduction agent with a transduction efficiency of about 50% to about 70%. In some embodiments, the scaffold facilitates the transduction of the plurality of cells with the transduction agent with a transduction efficiency of about 60% to about 80%. In some embodiments, the scaffold facilitates the transduction of the plurality of cells with the transduction agent with a transduction efficiency of about 70% to about 90%.

In addition to stiffness, and as described further herein, average pore size of the crosslinked biopolymer matrix of the implantable macroporous scaffolds of the present disclosure is another important factor to consider when generating and implanting biomaterials into a subject sufficient to facilitate cell transduction. In some embodiments, the crosslinked biopolymer matrix has an average pore size ranging from about 10 μm to about 500 μm. In some embodiments, the crosslinked biopolymer matrix has an average pore size ranging from about 10 μm to about 450 μm. In some embodiments, the crosslinked biopolymer matrix has an average pore size ranging from about 10 μm to about 400 μm. In some embodiments, the crosslinked biopolymer matrix has an average pore size ranging from about 10 μm to about 350 μm. In some embodiments, the crosslinked biopolymer matrix has an average pore size ranging from about 10 μm to about 300 μm. In some embodiments, the crosslinked biopolymer matrix has an average pore size ranging from about 10 μm to about 250 μm. In some embodiments, the crosslinked biopolymer matrix has an average pore size ranging from about 10 μm to about 200 μm. In some embodiments, the crosslinked biopolymer matrix has an average pore size ranging from about 10 μm to about 150 μm. In some embodiments, the crosslinked biopolymer matrix has an average pore size ranging from about 10 μm to about 100 μm. In some embodiments, the crosslinked biopolymer matrix has an average pore size ranging from about 10 μm to about 50 μm. In some embodiments, the crosslinked biopolymer matrix has an average pore size ranging from about 10 μm to about 25 μm. In some embodiments, the crosslinked biopolymer matrix has an average pore size ranging from about 25 μm to about 500 μm. In some embodiments, the crosslinked biopolymer matrix has an average pore size ranging from about 50 μm to about 500 μm. In some embodiments, the crosslinked biopolymer matrix has an average pore size ranging from about 100 μm to about 500 μm. In some embodiments, the crosslinked biopolymer matrix has an average pore size ranging from about 150 μm to about 500 μm. In some embodiments, the crosslinked biopolymer matrix has an average pore size ranging from about 200 μm to about 500 μm. In some embodiments, the crosslinked biopolymer matrix has an average pore size ranging from about 250 μm to about 500 μm. In some embodiments, the crosslinked biopolymer matrix has an average pore size ranging from about 300 μm to about 500 μm. In some embodiments, the crosslinked biopolymer matrix has an average pore size ranging from about 350 μm to about 500 μm. In some embodiments, the crosslinked biopolymer matrix has an average pore size ranging from about 400 μm to about 500 μm. In some embodiments, the crosslinked biopolymer matrix has an average pore size ranging from about 450 μm to about 500 μm. In some embodiments, the crosslinked biopolymer matrix has an average pore size ranging from about 50 μm to about 400 μm. In some embodiments, the crosslinked biopolymer matrix has an average pore size ranging from about 100 μm to about 300 μm. In some embodiments, the crosslinked biopolymer matrix has an average pore size ranging from about 200 μm to about 400 μm. In some embodiments, the crosslinked biopolymer matrix has an average pore size ranging from about 150 μm to about 350 μm. In some embodiments, the crosslinked biopolymer matrix has an average pore size ranging from about 200 μm to about 300 μm. In some embodiments, the crosslinked biopolymer matrix has an average pore size ranging from about 50 μm to about 250 μm. In some embodiments, the crosslinked biopolymer matrix has an average pore size ranging from about 100 μm to about 200 μm. In some embodiments, the crosslinked biopolymer matrix has an average pore size ranging from about 50 μm to about 150 μm.

The implantable macroporous scaffolds of the present disclosure can be constructed out of a variety of different rigid, semi-rigid, flexible, gel, self-assembling, liquid crystalline, or fluid compositions, including but not limited to, peptide polymers, polysaccharides, synthetic polymers, ceramics (e.g., calcium phosphate or hydroxyapatite), proteins, glycoproteins, proteoglycans, metals and metal alloys. The compositions can be assembled into scaffold using methods known in the art, e.g., injection molding, lyophilization of preformed structures, printing, self-assembly, phase inversion, solvent casting, melt processing, gas foaming, fiber forming/processing, particulate leaching or a combination thereof.

In some embodiments, the implantable macroporous scaffolds disclosed herein can be made using any suitable biodegradable polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

Exemplary materials used to form the implantable macroporous scaffolds of the present disclosure, include (but are not limited to) polylactic acid, polyglycolic acid, poly-lactide-co-glycolide (PLG), alginates and alginate derivatives, gelatin, collagen, fibrin, fibronectin, methacrylamide, acrylamide, decellularized tissues, hyaluronic acid, laminin rich gels, agarose, natural and synthetic polysaccharides, polyamino acids, polypeptides, polyesters, polyanhydrides, polyphosphazines, poly(vinyl alcohols), poly(alkylene oxides), poly(allylamines) (PAM), poly(acrylates), modified styrene polymers, pluronic polyols, polyoxamers, poly(uronic acids), poly(vinylpyrrolidone) and copolymers or graft copolymers of any of the above. In some embodiments, the biopolymer matrix comprises at least one of alginate, Hyaluronic acid, collagen, fibrin, Poly Lactic-co-Glycolic Acid (PLGA), Polycaprolactone (PCL), gelatin, Polyethylene glycol (PEG), chitosan, cellulose, polyglutamic acid, fibrin, silk, agarose, dextran, polyacrylamide, polyvinyl alcohol, Poly(N-isopropylacrylamide), Poly(2-hydroxyethyl methacrylate), polyurethane, polyethyleneimine, Poly(methyl methacrylate, Poly(2-oxazoline), Polyphosphazenes, and any composites, derivatives, or combinations thereof.

In some embodiments, hydrogel includes an RGD-modified alginate. Thus, also disclosed herein are macroporous scaffolds, wherein the scaffold comprises a crosslinked hydrogel and/or a crosslinked biopolymer, such as alginate. In some embodiments, the macroporous scaffold includes crosslinked polymers, e.g., crosslinked alginates, gelatins, or derivatives thereof, such as those that are methacrylated.

In some embodiments, the macroporous scaffold can comprise a biocompatible polymer (such as, for example, alginate). Such polymers can also serve to slowly release CAR T cell, CAR NK cell, TIL, and/or MIL into the tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly(gly colic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments, the particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein can comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

As indicated above, one material for the implantable macroporous scaffolds of the present disclosure is alginate or modified alginate material. Alginates are versatile polysaccharide based polymers that may be formulated for specific applications by controlling the molecular weight, rate of degradation and method of scaffold formation. Alginate molecules are comprised of (1-4)-linked β-D-mannuronic acid (M units) and α L-guluronic acid (G units) monomers, which can vary in proportion and sequential distribution along the polymer chain. Alginate polysaccharides are polyelectrolyte systems which have a strong affinity for divalent cations (e.g., Ca⁺², Mg⁺², Ba⁺²) and form stable scaffolds when exposed to these molecules. See Martinsen A., et al., Biotech. & Bioeng., 33 (1989) 79-89. For example, calcium cross-linked alginate scaffolds are useful for the methods described herein. For example, the polymers, e.g., alginates, of the hydrogel are 0-100% crosslinked, e.g., at least 1%, 5%, 10%, 20%, 30%, 40%, 50%, 60%, 70%, 80%, 90%, or more, crosslinked. In other embodiments, the polymers, e.g., alginates, of the scaffold are not crosslinked. In some examples, the polymers, e.g., alginates, of the scaffold contain less than 50%, e.g., less than 50%, 40%, 30%, 20%, 10%, 50%, 2%, 1%, or less, crosslinking.

Alginate may be chemically modified to yield new properties. For example, alginate may be oxidized to increase the rate of biodegradation. Alternatively, alginate may be reduced for improved biocompatibility. Alginate can also be chemically modified to change their crosslinking behavior. For instance, alginate can be modified with bioorthogonal click groups to allow click crosslinking. In another example, alginate can be modified with acrylic groups to allow radical polymerization crosslinking. As another example, alginates can be modified with host-guest chemistries to allow host-guest crosslinking.

Alginate polymers are formed into a variety of scaffold types. Alginate scaffolds can be formed from alginate with molecular weight varying between 1,000 Da to 500,000 Da. Alginate scaffolds can be formed from alginate containing a G/M ratio of between 0.5 and 5. Differences in hydrogel formulation control the kinetics of scaffold degradation. Release rates of pharmaceutical compositions, e.g., small molecules, morphogens, or other bioactive substances, from alginate macroporous scaffolds is controlled by the scaffold formulation to present the pharmaceutical compositions in a spatially and temporally controlled manner. This controlled release eliminates systemic side effects and the need for multiple injections. Useful polysaccharides other than alginates include but are not limited to agarose and microbial polysaccharides such as: Fungal Pullulan, Scleroglucan, Chitin, Chitosan, Elsinan, Bacterial Xanthan gum, Curdlan, Dextran, Gelatin, Levan, Emulsan, Cellulose, Hyaluronic Acid and others.

In accordance with the above embodiments, the biopolymer matrix comprises alginate having a molecular weight from about 1 kDa to about 500 kDa. In some embodiments, the biopolymer matrix comprises alginate having a molecular weight from about 1 kDa to about 450 kDa. In some embodiments, the biopolymer matrix comprises alginate having a molecular weight from about 1 kDa to about 400 kDa. In some embodiments, the biopolymer matrix comprises alginate having a molecular weight from about 1 kDa to about 350 kDa. In some embodiments, the biopolymer matrix comprises alginate having a molecular weight from about 1 kDa to about 300 kDa. In some embodiments, the biopolymer matrix comprises alginate having a molecular weight from about 1 kDa to about 350 kDa. In some embodiments, the biopolymer matrix comprises alginate having a molecular weight from about 1 kDa to about 300 kDa. In some embodiments, the biopolymer matrix comprises alginate having a molecular weight from about 1 kDa to about 250 kDa. In some embodiments, the biopolymer matrix comprises alginate having a molecular weight from about 1 kDa to about 200 kDa. In some embodiments, the biopolymer matrix comprises alginate having a molecular weight from about 1 kDa to about 150 kDa. In some embodiments, the biopolymer matrix comprises alginate having a molecular weight from about 1 kDa to about 100 kDa. In some embodiments, the biopolymer matrix comprises alginate having a molecular weight from about 1 kDa to about 50 kDa. In some embodiments, the biopolymer matrix comprises alginate having a molecular weight from about 50 kDa to about 500 kDa. In some embodiments, the biopolymer matrix comprises alginate having a molecular weight from about 100 kDa to about 500 kDa. In some embodiments, the biopolymer matrix comprises alginate having a molecular weight from about 150 kDa to about 500 kDa. In some embodiments, the biopolymer matrix comprises alginate having a molecular weight from about 200 kDa to about 500 kDa. In some embodiments, the biopolymer matrix comprises alginate having a molecular weight from about 250 kDa to about 500 kDa. In some embodiments, the biopolymer matrix comprises alginate having a molecular weight from about 300 kDa to about 500 kDa. In some embodiments, the biopolymer matrix comprises alginate having a molecular weight from about 350 kDa to about 500 kDa. In some embodiments, the biopolymer matrix comprises alginate having a molecular weight from about 400 kDa to about 500 kDa. In some embodiments, the biopolymer matrix comprises alginate having a molecular weight from about 450 kDa to about 500 kDa. In some embodiments, the biopolymer matrix comprises alginate having a molecular weight from about 100 kDa to about 400 kDa. In some embodiments, the biopolymer matrix comprises alginate having a molecular weight from about 200 kDa to about 400 kDa. In some embodiments, the biopolymer matrix comprises alginate having a molecular weight from about 100 kDa to about 300 kDa. In some embodiments, the biopolymer matrix comprises alginate having a molecular weight from about 150 kDa to about 350 kDa.

In accordance with the various embodiments described herein, the biopolymer matrix of the implantable macroporous scaffolds of the present disclosure can comprise alginate having a G/M ratio from about 0.5 to about 5.0. In some embodiments, the biopolymer matrix comprises an alginate matrix having a G/M ratio from about 0.5 to about 4.0. In some embodiment, the biopolymer matrix comprises an alginate matrix having a G/M ratio from about 0.5 to about 3.0. In some embodiment, the biopolymer matrix comprises an alginate matrix having a G/M ratio from about 0.5 to about 2.0. In some embodiment, the biopolymer matrix comprises an alginate matrix having a G/M ratio from about 0.5 to about 1.0. In some embodiment, the biopolymer matrix comprises an alginate matrix having a G/M ratio from about 1.0 to about 5.0. In some embodiment, biopolymer matrix comprises an alginate matrix having a G/M ratio from about 2.0 to about 5.0. In some embodiment, the biopolymer matrix comprises an alginate matrix having a G/M ratio from about 3.0 to about 5.0. In some embodiment, the biopolymer matrix comprises an alginate matrix having a G/M ratio from about 4.0 to about 5.0. In some embodiment, the biopolymer matrix comprises an alginate matrix having a G/M ratio from about 1.0 to about 4.0. In some embodiment, the biopolymer matrix comprises an alginate matrix having a G/M ratio from about 2.0 to about 3.0.

In accordance with the various embodiments described herein, the biopolymer matrix of the implantable macroporous scaffolds of the present disclosure can comprise alginate at a concentration ranging from about 0.1% to about 5.0% (w/v). In some embodiments, the biopolymer matrix comprises alginate at a concentration ranging from about 0.1% to about 4.5% (w/v). In some embodiments, the biopolymer matrix comprises alginate at a concentration ranging from about 0.1% to about 4.0% (w/v). In some embodiments, the biopolymer matrix comprises alginate at a concentration ranging from about 0.1% to about 3.5% (w/v). In some embodiments, the biopolymer matrix comprises alginate at a concentration ranging from about 0.1% to about 3.0% (w/v). In some embodiments, the biopolymer matrix comprises alginate at a concentration ranging from about 0.1% to about 2.5% (w/v). In some embodiments, the biopolymer matrix comprises alginate at a concentration ranging from about 0.1% to about 2.0% (w/v). In some embodiments, the biopolymer matrix comprises alginate at a concentration ranging from about 0.1% to about 1.5% (w/v). In some embodiments, the biopolymer matrix comprises alginate at a concentration ranging from about 0.1% to about 1.0% (w/v). In some embodiments, the biopolymer matrix comprises alginate at a concentration ranging from about 0.1% to about 0.5% (w/v). In some embodiments, the biopolymer matrix comprises alginate at a concentration ranging from about 0.5% to about 5.0% (w/v). In some embodiments, the biopolymer matrix comprises alginate at a concentration ranging from about 1.5% to about 5.0% (w/v). In some embodiments, the biopolymer matrix comprises alginate at a concentration ranging from about 2.0% to about 5.0% (w/v). In some embodiments, the biopolymer matrix comprises alginate at a concentration ranging from about 2.5% to about 5.0% (w/v). In some embodiments, the biopolymer matrix comprises alginate at a concentration ranging from about 3.0% to about 5.0% (w/v). In some embodiments, the biopolymer matrix comprises alginate at a concentration ranging from about 3.5% to about 5.0% (w/v). In some embodiments, the biopolymer matrix comprises alginate at a concentration ranging from about 4.0% to about 5.0% (w/v). In some embodiments, the biopolymer matrix comprises alginate at a concentration ranging from about 4.5% to about 5.0% (w/v). In some embodiments, the biopolymer matrix comprises alginate at a concentration ranging from about 1.5% to about 3.5% (w/v). In some embodiments, the biopolymer matrix comprises alginate at a concentration ranging from about 2.0% to about 4.0% (w/v). In some embodiments, the biopolymer matrix comprises alginate at a concentration ranging from about 3.0% to about 4.0% (w/v). In some embodiments, the biopolymer matrix comprises alginate at a concentration ranging from about 0.5% to about 2.0% (w/v). In some embodiments, the biopolymer matrix comprises alginate at a concentration ranging from about 0.5% to about 1.5% (w/v). In some embodiments, the biopolymer matrix comprises alginate at a concentration ranging from about 0.5% to about 1.0% (w/v). In some embodiments, the biopolymer matrix comprises alginate at a concentration ranging from about 1.0% to about 2.0% (w/v). In some embodiments, the biopolymer matrix comprises alginate at a concentration ranging from about 1.0% to about 1.5% (w/v). In some embodiments, the biopolymer matrix comprises alginate at a concentration ranging from about 1.5% to about 2.0% (w/v).

In accordance with the various embodiments described herein, the biopolymer matrix of the implantable macroporous scaffolds of the present disclosure can comprise calcium alginate having a calcium concentration ranging from about 0.1% to about 1.0% (w/v). In some embodiments, the biopolymer matrix comprises a calcium concentration ranging from about 0.1% to about 0.9% (w/v). In some embodiments, the biopolymer matrix comprises a calcium concentration ranging from about 0.1% to about 0.8% (w/v). In some embodiments, the biopolymer matrix comprises a calcium concentration ranging from about 0.1% to about 0.7% (w/v). In some embodiments, the biopolymer matrix comprises a calcium concentration ranging from about 0.1% to about 0.6% (w/v). In some embodiments, the biopolymer matrix comprises a calcium concentration ranging from about 0.1% to about 0.5% (w/v). In some embodiments, the biopolymer matrix comprises a calcium concentration ranging from about 0.1% to about 0.4% (w/v). In some embodiments, the biopolymer matrix comprises a calcium concentration ranging from about 0.1% to about 0.3% (w/v). In some embodiments, the biopolymer matrix comprises a calcium concentration ranging from about 0.1% to about 0.2% (w/v). In some embodiments, the biopolymer matrix comprises a calcium concentration ranging from about 0.2% to about 0.9% (w/v). In some embodiments, the biopolymer matrix comprises a calcium concentration ranging from about 0.3% to about 0.9% (w/v). In some embodiments, the biopolymer matrix comprises a calcium concentration ranging from about 0.4% to about 0.9% (w/v). In some embodiments, the biopolymer matrix comprises a calcium concentration ranging from about 0.5% to about 0.9% (w/v). In some embodiments, the biopolymer matrix comprises a calcium concentration ranging from about 0.6% to about 0.9% (w/v). In some embodiments, the biopolymer matrix comprises a calcium concentration ranging from about 0.7% to about 0.9% (w/v). In some embodiments, the biopolymer matrix comprises a calcium concentration ranging from about 0.8% to about 0.9% (w/v). In some embodiments, the biopolymer matrix comprises a calcium concentration ranging from about 0.2% to about 0.8% (w/v). In some embodiments, the biopolymer matrix comprises a calcium concentration ranging from about 0.3% to about 0.6% (w/v). In some embodiments, the biopolymer matrix comprises a calcium concentration ranging from about 0.4% to about 0.8% (w/v). In some embodiments, the biopolymer matrix comprises a calcium concentration ranging from about 0.3% to about 0.7% (w/v). In some embodiments, the biopolymer matrix comprises a calcium concentration ranging from about 0.2% to about 0.5% (w/v). In some embodiments, the biopolymer matrix comprises a calcium concentration ranging from about 0.3% to about 0.7% (w/v).

Embodiments of the present disclosure also include methods of making any of the macroporous scaffolds disclosed herein. In accordance with these embodiments, the methods include crosslinking alginate strands using calcium to form an alginate hydrogel; cryogelating the hydrogel to form an alginate cryogel; lyophilizing the cryogel to form a macroporous scaffold; mixing retrovirus and freshly isolated immune cells; and seeding the retrovirus and immune cell mixture onto the macroporous scaffolds. In some embodiments, the method comprises activating immune cells (for example, activation with an anti-CD3 antibody and/or an anti-CD28 antibody) prior to mixing with retrovirus. In some embodiments, the method further comprises adding a biological agent (e.g., a cytokine (e.g., IL-2)) to the macroporous scaffold before or contiguous with seeding the scaffold with the immune cells and retrovirus.

In some embodiments, the macroporous scaffolds of the present disclosure can be hydrated by adding a composition (e.g., an aqueous sample) comprising a plurality of cells to be transduced. In some embodiments, the composition also comprises the transduction agent (e.g., viral vector comprising the nucleic acid cargo) to be transduced into the plurality of cells. Prior to addition of this composition, which hydrates the scaffold, the scaffold can be a dry macroporous scaffold (e.g., as described in PCT/US2021/026805, filed Apr. 12, 2021 (published as International WO 2021/207724) and U.S. Ser. No. 17/917,770, each of which is hereby incorporated by reference in its entirety).

In accordance with these embodiments, the biopolymer matrix is generated at a temperature ranging from about 0° C. to about −80° C. In some embodiments, the biopolymer matrix is generated at a temperature ranging from about 0° C. to about −70° C. In some embodiments, the biopolymer matrix is generated at a temperature ranging from about 0° C. to about −60° C. In some embodiments, the biopolymer matrix is generated at a temperature ranging from about 0° C. to about −50° C. In some embodiments, the biopolymer matrix is generated at a temperature ranging from about 0° C. to about −40° C. In some embodiments, the biopolymer matrix is generated at a temperature ranging from about 0° C. to about −30° C. In some embodiments, the biopolymer matrix is generated at a temperature ranging from about 0° C. to about −20° C. In some embodiments, the biopolymer matrix is generated at a temperature ranging from about 0° C. to about −10° C. In some embodiments, the biopolymer matrix is generated at a temperature ranging from about −10° C. to about −80° C. In some embodiments, the biopolymer matrix is generated at a temperature ranging from about −20° C. to about −80° C. In some embodiments, the biopolymer matrix is generated at a temperature ranging from about −30° C. to about −80° C. In some embodiments, the biopolymer matrix is generated at a temperature ranging from about −40° C. to about −80° C. In some embodiments, the biopolymer matrix is generated at a temperature ranging from about −50° C. to about −80° C. In some embodiments, the biopolymer matrix is generated at a temperature ranging from about −60° C. to about −80° C. In some embodiments, the biopolymer matrix is generated at a temperature ranging from about −70° C. to about −80° C. In some embodiments, the biopolymer matrix is generated at a temperature ranging from about −10° C. to about −70° C. In some embodiments, the biopolymer matrix is generated at a temperature ranging from about −20° C. to about −60° C. In some embodiments, the biopolymer matrix is generated at a temperature ranging from about −30° C. to about −50° C. In some embodiments, the biopolymer matrix is generated at a temperature ranging from about −40° C. to about −60° C.

In some embodiments, the biopolymer matrix making up the macroporous scaffold can be a “dry scaffold.” As used herein, “dry scaffold” refers to any scaffold having no more than 10% water by mass. In other words, the dry scaffold has less than 20% water by mass. In some embodiments, the dry scaffold comprises no more than about 18% water by mass. In some embodiments, the dry scaffold comprises no more than about 16% water by mass. In some embodiments, the dry scaffold comprises no more than about 14% water by mass. In some embodiments, the dry scaffold comprises no more than about 12% water by mass. In some embodiments, the dry scaffold comprises no more than about 10% water by mass. In some embodiments, the dry scaffold comprises no more than about 8% water by mass. In some embodiments, the dry scaffold comprises no more than about 6% water by mass. In some embodiments, the dry scaffold comprises no more than about 4% water by mass. In some embodiments, the dry scaffold comprises no more than about 2% water by mass. In some embodiments, the dry scaffold comprises from about 5% to about 15% water by mass. In some embodiments, the dry scaffold comprises from about 10% to about 15% water by mass. In some embodiments, the dry scaffold comprises from about 15% to about 20% water by mass. In some embodiments, the dry scaffold comprises from about 5% to about 10% water by mass. In some embodiments, the dry scaffold comprises from about 10% to about 20% water by mass.

In some embodiments, the dry scaffold comprises no more than about 1.0% of a crosslinking agent (e.g., by weight). This includes embodiments of the dry scaffold that comprise no detectable crosslinking agent. In some embodiments, the dry scaffold comprises no more than about 0.9% of a crosslinking agent. In some embodiments, the dry scaffold comprises no more than about 0.8% of a crosslinking agent. In some embodiments, the dry scaffold comprises no more than about 0.7% of a crosslinking agent. In some embodiments, the dry scaffold comprises no more than about 0.6% of a crosslinking agent. In some embodiments, the dry scaffold comprises no more than about 0.5% of a crosslinking agent. In some embodiments, the dry scaffold comprises no more than about 0.4% of a crosslinking agent. In some embodiments, the dry scaffold comprises no more than about 0.3% of a crosslinking agent. In some embodiments, the dry scaffold comprises no more than about 0.2% of a crosslinking agent. In some embodiments, the dry scaffold comprises no more than about 0.1% of a crosslinking agent. In some embodiments, the dry scaffold comprises less than about 0.1% of a crosslinking agent. In some embodiments, the dry scaffold comprises from about 0.1% to about 1.0% of a crosslinking agent. In some embodiments, the dry scaffold comprises from about 0.2% to about 0.8% of a crosslinking agent. In some embodiments, the dry scaffold comprises from about 0.4% to about 0.6% of a crosslinking agent. In some embodiments, the dry scaffold comprises from about 0.001% to about 0.1% of a crosslinking agent. In some embodiments, the dry scaffold comprises from about 0.01% to about 0.1% of a crosslinking agent.

In some embodiments, the implantable macroporous scaffolds of the present disclosure can be configured to be a variety of geometric shapes and sizes (e.g., discs, beads, pellets), niches, planar layers (e.g., thin sheets). For example, discs of about 0.1 millimeters to about 50 centimeters in diameter, e.g., 0.1, 0.2, 0.3, 0.4, 0.5, 0.6, 0.7, 0.8, 0.9, 1, 2, 3, 4, 5, 10, 15, 20, 25, 30, 35, 40, 45, 50, 60, 70, 80, 90 millimeters 10, 15, 20, 25, 30, 35, 40, 45, 50 centimeters in diameter can be generated and implanted. The disc may have a thickness of 0.1 to 10 millimeters, e.g., 1, 2, 5 millimeters. The discs can be compressed and/or lyophilized for implantation in a subject. Multicomponent devices, e.g., containing scaffolds, are optionally constructed in concentric layers each of which is characterized by different physical qualities (% polymer, % crosslinking of polymer, chemical composition of the dry scaffold, pore size, porosity, and pore architecture, stiffness, toughness, ductility, viscoelasticity, and/or pharmaceutical composition).

The macroporous scaffolds disclosed herein can be fabricated in or inserted into wells of a multi-well plate, including in 384-well, 96-well, 48-well, 24-well, 12-well, or 6-well plates. The scaffolds can be fabricated in or inserted into flasks, including T25, T75, T175, or T225 flasks. The scaffolds can be fabricated in or inserted into culture dishes, including 35 mm, 60 mm, 100 mm, and 150 mm dishes. Scaffolds can be fabricated in or inserted into cell culture tubes, including 3 mL, 5 mL, 7 mL, 8 mL 12 mL, 14 mL, 15 mL, 16 mL, 19 mL, 21 mL, or 50 mL capacity tubes. The scaffolds can be of arbitrary shape and size and fabricated in or inserted into molds, including molds of size in the range of 1 mm3 to 0.1m3. The scaffolds can be fabricated in or inserted into cell culture bags of capacity 50 mL, 100 mL, 200 mL, 300 mL, 500 mL, 1000 mL, 2000 mL, 5000 mL, 10000 mL.

The scaffolds disclosed herein can be fabricated in wells of multi-well plates or in culture dishes and so have a disk shape with diameter between 1 mm and 50 cm and thickness 1 mm to 50 cm. The scaffolds can be fabricated in square or rectangular mold with side length 1 mm to 50 cm and thickness 1 mm to 50 cm. Additionally, the scaffold can be fabricated in molds that are regularly shared or irregularly shaped molds and have regular including triangular, pentagonal, hexagonal, star shaped, or diamond shaped or they can be irregularly shaped. Regular or irregular shaped molds can have a surface area of between 1 mm2 to 2500 cm² and thickness of 1 mm to 50 cm.

The scaffolds can consist of a collection of individual particles. These microparticles can be manufactured through spray-drying, electrospinning, extrusion, emulsification/gelation, shredding, spin drying or other techniques known to make particles. An example of particles that make up the scaffolds could be microspheres that are 50 μm, 100 μm, 200 μm, 500 μm, 1000 μm, 2000 μm or 5000 μm in diameter. The scaffolds can also consist of sections cut from a larger manufactured whole. The original whole can be 0.1 meter square to 1,000 meter square in size.

The scaffold structure may contain pores which are microporous or macroporous. Pore size may include 10 μm, 20 μm, 50 μm, 100 μm, 200 μm, 500 μm, 1000 μm. In one aspect, 50-70% of the pores comprise a 100-200 mm diameter (for example, scaffolds with 60% of the pores comprise a 100-200 mm diameter). The pattern of the pores is optionally homogeneous, heterogenous, aligned, repeating, or random. In some embodiments, the 100-200 mm diameter pores account for at least 75, 76, 77, 78, 79, 80, 81, 82, 83, 84, 85, 86, 87, 88, 89, 90, 91, 92, 93, 94, or 95% (such as, for example, 82.78%) scaffold volume.

Embodiments of the present disclosure also methods and compositions related to implantable macroporous scaffolds that include at least one biological agent. The biological agent can be included in the compositions of the present disclosure and/or integrated into the biopolymer matrix of the scaffolds. In some embodiments, the biological agent is included to facilitate the growth, proliferation, survival, and/or differentiation of a plurality of cells. The biological agent can be included to enhance compatibility of the plurality of cells and/or the scaffold with host target tissue. The biological agent can also be included to enhance one or more mechanical properties of the scaffold.

In some embodiments, the biological agent is a small molecule. In some embodiments, the small molecule is selected from the group consisting of a TLR agonist, a checkpoint inhibitor, an IDO inhibitor, a MEK inhibitor, an HDAC inhibitor, a PI3K inhibitor, an immunomodulatory drug, a JAK kinase inhibitor, and an mTOR inhibitor. In some embodiments, the at least one biological agent is a protein, peptide, or polypeptide. In some embodiments, the protein, peptide, or polypeptide is selected from the group consisting of a cytokine, an antibody, and a growth factor. In some embodiments, the cytokine comprises at least one of IL-2, IL-15, IL-7, IL-23, TNF-α, and/or IFN-γ.

In some embodiments, the compositions applied to the implantable macroporous scaffolds can further comprise receptors (epidermal growth factor receptor (EGFR), platelet derived growth factor receptor (PDGFR)), ligands (including, but not limited to epidermal growth factor (EGF), platelet derived growth factor, granulocyte macrophage colony-stimulating factor (GM-CSF), vascular endothelial growth factor (VEGF), granulocyte colony-stimulating factor (G-CSF), Granulocyte macrophage colony-stimulating factor (GM-CSF), macrophage colony-stimulating factor (M-CSF), fibroblast growth factor (FGF), insulin-like growth factor (IGF) 1 (IGF-1), and/or IGF-2), bone morphogenic protein (BMP), ephrin (A1, A2, A3, A4, A5, B1, B2, B3), erythropoietin, fibroblast growth factor (FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23), Glial cell line-derived neurotrophic factor (GDNF), Hepatocyte growth factor (HGF), Neurotrophins (BDNF, NGF, NT-3, NT-4), T-cell growth factor (TCGF), Transforming growth factor (TGF-α, TGF-β), Tumor necrosis factor-alpha (TNF-α), Wnt Signaling Pathway, integrins (including VLA-1, VLA-2, VLA-3, VLA-4, VLA-5, VLA-6, FLJ25220, RLC, PRO827, HsT18964, FLJ39841, HUMINAE, LFA1A, MAC-1, VNRA, MSK8, GPIIb, cadherins (for example, E-cadherin), and/or an immune activating and/or sustaining antibodies, chemokines, and cytokines (such as, for example, IL-2, IL-4, IL-6, IL-7, IL-12, IL-15, IL-21, IL-23, TNF-α, or IFN-γ.

As would be understood by one of ordinary skill in the art based on the present disclosure, to fully activate an immune cell, a co-stimulatory signal may be needed. Thus, in one embodiment, the compositions include one or more co-stimulatory molecules which activate a T cell, natural killer (NK) cell, NK T cell, macrophage, tumor infiltrating lymphocyte (TIL), tumor infiltrating NK cell (TINK), or a marrow infiltrating lymphocyte (MIL) including, but not limited to anti-CD28, B7-1, B7-2, anti-inducible costimulator (ICOS), ICOS ligand, anti-CD27, CD70, 4-1BBL, anti-41-BB, anti-CD40L, CD40, anti-DAP10, anti-CD30, CD30L, anti-TIM-1, anti-TIM-2, anti-TIM-3, anti-CD44, anti-NK1.1, lectin like transcript-1 (LLT-1), anti-CD137, CD48, MICA, anti-2B4, and anti-glucocorticoid-induced tumor necrosis factor receptor related protein (GITR). Thus, the compositions applied to the implantable macroporous scaffolds described herein can also include a ligand or antibody that induces signaling through a T cell, NK cell, or NK T cell co-stimulatory receptor including, but not limited to anti-CD28, B7-1, B7-2, anti-inducible costimulator (ICOS), ICOS ligand, anti-CD27, CD70, 4-1BBL, anti-41-BB, anti-CD40L, CD40, anti-DAP10, anti-CD30, CD30L, anti-TIM-1, anti-TIM-2, anti-TIM-3, anti-CD44, anti-NK1.1, lectin like transcript-1 (LLT-1), anti-CD137, CD48, MICA, anti-2B4, and anti-glucocorticoid-induced tumor necrosis factor receptor related protein (GITR).

Embodiments of the present disclosure also include applying compositions comprising a plurality of cells and at least one transduction agent to the biopolymer matrix to generate the scaffolds described herein. In accordance with these embodiments, the plurality of cells can include one or more immune cells. In some embodiments, the one or more immune cells are selected from the group consisting of a T cell, a B cell, a natural killer (NK) cell, an NK T cell, a macrophage, a dendritic cell, a tumor infiltrating lymphocyte (TIL), a tumor infiltrating NK cell (TINK), and a marrow infiltrating lymphocyte (MIL).

In some embodiments, the target cell is a commercial (e.g., immortal) cell line or a primary cell line. In some embodiments, the target cell is an immune cell. In some embodiments, the immune cell is selected from the group consisting of a T cell, a natural killer (NK) cell, an NK T cell, a macrophage, a tumor infiltrating lymphocyte (TIL), a tumor infiltrating NK cell (TINK), and a marrow infiltrating lymphocyte (MIL). In some embodiments, the cell is a stem cells. In some embodiments the cell is a hematopoetic stem cells. In some embodiments, the cell is an engineered cell. In certain embodiments, the engineered cell includes, but is not limited to, tissue sources derived from adipose tissue, skin tissue, muscle tissue, blood, bone marrow, nerve tissue, liver tissue, pancreatic tissue, cartilage tissue, lung tissue, intestinal tissue, ovarian tissue, testicular tissue, umbilical cord tissue, placental tissue, synthetic and biomimetic scaffolds, and any derivatives thereof. In some embodiments, the cell can be any cell type. In certain embodiments, the cell type can be prokaryotic cells, eukaryotic cells, human-specific cells, immune cells, stem cells, cancer cells, microbial cells, specialized cells, and any derivatives thereof. In some embodiments, the cell can be any cell type. In certain embodiments, the cell type can be prokaryotic cells, eukaryotic cells, human-specific cells, immune cells, stem cells, cancer cells, microbial cells, specialized cells, and any derivatives thereof.

In some embodiments, the transducing agent is a vector, which is used to deliver a target nucleic acid to a cell. In some embodiments, the vector is selected from the group consisting of a lentivirus, a retrovirus, an adenovirus, herpes simplex virus (HSV), vesicular stomatitis virus (VSV), Sendai virus, and an adeno-associated virus, cocal virus, baculovirus. In some embodiments, the transducing agent comprises a virus-like particle, a cell-mimicking particle, a transposon, an exosome, a nanoparticle, a micelle, a liposome, a Modified Vaccinia Ankara (MVA), a plasmid, and any derivatives thereof.

As described further herein, embodiments of the present disclosure facilitate the delivery of a target nucleic acid to a cell. As would be recognized by one of ordinary skill in the art based on the present disclosure, the target nucleic acid can be any nucleic acid. In some embodiments, the target nucleic acid comprises RNA. For example, the target nucleic acid can be siRNA, tasiRNA, lncRNA, shRNA, mRNA, gRNA, miRNA, and viral RNA including any combinations and/or derivatives thereof. In other embodiments, the target nucleic acid comprises DNA, including any derivatives or variants thereof. In some embodiments, the target nucleic acid is DNA that encodes an RNA. In some embodiments, the RNA encoded by the DNA is siRNA, tasiRNA, lncRNA, shRNA, mRNA, gRNA, miRNA, and viral RNA. In some embodiments, the target nucleic encodes a protein (e.g., a chimeric antigen receptor, or CAR).

In some embodiments, the dry scaffold comprises at least one biological agent. In some embodiments, the at least one biological agent is a small molecule. In some embodiments, the small molecule includes, but is not limited to, TLR agonists, checkpoint inhibitors, IDO inhibitors, MEK inhibitors, HDAC inhibitors, PI3K inhibitors, immunomodulatory drugs, JAK, and mTOR inhibitors, including any combinations thereof. In some embodiments, the at least one biological agent is a protein, peptide, or polypeptide. In some embodiments, the protein, peptide, or polypeptide includes, but is not limited to, a cytokine, an antibody, and a growth factor, including any combinations thereof.

As described further herein, the present disclosure provides methods of transducing a cell (e.g., an immune cell) using the implantable macroporous scaffolds of the present disclosure. In some embodiment, the cells can include, for example, T cells, B cells, natural killer (NK) cells, NK T cells, macrophages, tumor infiltrating lymphocytes (TILs), tumor infiltrating NK cells (TINKs), or marrow infiltrating lymphocytes (MILs), or any combination thereof. In some embodiments, the one or more cells are obtained from an autologous, allogeneic, and/or haplo-identical donor source. In some embodiments, the one or more cells include a non-immune cell (such as, for example, a mesenchymal stem cell (MSC), hematopoietic stem cell (HSC), dendritic cell, neural stem cell, induced pluripotent stem cell, or islet cells). In some embodiments, the one or more cells include immune and non-immune primary cells and cell lines.

In some embodiments, the implantable macroporous scaffolds of the present disclosure facilitate the transduction of a plurality of cells with a transduction agent, such as a viral vector (such as, for example, lentivirus, retrovirus, adenovirus, adeno-associated virus, virus-like particle, transposon, or liposome) encoding a therapeutic cargo (including, but not limited, a polynucleotide encoding a fusion protein, a chimeric antigen receptor (CAR (e.g., a CAR T cell, CAR NK cell, CAR NK T cell, or CAR macrophage that targets CD19, CD33, IL-13 receptor α chain 2 (IL13Rα2), B7-H3, neural/glial antigen 2 (NG2), disialoganglioside GD2, EGFRvIII, MUC1, PSMA, mesothelin, HER2, or CEA)), an exogenous gene, siRNA, tasiRNA, lncRNA, shRNA, mRNA, gRNA, miRNA, and/or DNA encoding said gene, a therapeutic ligand, or a combination thereof) to treat a disease or disorder in a target tissue.

In some embodiments, the compositions comprising a plurality of cells and a transduction agent are applied to the macroporous scaffolds of the present disclosure to facilitate transduction of the cells with a therapeutic nucleic acid molecule for a certain incubation period prior to administration/implantation in a subject. In some embodiments, the cells are incubated with the scaffold for at least 1, 2, 3, 4, 5, 10, 15, 20, 25, 30, 35, 40, 45, 50, or 55 minutes. In some embodiments, the cells are incubated with the scaffold for at least 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 36, 37, 38, 39, 40, 41, 42, 43, 44, 45, 46, 47, 48, 54, 60, 66, 72, 84, or 96 hours. In some embodiments, the cells are incubated with the scaffold for at least 5, 6, 7, 8, 9, 10, 11, 12, 13, or 14 days. In some embodiments, the compositions comprising a plurality of cells and a transduction agent are applied to the macroporous scaffolds of the present disclosure to facilitate transduction of the cells with a therapeutic nucleic acid molecule without prior incubation (i.e., are implanted into a subject without prior incubation).

As described above, the cells can be a cell from a commercially available cell line. Exemplary cells that can be transduced include, but are not limited to NCI-H295R, 5637, HT-1376, J82, SW 780, T24, T24-Luc-Neo, T24P, BT142, D54-Luc, DBTRG (tumor), DBTRG-05MG, Gli36-DsRed-R-Luc (rescued), LN-18, LN-229, LN-827 (pMMP-LucNeo), M059K, SF-295, SF-539, SF-767, SNB-19, U-251, U-251-Luc-mCh-Puro, U-87 MG, U-87 MG-Luc, Ca Ski, HeLa, KB, C2BBe1, Caco-2, COLO 205, COLO 205-Luc #2, DLD-1, HCC2998, HCT-116, HCT-116-Luc, HCT-15, HCT-8, HT-29, HT-29-Luc, LoVo, LoVo-6-Luc1, LS 174T, LS411N, NCI-H508, SW-480, SW-620, A-431, HEKn, HEL, HEL 92.1.7, HEL 92.1.7-Luc-Neo, HEL-Luc-Neo, TF-la-Luc-Neo, OE33, A4573, Hs 895.T, NHDF (normal human dermal fibroblasts), TE 353.Sk, TE 354.T, GIST-T1, NCI-N87, NUGC-4, SNU-5, CAL 27, FaDu, L1210, M-NFS-60, HL-60, EOL-1, Kasumi-1, Kasumi-3, Kasumi-3-Luc-mCh-Puro, KG-1-Luc-mCh-Puro, MOLM-13, MV-4-11, MV-4-11-Luc-mCh-Puro, NOMO-1, THP-1, NALM6, NALM6-Luc-MCh-Puro, Rch, Rch (pMMP-Luc-Neo), K-562, K-562-Luc2, ARH-77, CCRF-CEM, DND-41-Luc-mCh-Puro, Jurkat, Jurkat-Clone E6-1, MOLT-4, MOLT-4-Luc-MCh-Puro, Hep 3B2.1-7, Hep G2, LL, LL/2, LL/2-Luc-M38, NCI-H596, Calu-6, NCI-H322M, A549, A549-Luc-C8, Calu-1, Calu-3, HCC827, HCC827-Luc-mCh-Puro, NCI-H125, NCI-H125-Luc, NCI-H1299, NCI-H1650, NCI-H1703, NCI-H1703-Luc-mCh-Puro, NCI-H1975, NCI-H1975-Luc, NCI-H2110, NCI-H2122, NCI-H23, NCI-H292, NCI-H3122, NCI-H441, NCI-H460, NCI-H460-Luc2, NCI-H522, PC-9, DMS 114, NCI-H446, NCI-H69, NCI-H82, SHP-77, EBC-1, SK-MES-1, RL, DB, DB/M2, GRANTA-519, Farange, B-JAB, Daudi, Daudi-Luc-mCh-Puro, NAMALWA, Raji, Raji-Luc, Ramos, Ramos-Luc, HuT78, HT, SU-DHL-6, SU-DHL-6-Luc-mCh-Puro, OCI-Ly1 LN, OCI-Ly19-Luc-Nco, OCI-Ly3-Luc-mCh-Puro, OCI-Ly7-Luc-mCh-Puro (rescued), OCI-Ly7-Luc-Neo, Pfeiffer, SU-DHL-10, SU-DHL-10-LN-High, SU-DHL-16, SU-DHL-4-Luc-mCh-Puro, SU-DHL-8, TMD8, Toledo-Luc-Neo, WSU-DLCL2, WSU-FSCCL, WSU-FSCCL-CMV-Luc-Puro, WSU-FSCCL-MSCV-Luc-Puro-copGFP, NK-92 MI, KARPAS 299, BT-20, BT-474, HCC1395, HCC70, Hs 578Bst, Hs 578T, MCF 10A, MCF-7, MCF7-Luc-mCh-Puro, MDA-MB-231, MDA-MB-231-2LMP, MDA-MB-231-Luc-D3H1, MDA-MB-231-Luc-D3H2, MDA-MB-231-Luc-D3H2LN, MDA-MB-231-Luc-D3H3, MDA-MB-361, MDA-MB-453, MDA-MB-468, MX-1, MX-1-Luc, SK-BR-3, T47D, UISO-BCA-1, ZR-75-1, A2058, A375, COLO 829, G-361, LOX-IMVI, M14, MDA-MB-435S, OCM-1, OCM-1-Luc-mCh-Puro, PA-NUT, SK-MEL-28, SK-MEL-28-Luc-mCh-Puro, SK-MEL-5, UACC-62, WM-115, WM-266-4, JJN-3-Luc, MM.1S (pMMP-Luc-Neo), NCI-H929, NCI-H929-Luc-mCh-Puro, OPM-2, RPMI 8226, U266B1, SK-N-AS, SK-N-FI, SK-N-SH, MKL-1, A2780, A2780-Luc, IGROV1, IGROV1-Luc-Mch-Puro, OVCAR-4, OVCAR-5, OVCAR-5-Luc-mCh-Puro, OVCAR-8, OVCAR-8-Luc-mCh-Puro, SK-OV-3 (Subcutaneous), SK-OV-3-Luc-D3 (Intraperitoneal), Bx-PC-3, BxPC-3-Luc2, Capan-1, Capan-2, KP4, MIA PaCa-2, MIA PaCa-2-Luc, PANC-1, PANC-1-Luc, SU-86.86, SW 1990, 22Rv1, CWR-22-R, DU 145, DU 145-Luc, LnCap, LnCap clone FGC, PC-3, PC-3-Luc, PC-3M-Luc-C6 (Intracardiac), PC-3M-Luc-C6 (Orthotopic), PC-3M-Luc-C6 (Peritibial), PC-3M-Luc-C6 (SC-Axilla), VCaP, 769-P, 786-O, 786-O-Luc-Neo (rescued), A-498, ACHN, Caki-1, TK-10, A-673, HT-1080, MG-63, Saos-2, SJSA-1, SW 872, MB-1, TT, SK-LMS-1, 293T, HEK293, or HeLa cells. It is also understood and herein contemplated that the transduced cell can be an adherent (e.g., HEK cells) or nonadherent cell (e.g., T cells) or cell line.

Embodiments of the present disclosure also include applying compositions comprising a plurality of cells and at least one transduction agent to the biopolymer matrix to generate the scaffolds described herein. In accordance with these embodiments, the implantable macroporous scaffolds of the present disclosure facilitate the transduction of a plurality of cells with a transduction agent. Transduction of a cell (e.g., an immune cell) can occur by any means known in the art based on the present disclosure. In some embodiments, transduction of a cell can occur via a vector encoding a transgene (e.g., a CAR). Accordingly, the scaffolds disclosed herein can include a vector, such as, for example, a lentivirus, retrovirus, adenovirus, adeno-associated virus, virus-like-particle, liposome, or transposon, encoding a transgene, such as, for example, a chimeric antigen receptor (CAR).

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, virus-like particles (VLPs), transposons (such as, for example, class II transposable elements comprising Sleeping Beauty transposase, Frog Prince, piggyBac, Tol2 and other Tc1/mariner-type transposases), zinc finger nucleases, meganucleases, transcription activator-like effectors (e.g., TALENs), triplexes, mediators of epigenetic modification, and CRISPR and rAAV technologies), minicircle DNA, or via transfer of genetic material in cells or carriers such as virus-like particle, cell-mimicking particles, transposons, exosomes, nanoparticles, micelles or liposomes. Appropriate means for transfection, including vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

A retrovirus is an animal virus belonging to the virus family of Retroviridae, including any types, subfamilies, genus, or tropisms. Retroviral vectors, in general, are described by Verma, I. M., Retroviral vectors for gene transfer. Examples of retroviruses that can be used as vectors include, but are not limited to, human T-lymphotrophic virus (HTLV)-1 (HTLV-1), HTLV-2, HTLV-3, HTLV-4, simian foamy virus, human foamy virus, simian immunodeficiency virus (SIV), human immunodeficiency virus (HIV), and Rous sarcoma virus. A retrovirus is essentially a package which has packed into it a nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically, a retroviral genome, contains the gag, pol, and env genes which are involved in the making of the protein coat. It is the gag, pol, and env genes which are typically replaced by the foreign DNA that is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. In some embodiments, positive and/or negative selectable markers can be included along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

Lentiviral vectors (LVs), including, but not limited to human immunodeficiency (HIV) vectors and simian immunodeficiency virus (SIV) vectors, are versatile vectors for cell culture or in vivo gene transfer into dividing and nondividing cells. This system has the advantage of being flexible for transducing a range of lung cancer cells, without having to spend time selecting for stable expression. Replication defective VSV G-pseudotyped lentivirus vectors (produced on order by GeneCopoeia) can be used to transduce cells.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)). A viral vector can be one based on an adenovirus which has had the E1 gene removed and these virons are generated in a cell line such as the human 293 cell line. In another embodiment, both the E1 and E3 genes are removed from the adenovirus genome.

Another type of viral vector is based on an adeno-associated virus (AAV). This defective parvovirus can be a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property can also be used. Once embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson, Curr Opin Mol Ther 5:633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Promoters controlling transcription from vectors in mammalian host cells may be obtained from various sources, including but not limited to, the genomes of viruses such as: polyoma, Simian Virus 40 (SV40), adenovirus, retroviruses, hepatitis-B virus and cytomegalovirus, or from heterologous mammalian promoters, e.g., beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18:355-360 (1982)). Promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, fetoprotein and insulin), typically one will use an enhancer from a eukaryotic cell virus for general expression. Examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs, the promoter and/or enhancer region to be active in all eukaryotic cell types, even if it is only expressed in a particular type of cell at a particular time. A promoter of this type is the CMV promoter (650 bases). Other promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. In some embodiments, the transcription unit also contains a polyadenylation region. One benefit of this region is that it increases the likelihood that the transcribed unit will be processed and transported like mRNA. The identification and use of polyadenylation signals in expression constructs is well established. Homologous polyadenylation signals can be used in the transgene constructs. In certain transcription units, the polyadenylation region is derived from the SV40 early polyadenylation signal and consists of about 400 bases. The transcribed units can contain other standard sequences alone or in combination with the above sequences improve expression from, or stability of, the construct.

The viral vectors can include nucleic acid sequence encoding a marker product. This marker product is used to determine if the gene has been delivered to the cell and once delivered is being expressed. Marker genes can include the E. coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 3. METHODS OF USE

Embodiments of the present disclosure also include a method of treating a subject. In accordance with these embodiments, and as described further herein, the method includes implanting a macroporous scaffold within or adjacent to a target tissue, wherein the scaffold comprises: a crosslinked biopolymer matrix comprising an average pore size ranging from about 10 μm to about 500 μm, and a stiffness ranging from about 1 kPa to about 1000 kPa, wherein the stiffness of the matrix is compatible with the stiffness of the target tissue; and a composition comprising a plurality of cells and a transduction agent. In some embodiments, the scaffold facilitates transduction of the plurality of cells with the transduction agent, and wherein the transduced cells treat the subject.

In some embodiments of the method, target tissue is tumor tissue. In some embodiments of the method, the target tissue is solid tumor tissue. In some embodiments of the method, the target tissue comprises at least one of lung tissue, bone tissue, skin tissue, breast tissue, muscle tissue, nerve tissue, brain tissue, lymph tissue, prostate tissue, bladder tissue, stomach tissue, intestinal tissue, uterine tissue, ovarian tissue, liver tissue, adipose tissue, cartilaginous tissue, thyroid tissue, and/or pancreatic tissue. In some embodiments of the method, the subject has been diagnosed with a disease or condition. In some embodiments of the method, the disease or condition comprises cancer.

In some embodiments, the scaffold facilitates the transduction of a plurality of cells (e.g., immune cells) with a transduction agent (e.g., viral vector containing a polynucleotide encoding a protein-of-interest) with a transduction efficiency of at least 50% (measured in vivo or ex vivo). In some embodiments, the scaffold facilitates the transduction of the plurality of cells with the transduction agent with a transduction efficiency of at least 60%. In some embodiments, the scaffold facilitates the transduction of the plurality of cells with the transduction agent with a transduction efficiency of at least 70%. In some embodiments, the scaffold facilitates the transduction of the plurality of cells with the transduction agent with a transduction efficiency of at least 80%. In some embodiments, the scaffold facilitates the transduction of the plurality of cells with the transduction agent with a transduction efficiency of at least 90%. In some embodiments, the scaffold facilitates the transduction of the plurality of cells with the transduction agent with a transduction efficiency of about 50% to about 90%. In some embodiments, the scaffold facilitates the transduction of the plurality of cells with the transduction agent with a transduction efficiency of about 60% to about 90%. In some embodiments, the scaffold facilitates the transduction of the plurality of cells with the transduction agent with a transduction efficiency of about 70% to about 90%. In some embodiments, the scaffold facilitates the transduction of the plurality of cells with the transduction agent with a transduction efficiency of about 50% to about 80%. In some embodiments, the scaffold facilitates the transduction of the plurality of cells with the transduction agent with a transduction efficiency of about 50% to about 70%. In some embodiments, the scaffold facilitates the transduction of the plurality of cells with the transduction agent with a transduction efficiency of about 60% to about 80%. In some embodiments, the scaffold facilitates the transduction of the plurality of cells with the transduction agent with a transduction efficiency of about 70% to about 90%.

In some embodiments of the method, the biopolymer matrix comprises at least one of alginate, Hyaluronic acid, collagen, fibrin, Poly Lactic-co-Glycolic Acid (PLGA), Polycaprolactone (PCL), gelatin, Polyethylene glycol (PEG), chitosan, cellulose, polyglutamic acid, fibrin, silk, agarose, dextran, polyacrylamide, polyvinyl alcohol, Poly(N-isopropylacrylamide), Poly(2-hydroxyethyl methacrylate), polyurethane, polyethyleneimine, Poly(methyl methacrylate, Poly(2-oxazoline), Polyphosphazenes, and any composites, derivatives, or combinations thereof.

As described further above, in some embodiments of the method, the biopolymer matrix comprises alginate having a molecular weight from about 1 kDa to about 500 kDa. In some embodiments of the method, the biopolymer matrix comprises alginate having a G/M ratio from about 0.5 to about 5.0. In some embodiments of the method, the biopolymer matrix comprises alginate at a concentration ranging from about 0.1% to about 5.0%. In some embodiments of the method, the biopolymer matrix comprises alginate having a calcium concentration ranging from about 0.1% to about 1.0%. In some embodiments of the method, the biopolymer matrix is generated at a temperature ranging from about −20° C. to about −80° C.

As described further above, in some embodiments of the method, the biopolymer matrix exhibits a stiffness that is from about ±25%, about ±50%, about ±75%, about ±100%, about ±125%, about ±150%, about ±175%, about ±200%, about ±225%, or about ±250% of the stiffness of the target tissue.

In some embodiments of the method, the scaffold comprises at least one biological agent. In some embodiments of the method, the at least one biological agent is a small molecule. In some embodiments of the method, the small molecule is selected from the group consisting of a TLR agonist, a checkpoint inhibitor, an IDO inhibitor, a MEK inhibitor, an HDAC inhibitor, a PI3K inhibitor, an immunomodulatory drug, a JAK kinase inhibitor, and an mTOR inhibitor. In some embodiments of the method, the at least one biological agent is a protein, peptide, or polypeptide. In some embodiments of the method, the protein, peptide, or polypeptide is selected from the group consisting of a cytokine, an antibody, and a growth factor. In some embodiments of the method, the cytokine comprises at least one of IL-2, IL-15, IL-7, IL-23, TNF-α, and/or IFN-γ.

In some embodiments of the method, the plurality of cells comprise one or more immune cells. In some embodiments of the method, the one or more immune cells are selected from the group consisting of a T cell, a B cell, a natural killer (NK) cell, an NK T cell, a macrophage, a dendritic cell, a tumor infiltrating lymphocyte (TIL), a tumor infiltrating NK cell (TINK), and a marrow infiltrating lymphocyte (MIL). In some embodiments of the method, the one or more immune cells are activated. In some embodiments of the method, the plurality of cells are obtained from cell culture. In some embodiments of the method, the plurality of cells are obtained from a donor.

As described further above, in some embodiments of the method, the transduction agent comprises a viral vector. In some embodiments of the method, the viral vector is selected from the group consisting of a lentivirus, a retrovirus, an adenovirus, an adeno-associated virus, a cocal virus, and a baculovirus. As described further above, in some embodiments of the method, the transduction agent comprises a virus-like particle, a cell-mimicking particle, a transposon, an exosome, a nanoparticle, a micelle, and a liposome. In some embodiments of the method, the transduction agent comprises a nucleic acid cargo. In some embodiments of the method, the nucleic acid cargo comprises siRNA, tasiRNA, lncRNA, shRNA, mRNA, gRNA, miRNA, and/or viral RNA. In some embodiments of the method, the nucleic acid cargo comprises DNA that encodes a fusion protein, a chimeric antigen receptor (CAR), a therapeutic peptide or polypeptide, or a combination thereof.

In accordance with the above embodiments, the present disclosure provides various pharmaceutically acceptable embodiments of the implantable macroporous scaffolds disclosed herein. As described above, the scaffolds of the present disclosure can also be administered in vivo with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be understood to one of ordinary skill in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art. Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389.

In accordance with the above embodiments, the scaffolds of the present disclosure can be implanted within or adjacent to the target tissue. In some embodiments, the target tissue is tumor tissue. In some embodiments, the target tissue is solid tumor tissue. In some embodiments, the target tissue comprises at least one of lung tissue, bone tissue, skin tissue, breast tissue, muscle tissue, nerve tissue, brain tissue, lymph tissue, prostate tissue, bladder tissue, stomach tissue, intestinal tissue, uterine tissue, ovarian tissue, liver tissue, adipose tissue, cartilaginous tissue, thyroid tissue, and/or pancreatic tissue.

The disclosed scaffolds can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

In some embodiments, disclosed herein are methods of treating, inhibiting, reducing, decreasing, ameliorating, and/or preventing a solid cancerous tumor and/or metastasis (such as, for example brain cancer including, but not limited to a glioblastoma) in a subject comprising implanting any of the loaded macroporous scaffolds disclosed herein into the subject, wherein the scaffold is implanted in the tissue, and wherein when hydrated with a biological fluid, the scaffold exhibits a Young's modulus compatible with the tissue. For examples, disclosed herein are methods of treating, inhibiting, reducing, decreasing, ameliorating, and/or preventing a solid cancerous tumor and/or metastasis (such as, for example, a brain cancer including, but not limited to a glioblastoma) in a subject comprising implanting the loaded macroporous scaffold comprising a viral vector (such as, for example, lentivirus, retrovirus, adenovirus, adeno-associated virus, virus-like particle, transposon, or liposome) encoding a therapeutic cargo (including, but not limited to a fusion protein, a chimeric antigen receptor (CAR) (such as, for example, a CAR T cell, CAR NK cell, CAR NK T cell, or CAR macrophage that targets CD19, CD33, IL-13 receptor α chain 2 (IL13Rα2), B7-H3, neural/glial antigen 2 (NG2), disialoganglioside GD2, epidermal growth factor receptor vIII (EGFRVIII), MUC1, PSMA, mesothelin, HER2, or CEA), an exogenous gene, siRNA, tasiRNA, lncRNA, shRNA, mRNA, gRNA, miRNA, and/or DNA encoding said gene, a therapeutic ligand, or a combination thereof) to treat a disease or disorder in a tissue, wherein the loaded macroporous scaffold is prepared by a process that comprises incubating a dry macroporous scaffold having an average pore size ranging from about 10 μm to about 500 μm, and a stiffness ranging from about 1 kPa to about 1000 kPa.

Also, disclosed herein are methods of treating, inhibiting, reducing, decreasing, ameliorating, and/or preventing a cancerous tumor and/or metastasis using the implantable macroporous scaffolds of the present disclosure. In some embodiments, the scaffold exhibits a stiffness from ±25%, ±50%, ±75%, ±100%, ±125%, ±150%, ±175%, ±200%, ±225%, or ±250% of the stiffness of the tissue (e.g., a cancerous tumor).

As described further herein, generating a scaffold such that it is compatible with a target tissue allows for the scaffold to be directly applied to any target tissue or tumor as opposed to only administering cells transduced in the scaffold. Accordingly, disclosed herein are methods of treating, inhibiting, reducing, decreasing, ameliorating, and/or preventing a cancerous tumor (e.g., a solid tumor) and/or metastasis, wherein the scaffold is implanted subcutaneously or directly on the tumor.

In one embodiment, a CAR can be tailored to target particular cancer by adjusting the target to which the CAR binds. For example, where the cancer is a glioblastoma, the scaffold can comprise one or more CARs that target epidermal growth factor receptor vIII (EGFRvIII), HER2, IL-13 receptor α chain 2 (IL13Rα2), B7-H3, disialoganglioside GD2, and/or MUC1; where the cancer is a leukemia, B-cell acute leukemia, B-cell non-Hodgkins lymphoma, follicular lymphoma, mantel cell lymphoma the scaffold can comprise one or more CARs that target CD20, CD22, and/or CD19 (e.g., Tisagenlecleucel, Axicabtagene ciloleucel, CTL-119, UCART119, JCAR014, JCAR017); where the cancer is multiple myeloma, the scaffold can comprise one or more CARs that target BCMA and/or CD138; where the cancer is a breast cancer, the scaffold can comprise one or more CARs that target MET, MUC1, and/or HER2; wherein the cancer is an ovarian or cervical cancer the scaffold can comprise one or more CARs that target MUC16 and/or FR; wherein the cancer is a pancreatic cancer the scaffold can comprise one or more CARs that target Mucin 1 (MUC1), Mesothelin (MSLN), and/or CD19; wherein the cancer is a prostate cancer the scaffold can comprise one or more CARs that target PSA and/or PSMA, wherein the cancer is acute myeloid leukemia, the scaffold can comprise one or more CARs that target CD33 and/or CD123. Alternatively, the scaffold can comprise a CAR that targets PD-L1, CLDN6, or non-car cargos such as a PD1-CD28 fusion.

In one embodiment, the implantable macroporous scaffolds of the present disclosure include a viral vector (such as, for example, lentivirus, retrovirus, adenovirus, adeno-associated virus, virus-like particle, transposon, or liposome) encoding a therapeutic cargo (including, but not limited to a fusion protein, a chimeric antigen receptor (CAR) (such as, for example, a CAR T cell, CAR NK cell, CAR NK T cell, or CAR macrophage that targets CD19, CD30, CD20, Cd171, Cd80/86, c-MET, DLL-3, DR5, EpHA2, BCMA, GD2, B7H3, NKR2, NKG2D, CD133, CEA, EGFR, EGFR 806, Mesothelin, PSCA, PSMA, EpCAM, MUC1, ICAM-1, CD147, EpHA2, HER2, IL13Rα2, FOLR1, MSLN, CLDN 18.2, VEGFR2, AFP, Nectin4/FAP, Lewis Y, glypican-3, AFP, AXL, DR5, gp100, MAGE-A1/3/4, LMP1, DLL-3, IL-13 receptor α chain 2 (IL13Rα2), B7-H3, neural/glial antigen 2 (NG2), disialoganglioside GD2, epidermal growth factor receptor VIII (EGFRvIII), MUC1, PSMA, mesothelin, HER2, or CEA), an exogenous gene, siRNA, tasiRNA, lncRNA, shRNA, mRNA, gRNA, miRNA, and/or DNA encoding said gene, a therapeutic ligand, or a combination thereof) for use in a method to treat brain cancer.

Also, disclosed herein are methods of treating, inhibiting, reducing, decreasing, ameliorating, and/or preventing brain cancer (such as, for example a glioblastoma), comprising implanting any of the macroporous scaffolds disclosed herein into the brain of a subject in need thereof. For example, disclosed herein are methods of treating, inhibiting, reducing, decreasing, ameliorating, and/or preventing brain cancer (such as, for example a glioblastoma), comprising implanting a macroporous scaffold comprising a viral vector (such as, for example, lentivirus, retrovirus, adenovirus, adeno-associated virus, virus-like particle, transposon, or liposome) encoding a therapeutic cargo (including, but not limited to a fusion protein, a chimeric antigen receptor (CAR) (such as, for example, a CAR T cell, CAR NK cell, CAR NK T cell, or CAR macrophage that targets CD19, CD30, CD20, Cd171, Cd80/86, c-MET, DLL-3, DR5, EpHA2, BCMA, GD2, B7H3, NKR2, NKG2D, CD133, CEA, EGFR, EGFR 806, Mesothelin, PSCA, PSMA, EpCAM, MUC1, ICAM-1, CD147, EpHA2, HER2, IL13Rα2, FOLR1, MSLN, CLDN 18.2, VEGFR2, AFP, Nectin4/FAP, Lewis Y, glypican-3, AFP, AXL, DR5, gp100, MAGE-A1/3/4, LMP1, DLL-3, IL-13 receptor α chain 2 (IL13Rα2), B7-H3, neural/glial antigen 2 (NG2), disialoganglioside GD2, epidermal growth factor receptor vIII (EGFRvIII), MUC1, PSMA, mesothelin, HER2, or CEA), an exogenous gene, siRNA, tasiRNA, lncRNA, shRNA, mRNA, gRNA, miRNA, and/or DNA encoding said gene, a therapeutic ligand, or a combination thereof) to treat brain cancer.

As would be recognized by one of ordinary skill in the art based on the present disclosure, treatment regimens can be used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Tale Powder (Talc), Steritale (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxcos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Well-connected Drydux scaffold structure supports efficient T cell reprogramming. Transduction of T cells to produce CAR T cells with stable CAR expression is a key step in CAR T cell manufacturing. Current technologies utilize transduction-promoting agents (RetroNectin, polybrene) and physical forces like centrifugation (spinoculation) to facilitate robust viral-mediated gene transfer with high transduction efficiencies. However, these multi-step techniques increase the time, cost, and complexity of the genetic modification.

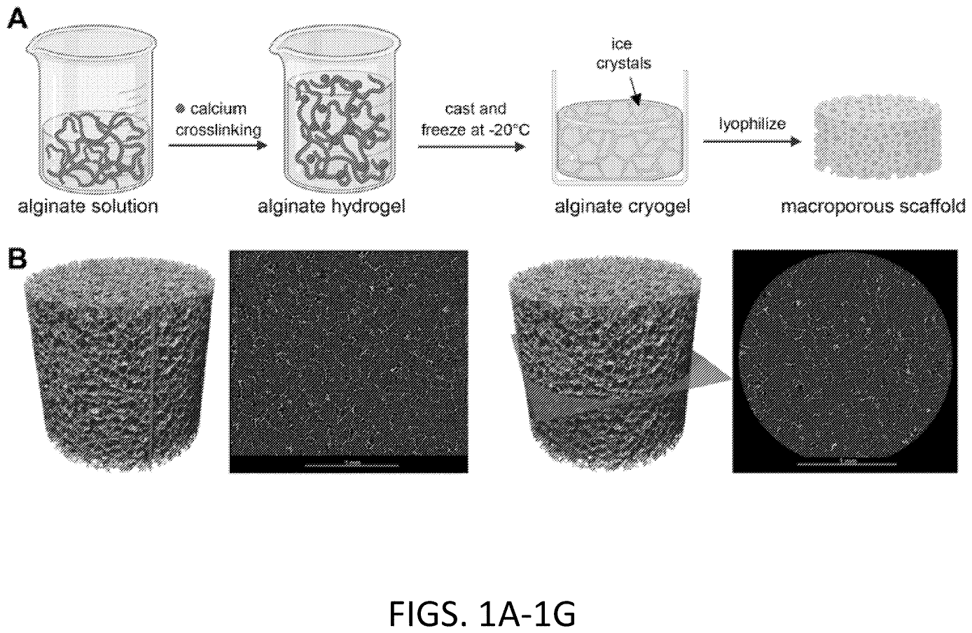

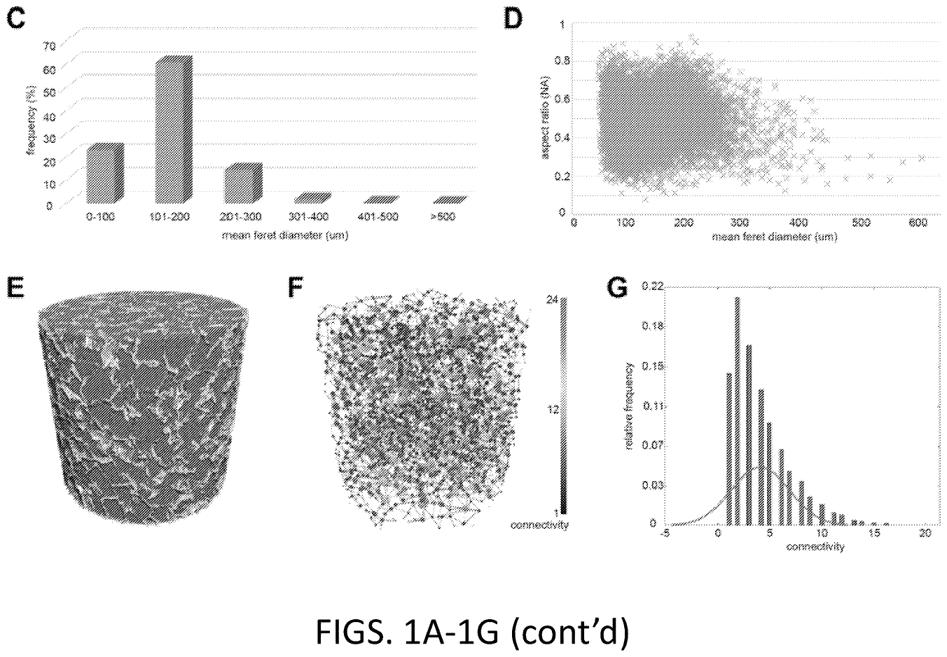

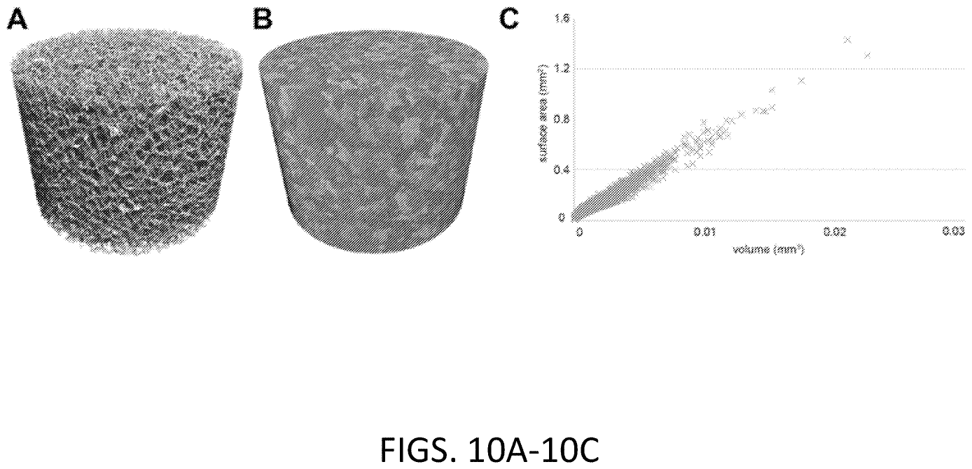

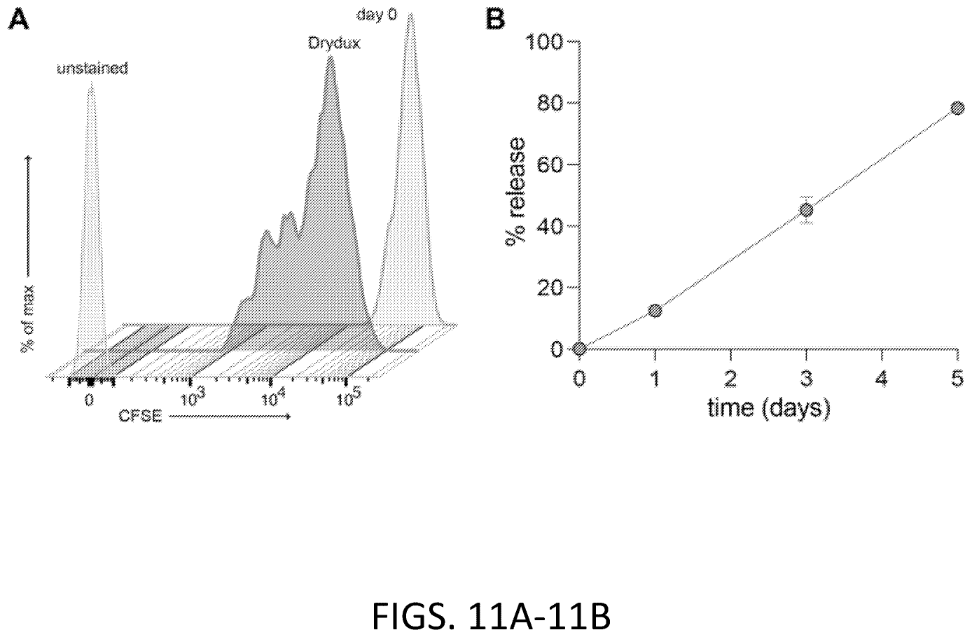

To enable single step static transduction, Drydux scaffolds were synthesized through mild cryogelation (FIG. 1A) of calcium crosslinked alginate gels. Since it has been observed that macroporosity and hygroscopy were critical for scaffold-mediated static T cell reprogramming, detailed characterization of scaffold porosity and architecture was performed. X-ray computed tomography (CT) analysis of Drydux showed 100-200 μm, oblong shaped, well-connected pores accounting for 82.78% of the scaffold volume (FIGS. 1B-1E and FIGS. 10A-10C). This well-connected macroporous structure (FIGS. 1F-1G) facilitates effective interaction between activated cells and CAR-encoding viral particles enabling gene transfer and allowing mass transfer of nutrients to support T cell proliferation (FIG. 11A) and release (FIG. 11B).

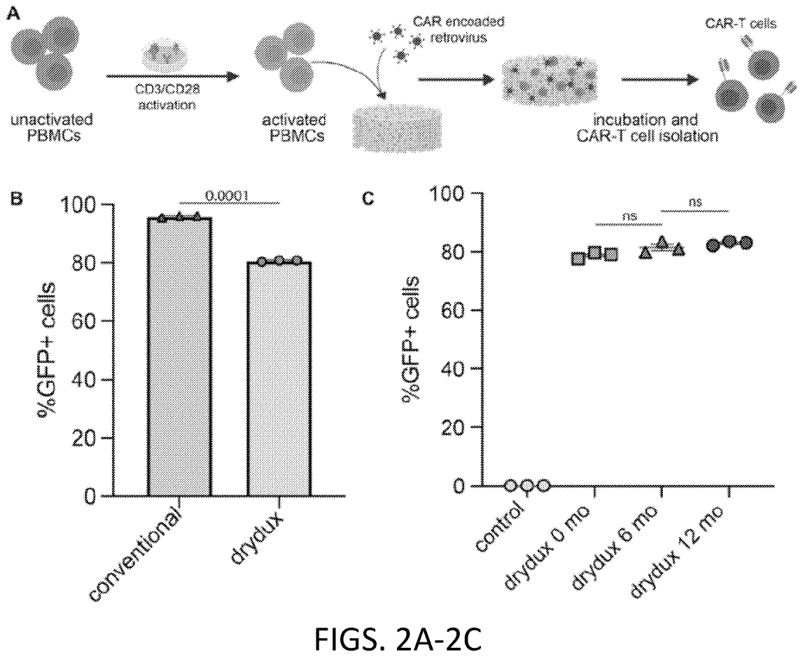

To test the potential of Drydux scaffolds to reprogram T cells in vitro, GFP-encoding retrovirus and freshly isolated and activated human peripheral blood mononuclear cells (PBMCs) were mixed and seeded onto a dry macroporous scaffolds and incubated for 3 days (FIG. 2A). Conventional CAR T cell manufacturing (spinoculation with RetroNectin-coated plates) was used as a positive control. Drydux scaffold produced ˜80% of GFP⁺ cells while conventional spinoculation produced ˜95% GFP⁺ cells (FIG. 2B). Although Drydux was marginally less efficient, both methods provided high quality transduction and the Drydux method was significantly less complex, obviating the need for sequential centrifugation.

Since RetroNectin-coated plates can be stored for up to one-two months at 4° C., the shelf-life of Drydux scaffolds was tested. Drydux scaffolds stored in a sealed bag at 4° C. for either six months, twelve months and eighteen months showed similar, if not better, transduction efficiency as compared to freshly made scaffolds, suggesting excellent shelf-life and functionality for long periods of time (FIG. 2C).

### Example 2

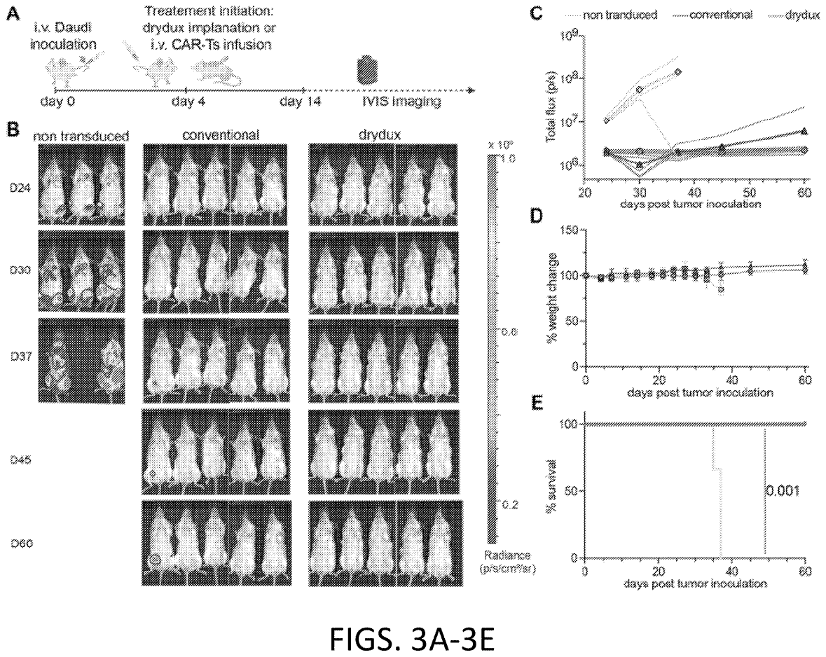

Implantable Drydux scaffolds provide excellent antitumor efficacy against lymphoma. Previous work reported that biomaterial scaffolds produce highly functional CD19.CAR T cells in vitro. Experiments were conducted to test whether this scaffold platform could generate and release functional CD19-targeting CAR T cells in vivo. The efficacy of implanted Drydux scaffolds was first explored in vivo using the well-studied Daudi lymphoma model. Drydux scaffolds were seeded with IL-2-supplemented media containing activated PBMCs, and CD19.CAR-encoding retroviral particles. Scaffolds were implanted subcutaneously in lymphoma-bearing mice 4 days after tumor cell inoculation (FIG. 3A). Tumor-bearing mice intravenously infused with conventionally generated CAR T cells served as positive controls while Drydux scaffolds seeded with activated PBMCs, but not virus, served as negative controls. CD19.CAR T cells generated in vivo using Drydux or conventional methodology, equally eradicated tumors as assessed by the measurement of tumor bioluminescence intensity (FIGS. 3B-3C). Control of the tumor was not associated with any signs of toxicity as assessed by overall appearance and measurement of body weight (FIG. 3D) as well as by improved overall survival rate (FIG. 3E). This demonstrated the potential of implantable Drydux scaffolds to generate functional CD19.CAR T cells in vivo with potency similar to conventionally generated CAR T cells but without elaborate ex vivo handling.

### Example 3

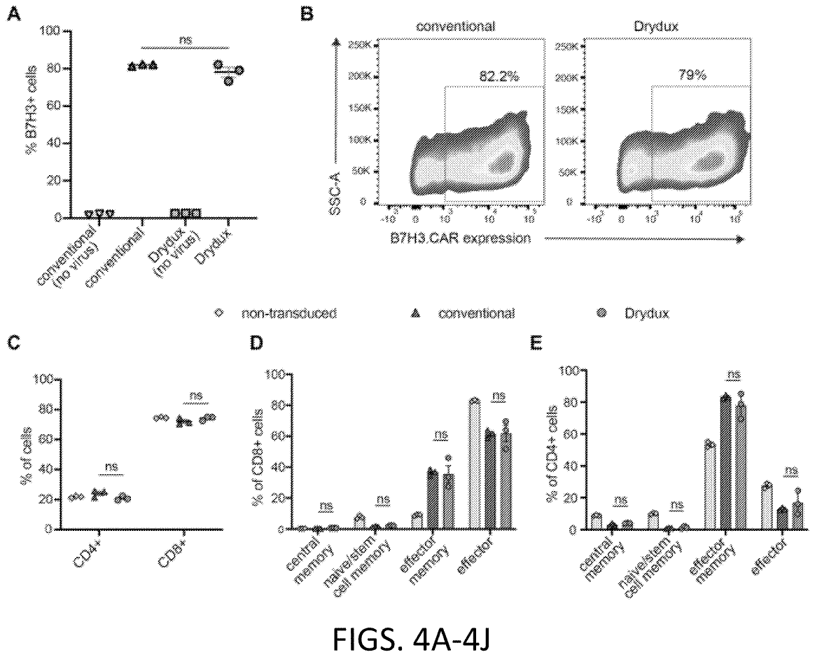

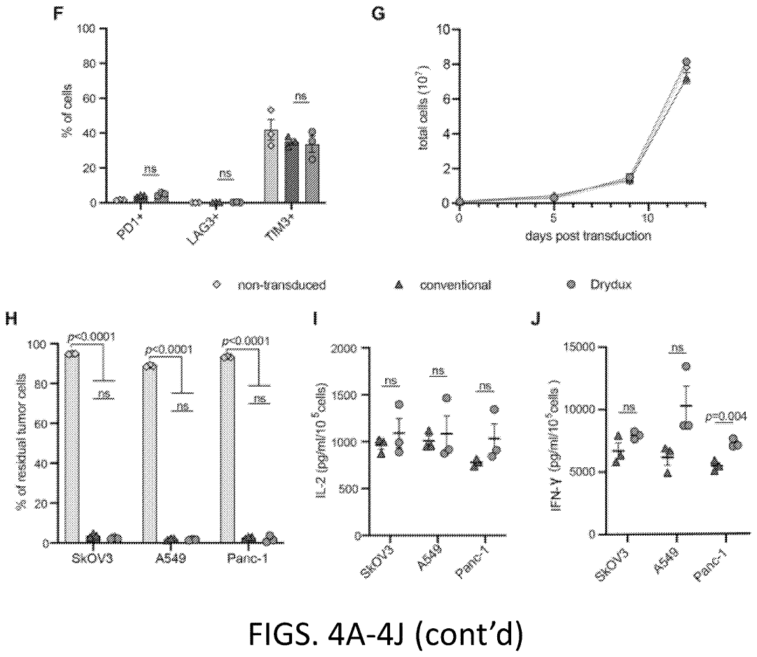

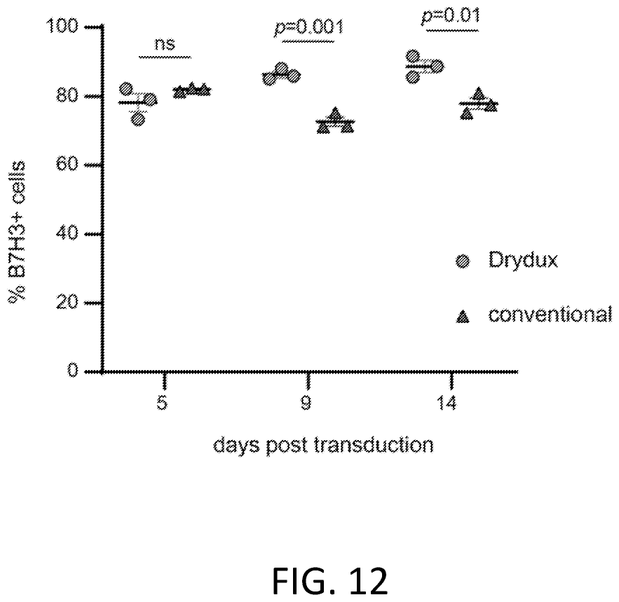

Drydux generates highly functional B7H3-targeted CAR T cells. To test the potential of Drydux scaffolds to generate CAR T cells for use against solid tumors, CAR T cells targeting the B7-H3 antigen were generated. PBMCs isolated from healthy donors were activated on αCD3/αCD28-coated plates, mixed with a gamma retrovirus encoding B7H3.CAR and seeded onto Drydux scaffolds (FIG. 2A). Cells were isolated after 2 days, and transduction efficiency was evaluated using flow cytometry. T cells reprogrammed using the conventional RetroNectin and spinoculation method were used as a positive control. Activated cells seeded onto the Drydux scaffold without gamma retrovirus served as non-transduced controls. On day 5, Drydux and conventional method showed comparable transduction efficiencies producing ˜79% and ˜82% B7H3.CAR⁺ cells, respectively (FIGS. 4A-4B). Moreover, both Drydux and conventionally generated CAR T cells had stable CAR expression over 14 days, (FIG. 12) suggesting robust, stable reprogramming. CAR T cells generated using either method showed similar percentages of CD4⁺ and CD8⁺ populations (FIG. 4C) as well as similar frequencies of naïve/stem cell memory (CD45RA⁺CCR7⁺), central memory (CD45RA⁻CCR7⁺), effector memory (CD45RA⁻CCR7⁻) and effector (CD45RA⁺CCR7⁻) population within each subset (FIGS. 4D-4E). In addition, CAR T cells generated using either method showed similar expression of inhibitory receptors or exhaustion markers like PD1, LAG3, and TIM3 (FIG. 4F). Moreover, CAR T cells generated using either method showed comparable and robust cell expansion kinetics (FIG. 4G).

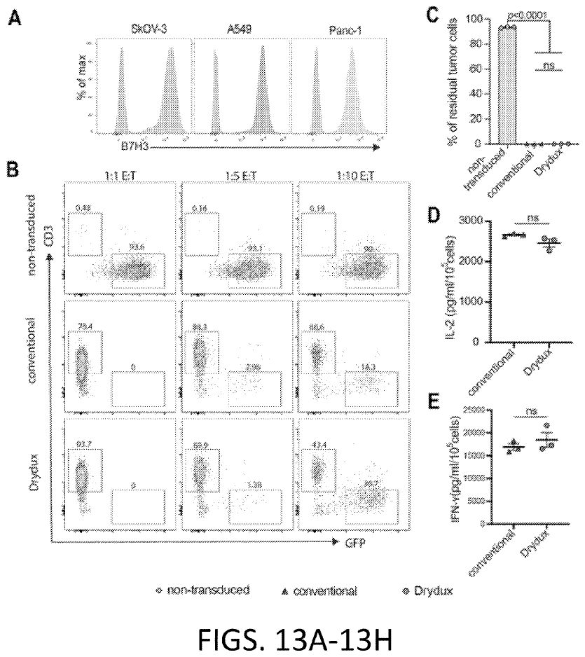

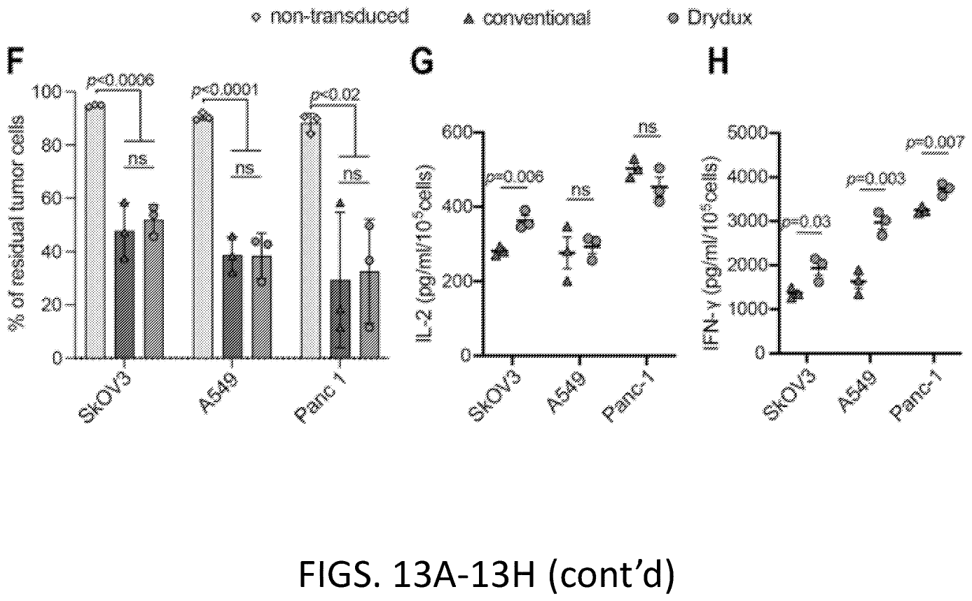

To study the functionality of B7H3.CAR T cells in vitro, Drydux and conventionally produced CAR T cells were co-cultured with B7-H3 expressing ovarian (SKOV3), lung (A549) and pancreatic cell lines (PANC-1) (FIG. 13A) at different effector to target (E: T) ratios. Non-transduced cells were used as a negative control. CAR T cells generated using conventional method or Drydux scaffold provided effective tumor growth control against ovarian, lung and pancreatic tumor cells at 1:5 E:T ratio (FIG. 4H). In addition, excellent cytotoxicity of B7H3.CAR T cells was observed at 1:1 E:T ratio against pancreatic tumor cells (FIG. 13B-13C) while partial cytolytic activity was observed against ovarian, lung and pancreatic tumor cells at 1:10 E:T ratio (FIG. 13F). Cytokine (IL-2 and IFN-γ) release by CAR T cells in response to target tumor cells was also determined (FIGS. 41-4J; FIGS. 13D-13E and FIGS. 13G-13H).

Taken together, Drydux scaffolds simplify genetic modification and generate CAR T cells with transduction efficiencies comparable to conventional methods. In addition, scaffold-generated CAR T cells expand to clinically relevant dosages, retain effector phenotype and exhibit functionality against variety of solid tumor cell lines in in vitro settings.

### Example 4

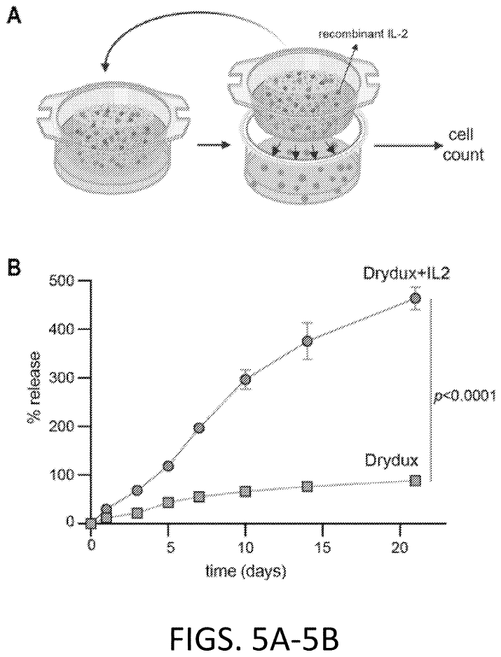

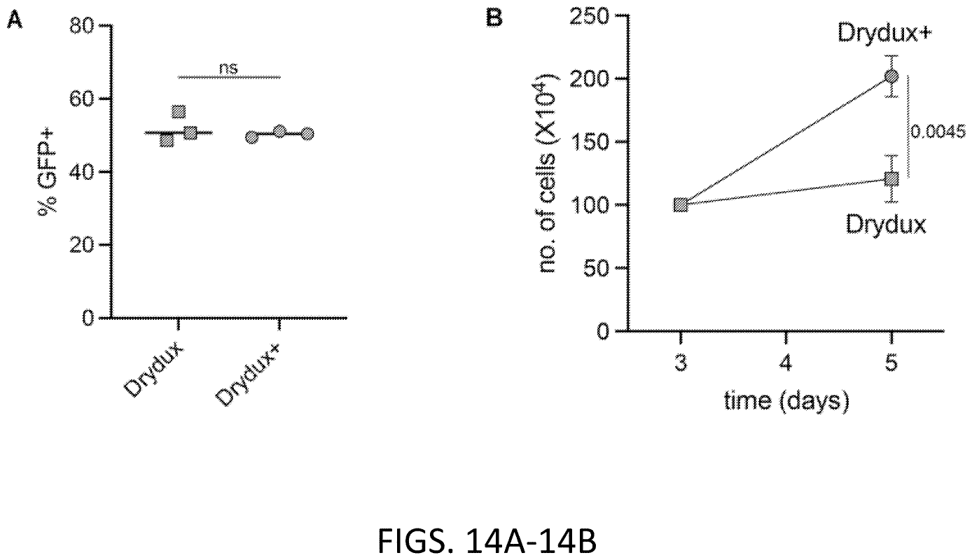

Implantable Drydux scaffold shows interleukin-mediated proliferation in vitro. To promote proliferation and release of CAR T cells following subcutaneous implantation in vivo, IL-2 was physically encapsulated in the Drydux (Drydux+IL2) scaffold. The transduction efficiencies of Drydux and Drydux+IL2 were comparable (FIG. 14A), suggesting IL-2 does not influence transduction. Further, Drydux+IL2 scaffolds also supported T cell proliferation without the need to add additional cytokines to media (FIG. 14B). Moreover, IL-2 significantly facilitated cell release from the scaffold as measured by in vitro cell release assay. Drydux+IL2 scaffolds placed in transwell inserts released cells into the bottom chamber over 21 days (FIGS. 5A-5B), demonstrating the potential of Drydux+IL2 scaffold to facilitate sustainable release of CAR T cells following in vivo implantation.

### Example 5

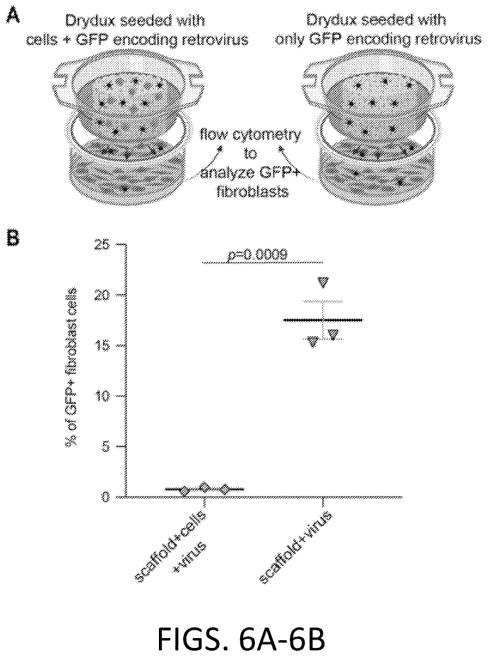

Implantable Drydux scaffold demonstrates absence of inadvertent transduction in vitro. Finally, experiments were conducted to test the possibility of retroviral leakage and surrounding host cells transduction upon implantation of scaffolds loaded with activated PBMCs and CAR-encoding retrovirus. An in vitro transwell experiment was designed wherein Drydux loaded with PBMCs and GFP-encoding retrovirus was placed in a transwell insert and human fibroblast cells were seeded in the bottom chamber. At a determined time point, GFP expression in the fibroblast was assessed to identify retroviral leakage and undesired transduction (FIG. 6A). When Drydux was seeded with GFP-encoding retrovirus alone, ˜17% GFP expression was observed in the fibroblasts, indicating virus leakage from the scaffold in the absence of co-seeded PBMCs. In contrast, when Drydux scaffolds were seeded with both PBMCs and GFP-encoding retrovirus, no transduction was observed in the fibroblast cells, suggesting virus encounters and transduces the cells at its closest proximity (FIG. 6B). This serves as an indirect demonstration suggesting Drydux scaffold will likely not transduce host cells upon implantation in the subcutaneous space.

### Example 6

Implantable Drydux scaffolds outperform conventionally generated CAR T cells against metastatic lung tumors. Motivated by the ability of the Drydux scaffolds to generate and release tumor-specific CAR T cells in vitro, experiments were conducted to test the potential of this platform in solid tumors. It was hypothesized that in vivo generated CAR T cells would not only reduce the manufacturing time and costs but also generate less differentiated, long-lasting, and highly functional CAR T cells against hard-to-treat solid tumors. Therefore, the potential of Drydux+IL2 scaffolds to generate B7-H3 targeting CAR T cells in vivo was tested against a panel of solid tumor models.

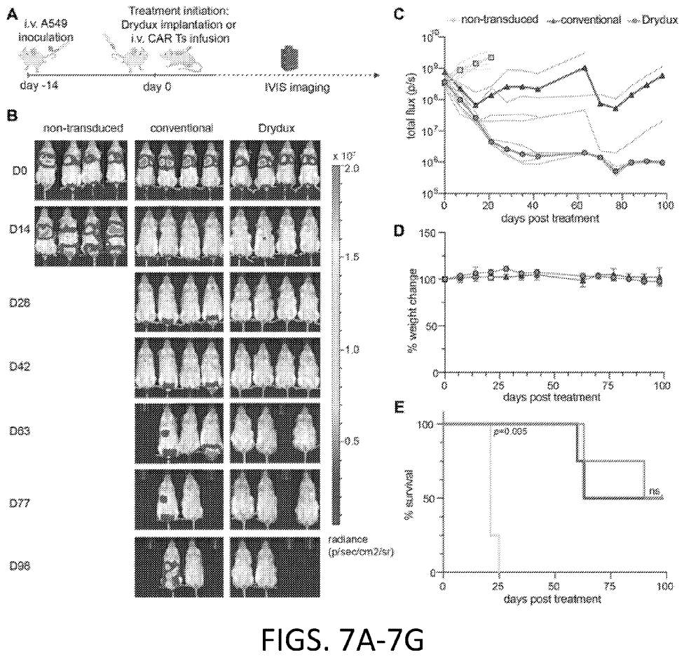

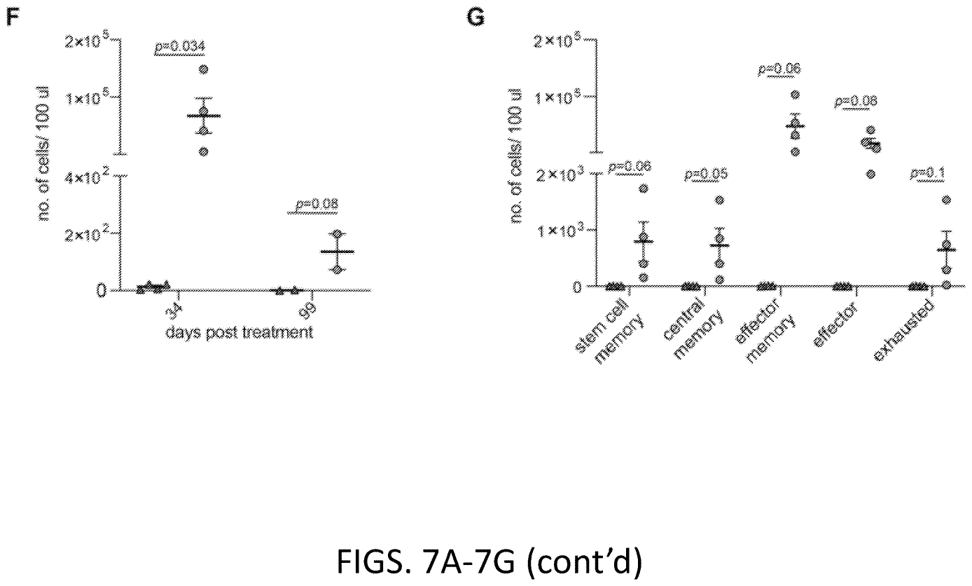

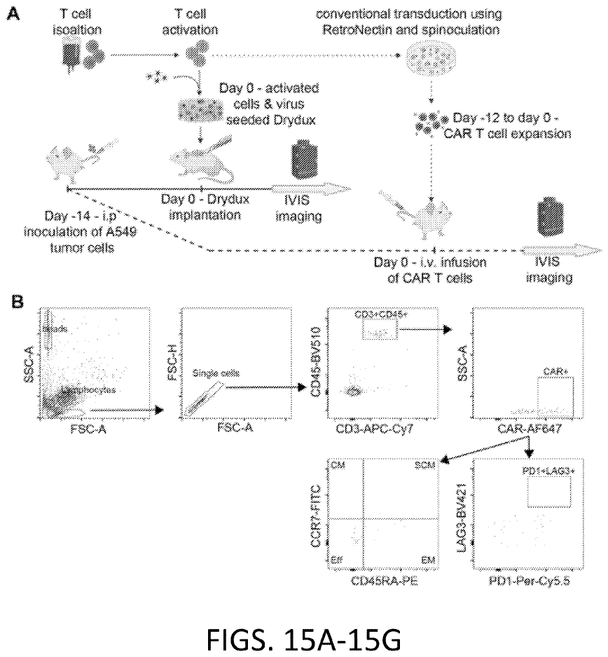

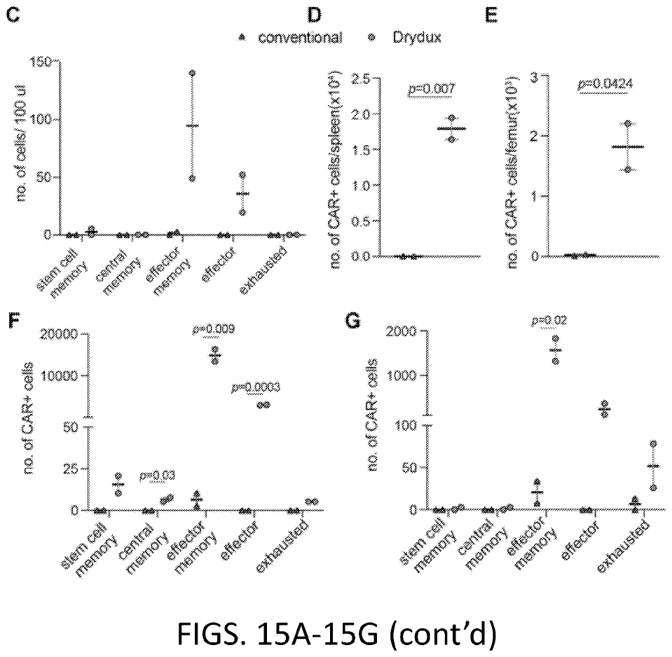

First, the antitumor effects of Drydux generated CAR T cells were evaluated in a metastatic model of non-small cell (NSC) lung tumors. Tumors were established by inoculating FFluc-expressing A549 cells infused via tail vein injection in NSG mice. Treatment was initiated 14 days after tumor cell inoculation. Each animal received donor-matched non-transduced T cells seeded Drydux+IL2 scaffolds or CAR T cells generated using a conventional method or Drydux+IL2 scaffolds seeded with activated cells and CAR-encoding retrovirus (CAR T cell dose ˜2.5×10⁶) (FIG. 7A, FIG. 15A). While the tumors grew rapidly in the control animals receiving non-transduced T cells, animals receiving conventionally generated CAR T cells or Drydux+IL2 scaffolds effectively controlled tumor growth up to day 14 post treatment. However, after 14 days, animals receiving Drydux+IL2 scaffolds showed continued tumor regression while animals receiving conventionally produced CAR T cells showed signs of tumor relapse. Moreover, CAR T cells generated in vivo using Drydux+IL2 scaffolds provided long-lasting antitumor effects (FIGS. 7B-7C). Neither of the two groups receiving CAR T cells showed weight loss, suggesting this therapy was well tolerated for treatment duration (FIG. 7D), excepting the late-stage formation of GVHD, which is a ubiquitous complication of using human cells in a mouse model. Although both the treatments showed similar improvements in survival, surviving animals treated with Drydux+IL2 remained tumor free up to day 98 post treatment (FIG. 7E).

To assess the ability of CAR T cells to persist in vivo following treatment, analysis of CAR+ cells was performed using flow cytometry at predetermined time points. Assessment of the number and immunophenotypic composition of circulating CAR T cells 34 days post treatment revealed a higher number of circulating CAR⁺ cells in the group receiving Drydux+IL2 scaffolds (FIG. 7F). CAR⁺ population showed predominantly effector memory and effector phenotype along with a few circulating stem cell memory, central memory, and exhausted (PD1⁺LAG3⁺) cells (FIG. 7G) suggesting robust in vivo expansion of CAR T cells while maintaining highly functional, less differentiated phenotype. At the end of the experiment (day 99), blood, spleen, and bone marrow were collected from the surviving animals, and CAR T cell number and their immunophenotypic composition was assessed. At day 99, animals treated with Drydux+IL2 scaffolds showed circulating CAR T cells with predominately effector memory and effector phenotype (FIG. 15C). CAR T cell analysis from the spleen (FIG. 15D) and bone marrow (femur) (FIG. 15E) showed a significant presence of effector memory and effector CAR T cells (FIGS. 15F-15G) suggesting long lasting antitumor capacity of Drydux+IL2 scaffold-generated CAR T cells.

Taken together, CAR T cells generated in vivo on Drydux+IL2 scaffolds demonstrated higher functionality and improved persistence resulting in prolonged antitumor effects and prevention of tumor recurrence in the metastatic lung tumor model.

### Example 7

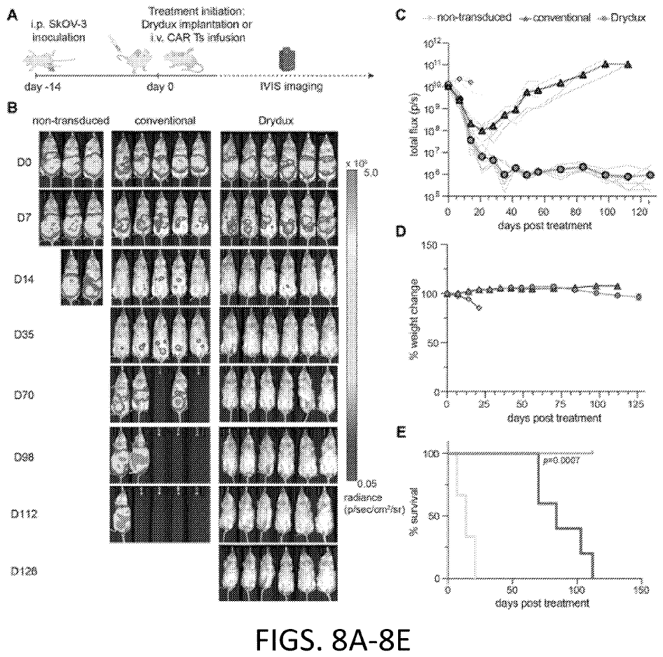

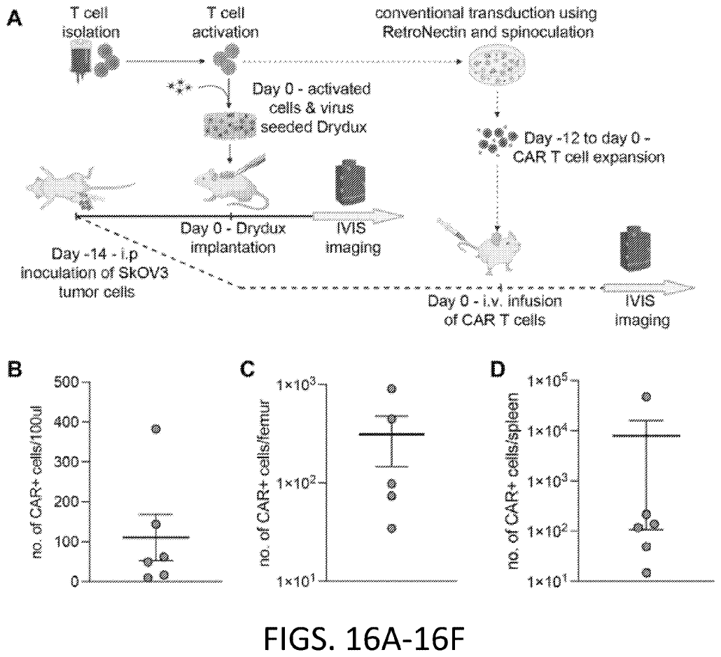

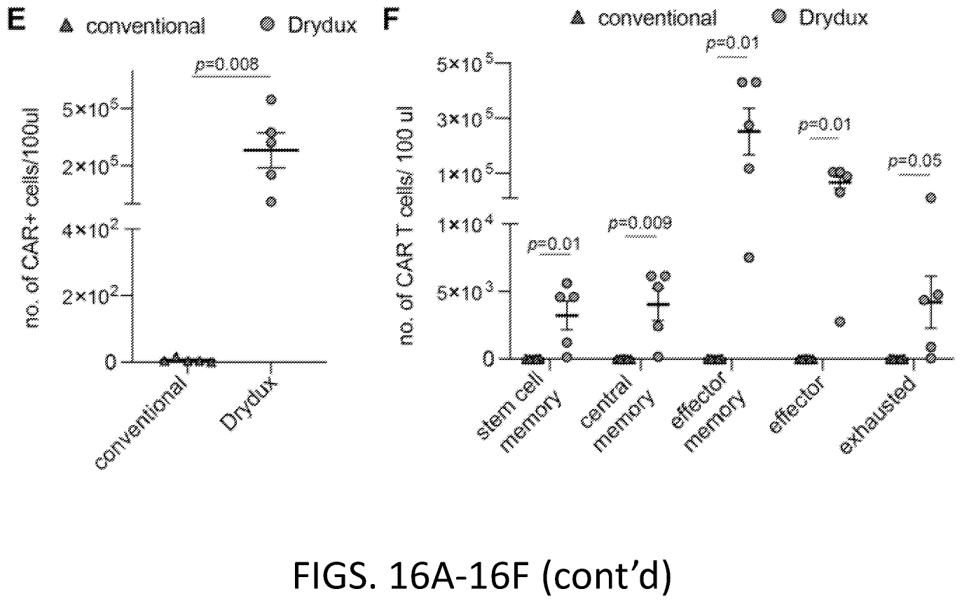

Implantable Drydux scaffolds generate highly functional CAR T cells against ovarian tumors. Following the excellent antitumor effects of Drydux generated CAR T cells in metastatic lung tumors, the potential of this platform was evaluated in an intraperitoneal ovarian xenograft model. FFluc-expressing SKOV3 tumor cells were inoculated intraperitoneally in NSG mice and treatment was initiated on day 14. Each animal received donor-matched 2.5×10⁶ CAR T cells generated using a conventional method or Drydux+IL2 scaffolds seeded with cells and CAR-encoding retrovirus. Negative control animals received Drydux+IL2 scaffolds with PBMCs only (FIG. 8A, FIG. 16A). Tumor growth was monitored until day 126 post treatment. Animals receiving control scaffolds demonstrated rapid tumor progression and, interestingly, GVHD progression within 15 days of treatment. Both conventionally and Drydux+IL2 scaffold generated B7H3.CAR T cells demonstrated significant tumor eradication but conventionally treated animals showed signs of tumor relapse within 30 days post treatment. On the other hand, animals receiving Drydux+IL2 scaffolds showed prolonged antitumor effects, and all treated mice remained tumor-free up through the end of the experiment (126 days post-treatment) (FIGS. 8A-8C). Neither group demonstrated significant weight loss, suggesting they tolerated the CAR T cell dosage well (FIG. 8D-8E). Additionally, animals receiving Drydux+IL2 scaffold generated CAR T cells showed significant improvement in overall survival compared to conventionally treated animals (FIG. 8E).

To evaluate CAR T cell persistence in blood, CAR T cells from blood, bone marrow and spleen of the surviving animals were collected. At day 123, animals treated with scaffold generated CAR T cells showed circulating B7H3.CAR⁺ cells highlighting the ability of in vivo generated CAR T cells to persist longer in circulation even after tumor remission (FIG. 16B). CAR T cell analysis from the bone marrow (femur) and spleen showed a significant presence B7H3.CAR T cells (FIGS. 16C-16D). No animals treated with conventionally generated CAR T cells were living at the final data point and so could not be analyzed for CAR T cells. To quantify CAR T cells in blood at earlier time points, this tumor model was repeated with a second donor and animals receiving CAR T cells generated from a second donor were evaluated at day 34 post treatment. At day 34, Drydux+IL2 scaffold-treated mice had considerably higher numbers of circulating CAR T cells as compared to animals treated with the same dose of conventionally produced CAR T cells (FIG. 16E). The immunophenotypic assessment showed predominantly effector memory and effector phenotype. In addition, mice treated with Drydux+IL2 generated CAR T cells showed higher circulating numbers of less differentiated stem cell memory as well as central memory cells (FIG. 16F). Taken together, Drydux generated CAR T cells showed superior antitumor effects and prevented tumor relapse compared to conventionally generated CAR T cells likely due to improved persistence in vivo.

### Example 8

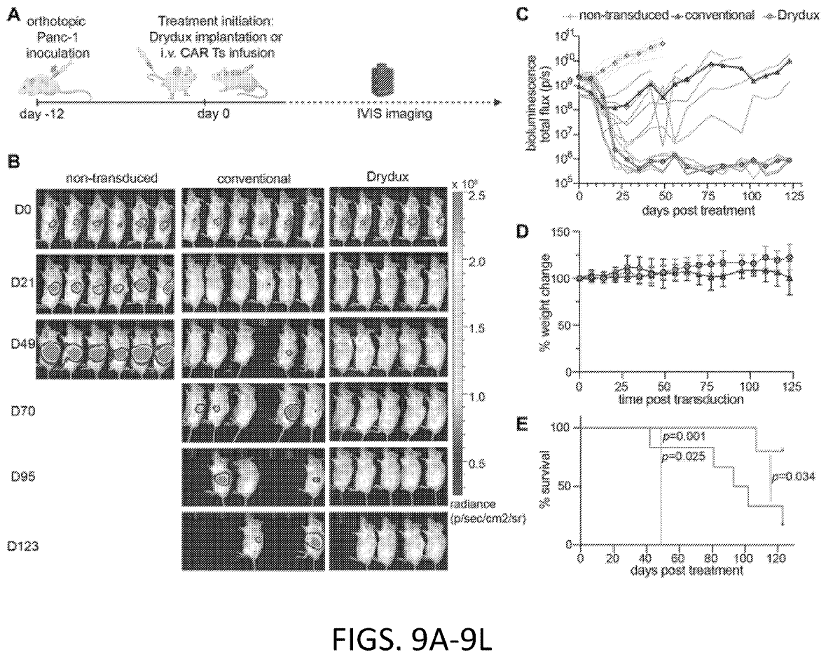

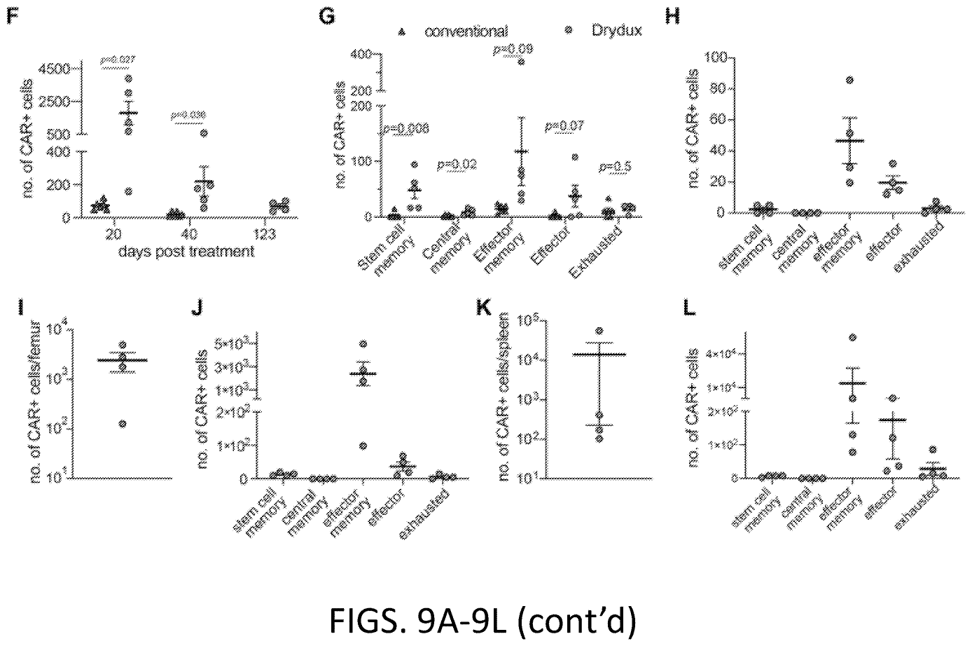

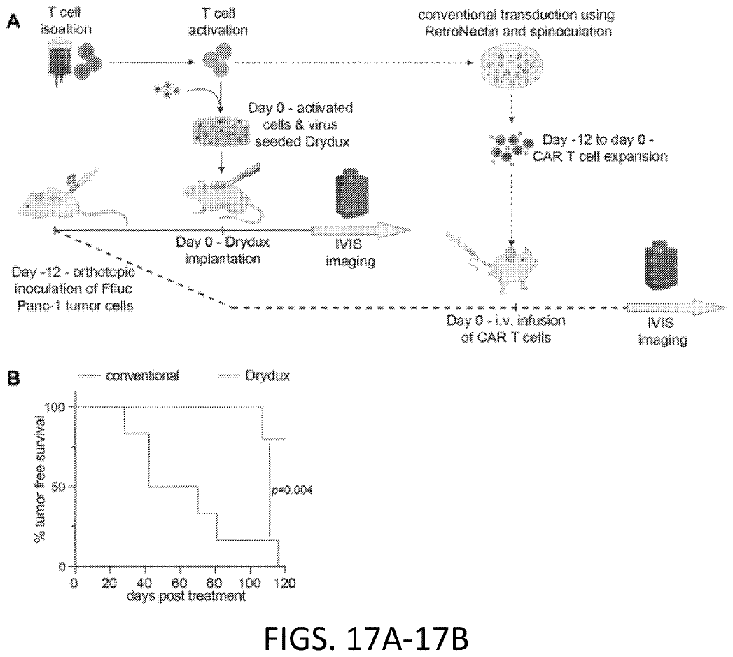

Implantable Drydux scaffolds generate highly functional and persistent CAR T cells and prevent tumor relapse in orthotopic pancreatic tumors. Finally, the Drydux platform was tested for the ability to generate functional CAR T cells against an aggressive and lethal orthotopic pancreatic tumor model. FFluc-expressing Panc-1 cells were inoculated into the pancreas of NSG mice and treatment was initiated after 12 days. Each animal received donor-matched 2.5×10⁶ non-transduced T cells seeded Drydux+IL2 scaffolds or CAR T cells generated using conventional method or Drydux+IL2 scaffolds seeded with cells and CAR-encoding retrovirus (CAR T cell dose ˜2.5×10⁶) (FIG. 9A, FIG. 17A). Negative control animals receiving Drydux+IL2 and PBMCs, but no virus demonstrated rapid tumor growth. Animals receiving i.v. infusion of conventionally generated CAR T cells demonstrated excellent initial tumor regression but succumbed to gradual tumor recurrence in days 70-100 following treatment. In sharp contrast, animals receiving Drydux+IL2 scaffolds showed prolonged antitumor effects, and all treated mice remained tumor-free up through the end of the experiment (123 days post-treatment) (FIGS. 9B-9C). Animal weight monitoring suggested that the treatment was well tolerated throughout the entire duration and no apparent toxicity associated with CAR T cell therapy was seen (FIG. 9D). Moreover, animals receiving Drydux+IL2 scaffold generated CAR T cells showed significant improvement in overall (FIG. 9E), as well as tumor-free survival (FIG. 17B), compared to animals treated with the same dose of conventionally generated CAR T cells.

To evaluate the in vivo persistence of CAR T cells, blood was collected at day 20 and day 40 post treatment. Animals receiving Drydux+IL2 scaffolds had almost 24-fold higher B7H3.CAR⁺ cells at day 20 and 10-fold higher B7H3.CAR⁺ cells at day 40 compared to conventionally generated CAR T cells (FIG. 9F). Additionally, immunophenotypic analysis was performed on CAR T cells in the blood at day 40 to evaluate the differentiation state of circulating CAR T cells post treatment. While animals treated with Drydux+IL2 scaffolds demonstrated higher numbers of naïve/stem cell memory, effector memory, and effector population, conventionally generated CAR T cells only showed a few effector memory cells, suggesting superior persistence and antitumor potential of Drydux generated CAR T cells. Additionally, both groups showed a similar number of exhausted cells (FIG. 9G). At the end of the experiment (day 123), the blood, spleen, and bone marrow of surviving animals were collected, and CAR T cells were quantified and submitted to immunophenotypic analysis. At day 123, animals treated with scaffold generated CAR T cells showed circulating B7H3.CAR⁺ cells with predominately effector memory and effector phenotypic composition with few circulating naïve/stem cell memory cells and some exhausted population (FIG. 9H) further highlighting the ability of in vivo generated CAR T cells to persist longer in circulation even after tumor remission. CAR T cell analysis from the spleen and bone marrow (femur) showed a significant presence of effector memory followed by few effector CAR T cells (FIGS. 91-9L). No animals treated with conventionally generated CAR T cells were tumor-free at the final data point and so could not be analyzed for CAR T cells.

Taken together, CAR T cells generated in vivo using the Drydux platform were highly functional against an orthotopic pancreatic tumor. Additionally, implantable macroporous scaffolds provided sustained cellular delivery of less differentiated CAR T cells and showed improved persistence in vivo preventing disease relapse. Moreover, the Drydux scaffold was implanted within three days of T cell isolations, reducing time and complexity associated with CAR T cell manufacturing.

In these examples, the results demonstrate simple, stable, implantable ‘Drydux’ scaffold to generate highly functional CAR T cells at accelerated time frames. Drydux scaffolds provide favorable architecture for effective T cell reprogramming and release. This scaffold acts as an excellent transduction reagent to generate highly functional CAR T cells. Furthermore, this platform can be easily tuned for in vivo applications and require minimal ex vivo manipulation reducing the complexity of current CAR T cell manufacturing protocols. Upon subcutaneous implantation, the Drydux scaffold generated highly functional CAR T cells in animal models of systemic lymphoma, intraperitoneal metastatic ovarian, intravascular metastatic lung, and orthotopic pancreatic cancer. Additionally, the Drydux scaffold considerably improved the CAR T cell persistence, with circulating cells detectable up to 120 days after treatment, providing enhanced efficacy compared to equal numbers of conventionally generated CAR T cells. This tunable platform has the potential to improve the scope and access to costly and time-consuming cellular therapies.

### Example 9

Developing the next generation of cellular therapies will depend on fast, versatile, and efficient cellular reprogramming. Novel biomaterials will play a central role in this process by providing scaffolding and bioactive signals that shape cell fate and function. Previous investigations reported that dry macroporous alginate scaffolds mediate retroviral transduction of primary T cells with efficiencies that rival the gold-standard clinical spinoculation procedures, which involve centrifugation on Retronectin-coated plates. This scaffold transduction required the scaffolds to be both macroporous and dry. Transduction by dry, macroporous scaffolds, termed “Drydux transduction,” provides a fast and inexpensive method for transducing cells for cellular therapy, including for the production of CAR T cells.

In these examples, the mechanism of action by which Drydux transduction works was investigated through exploring the impact of pore size, stiffness, viral concentration, and absorption speed on transduction efficiency. Results demonstrated that Drydux scaffolds with macropores ranging from 50-230 μm and with Young's moduli ranging from 25-620 kPa all effectively transduce primary T cells, suggesting that these parameters are not central to the mechanism of action, but also demonstrating that Drydux scaffolds can be tuned without losing functionality. Increasing viral concentrations led to significantly higher transduction efficiencies, demonstrating that increased cell-virus interaction is necessary for optimal transduction. Finally, it was discovered that the rate with which the cell-virus solution is absorbed into the scaffold is closely correlated to viral transduction efficiency, with faster absorption producing significantly higher transduction. A computational model of liquid flow through porous media validates this finding by showing that increased fluid flow substantially increases collisions between virus particles and cells in a porous scaffold. Taken together, these data demonstrate that the rate of liquid flow through the scaffolds, rather than pore size or stiffness, serves as a central regulator for efficient Drydux transduction.

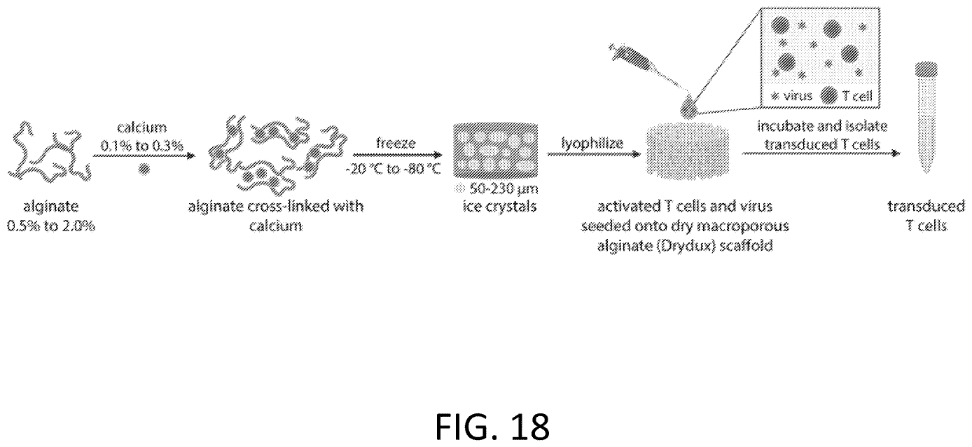

Scaffold fabrication. Macroporous scaffolds were fabricated through cryogelation (FIG. 18) Briefly, an equal volume of calcium and alginate solutions were vigorously mixed and cast into wells of a 24-well plate. Samples were then frozen and lyophilized to create dry macroporous alginate scaffolds, which were referred to as “Drydux” scaffolds.

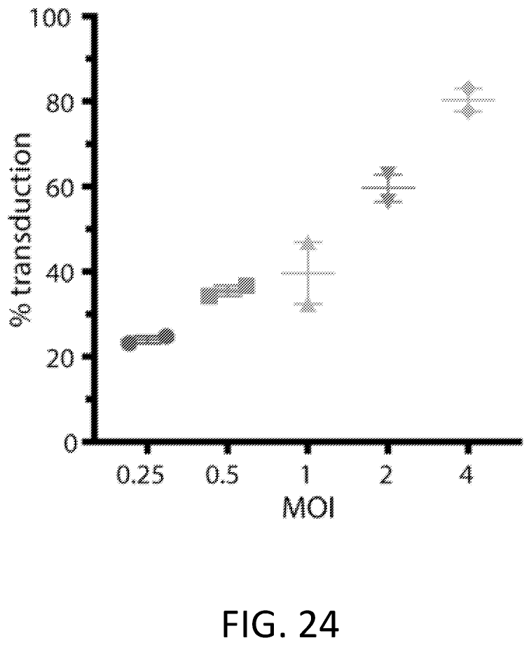

MOI calibration. Previous publications reported conditions for excellent transduction efficiencies of 85-95%. However, it was potentially problematic that these high efficiencies could hide small improvements during scaffold optimization. Therefore, the multiplicity of infection (MOI) of GFP-encoding gamma-retrovirus was titrated to achieve a transduction efficiency of below 60% against primary PBMCs isolated from human blood, reasoning that incremental improvements would be observed more easily by doing so. Lowering the MOI led to a reduction of transduction percent (FIG. 24). An MOI of 2, producing 59% transduction efficiency, was determined to be optimal and used for all following experiments, unless indicated otherwise.

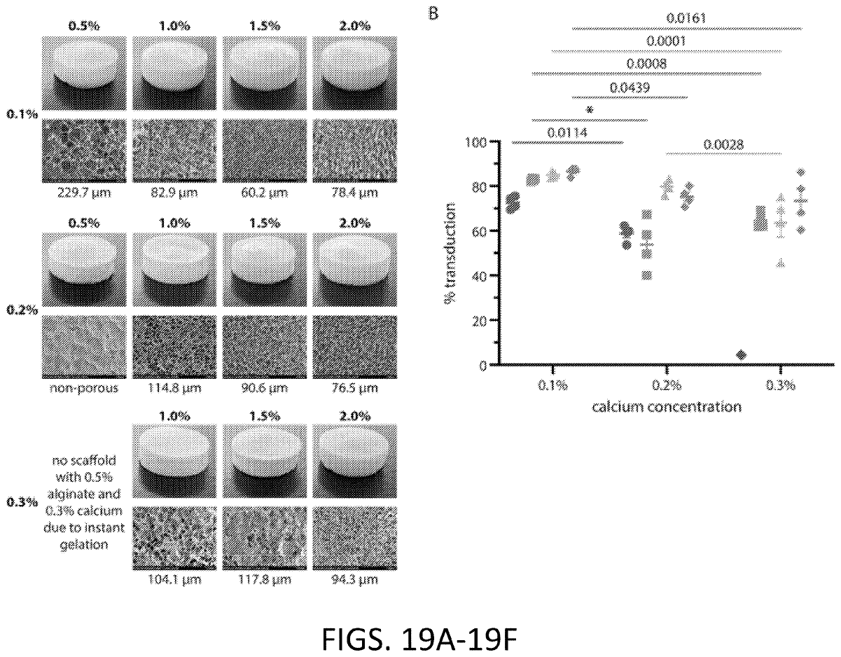

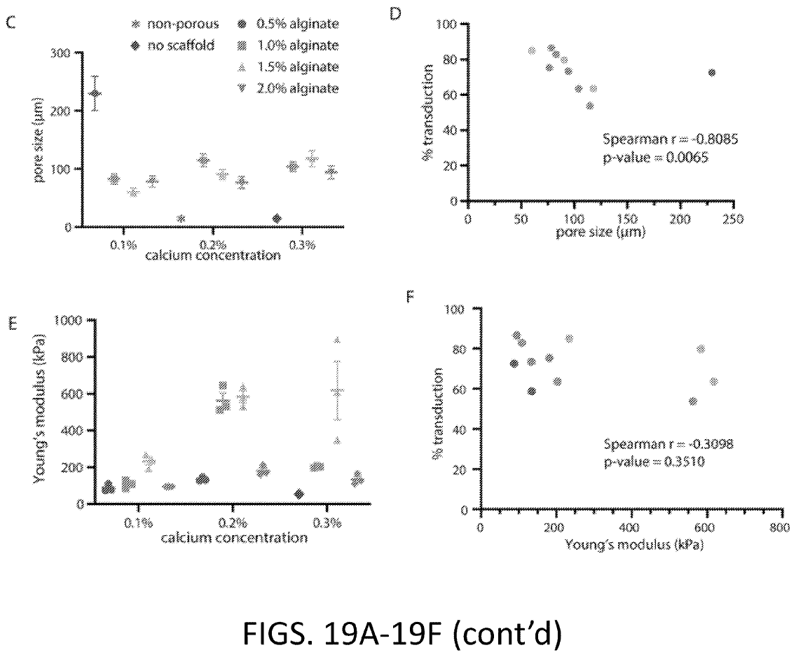

Pore size, but not stiffness, is correlated with Drydux transduction efficiency when varying alginate and calcium concentrations. To assess whether calcium or alginate concentration impacted Drydux transduction, scaffolds were formulated with varying calcium (0.1%, 0.2%, 0.3%) and alginate (0.5%, 1.0%, 1.5%, 2.0%) concentrations (w/v). The scaffolds had a cross-sectional area of ˜1.72 cm² and a height of ˜5.37 mm (FIG. 19A). All the scaffolds produced transduction efficiencies above 50%, indicating the scaffolds were highly capable of transducing cells (FIG. 19B). Scaffolds made with 0.1% calcium had significantly higher transduction efficiencies than those made with 0.2% and 0.3% calcium. Scaffolds made with 0.5% alginate showed significantly lower transduction than almost all other alginate concentrations, likely due to the lack of surface porosity of the 0.5% alginate scaffolds compared to the other scaffolds. The average pore size of these scaffolds ranged from 76-230 μm (FIG. 19C). and fitting a Spearman correlation indicated there was a strong and significant (p=0.0065) correlation between pore size and transduction efficiency (FIG. 19D).

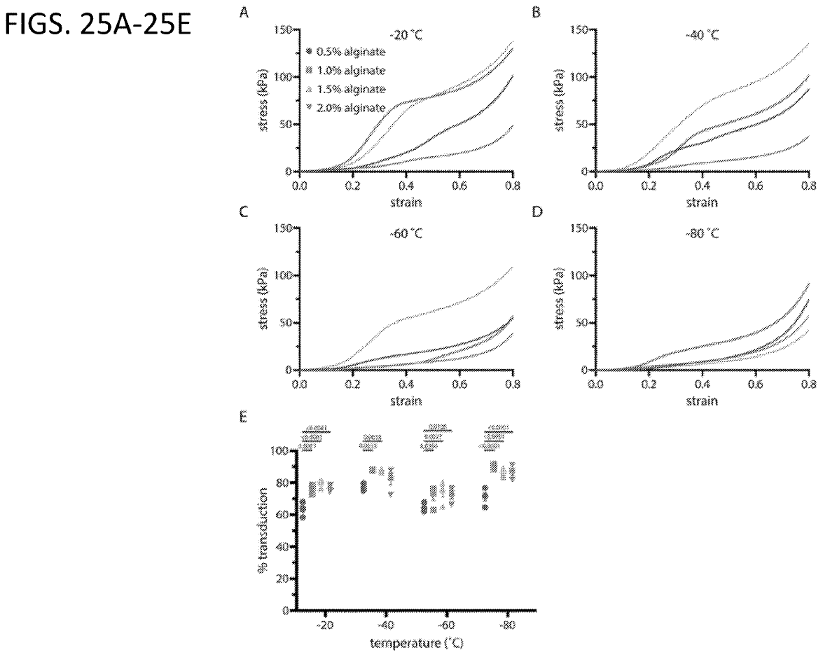

To determine scaffold stiffness, Drydux scaffolds were submitted to compression testing with a 50 N force and compression rate of 0.1 mm/s. The Young's moduli were calculated based on stress-strain curves generated from compression testing (FIG. 25). Scaffolds with 0.1% calcium (w/v) were softer than scaffolds with 0.2% and 0.3% calcium (w/v), and an alginate concentration of 1.5% (w/v) formed the stiffest scaffolds (FIG. 19E). There was not a significant (p=0.3510) Spearman correlation between the Young's modulus of the scaffold and transduction efficiency (FIG. 19F).

### Example 10

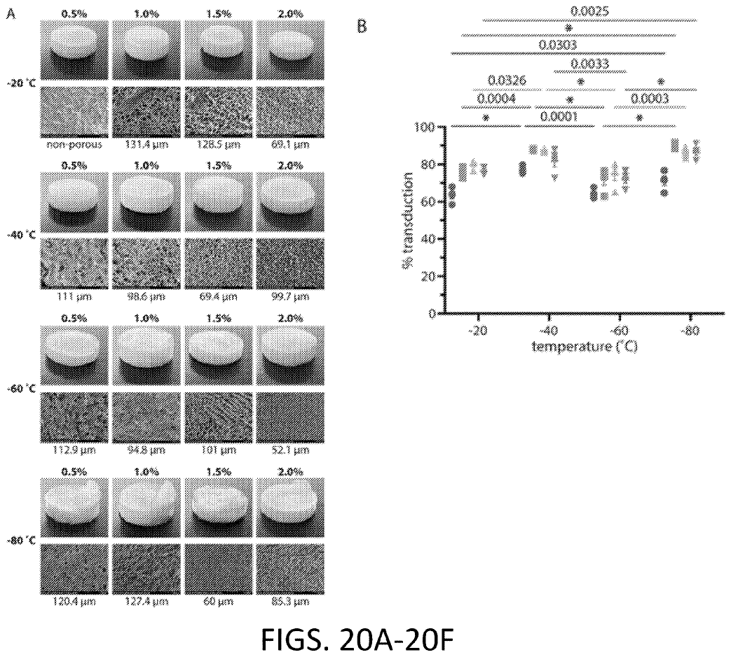

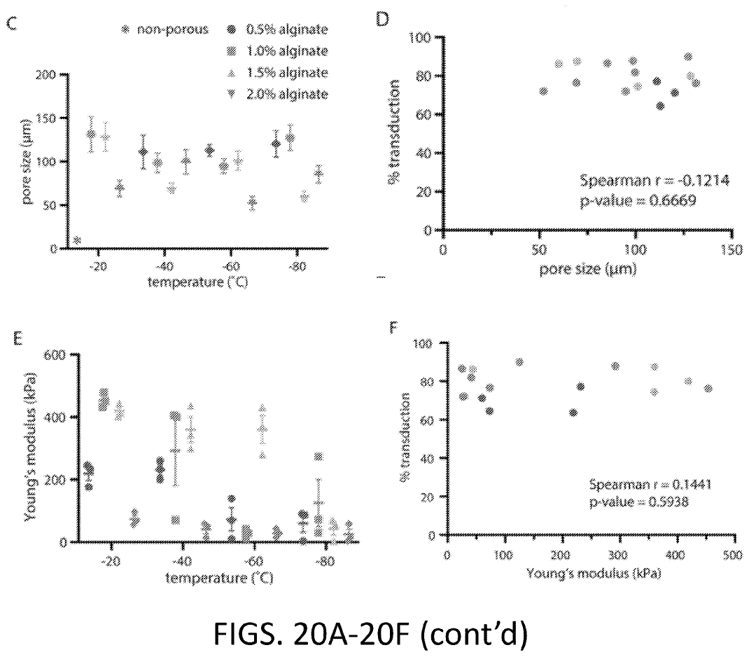

Neither pore size nor stiffness is correlated with Drydux transduction efficiency when varying alginate concentrations and freezing temperature. Several groups have reported that the freezing temperature during cryogelation determines pore size of cryogels. To further evaluate the impact of pore size, without the complication of changing crosslinked concentration, how changing the freezing temperature impacts scaffold pore size and stiffness was evaluated, and whether these changes impact transduction efficiency was further evaluated. Scaffolds were again synthesized using cryogelation with varying alginate concentrations (0.5%, 1.0%, 1.5%, 2.0%), but with a constant calcium concentration of 0.2%, in line with previous reports. Scaffolds were then frozen at −20° C., −40° C., −60° C., or −80° C. and lyophilized. The scaffolds had a cross-sectional area of ˜1.78 cm² and a height of ˜4.86 mm (FIG. 20A). All the scaffolds showed transduction efficiencies above 60%, indicating all the scaffolds successfully transduced cells (FIG. 20B). As observed above, scaffolds with 0.5% alginate showed significantly worse transduction efficiency than scaffolds made at the other alginate concentrations. There was no significant difference between the 1.0%, 1.5%, and 2.0% alginate concentration scaffolds. Scaffolds frozen at −40° C. and −80° C. displayed no significant difference in transduction, but both demonstrated significantly higher transduction than scaffolds frozen at −20° C. and −60° C. The average pore size of these scaffolds ranged from 52-131 μm (FIG. 20C). In contrast to the results shown in FIG. 19. fitting a Spearman correlation did not produce any significant (p=0.6669) correlation between pore size and transduction efficiency in this experiment (FIG. 20D).

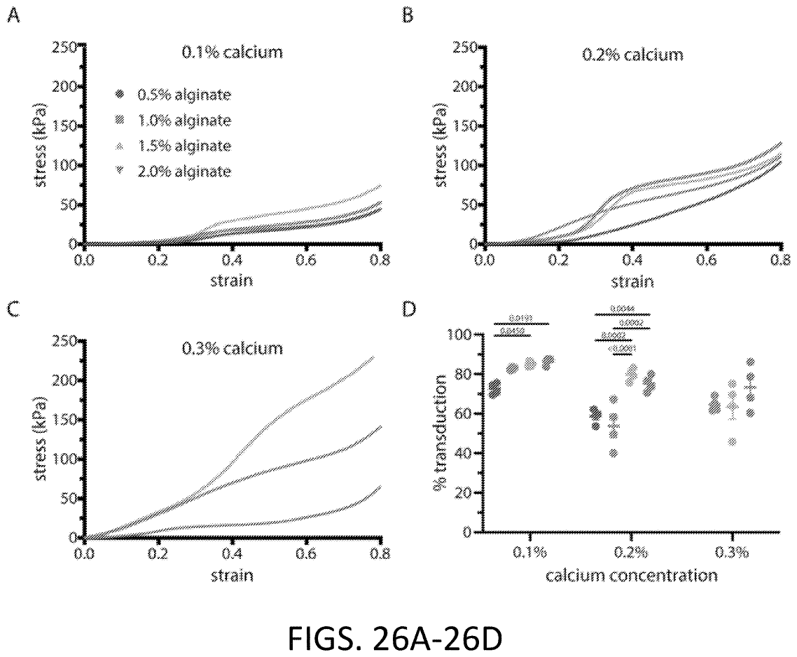

Drydux scaffolds were compressed with a 50 N force and compression rate of 0.1 mm/s to determine scaffold stiffness. The Young's moduli were calculated based on stress-strain curves generated from compression testing (FIG. 26). Slower freezing rates generally led to stiffer scaffolds, and scaffolds with 1.0% and 1.5% alginate concentrations formed the stiffest scaffolds (FIG. 20E). There was no significant (p=0.5938) Spearman correlation between the Young's modulus of the scaffold and transduction efficiency (FIG. 20F).

### Example 11

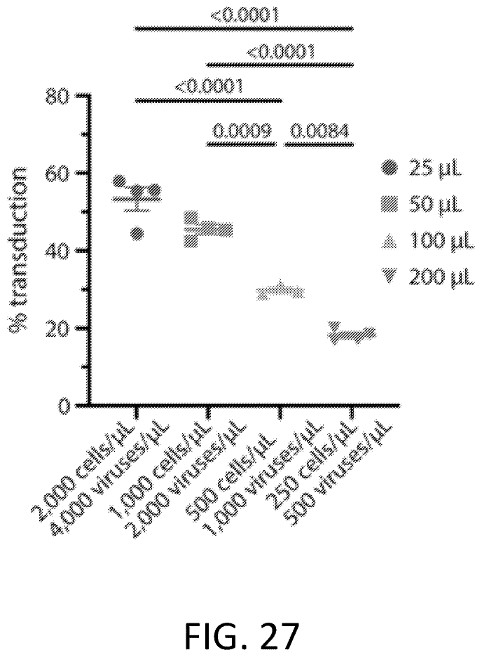

Both viral concentration and seed volume significantly correlate to Drydux transduction efficiencies. Since Drydux transduction did not appear to depend on stiffness and had an unpredictable relationship with pore size, experiments were conducted to identify other factors that might influence transduction efficiency. Viral transduction relies on interactions between virus and cells. It was reasoned that higher viral concentrations should lead to higher transduction efficiency. About 50,000 primary human PBMCs and 100,000 gamma retrovirus particles were suspended in 25 μL, 50 μL, 100 μL, or 200 μL and evaluated Drydux transduction of these solutions. Diluting the virus significantly reduced transduction, confirming the hypothesis (FIG. 27).

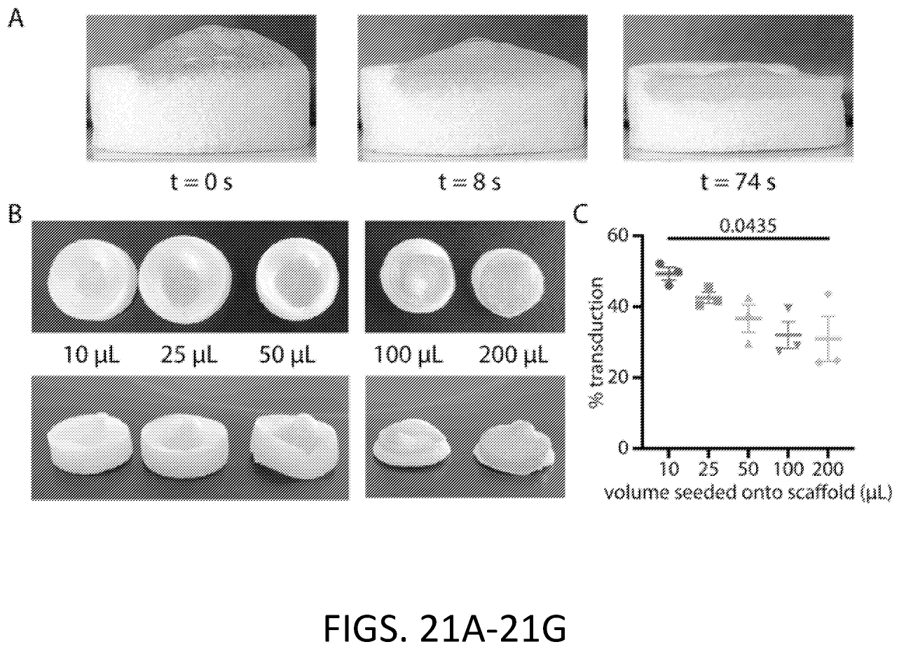

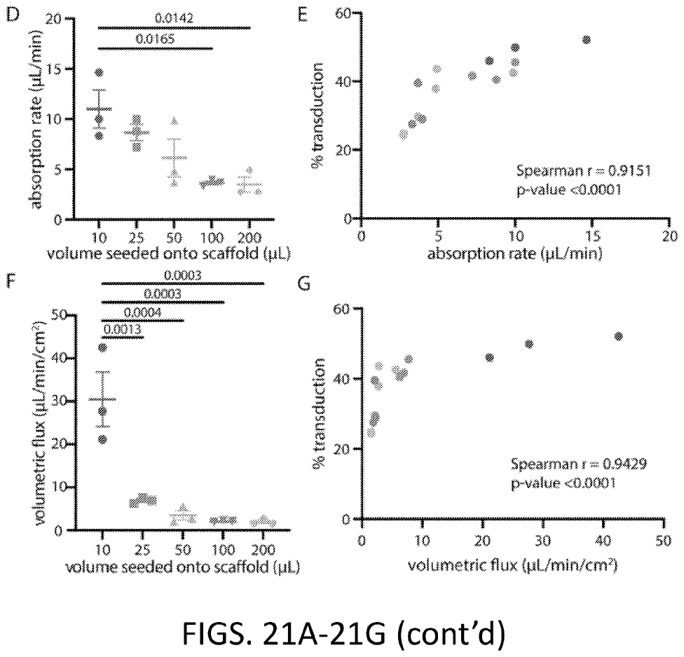

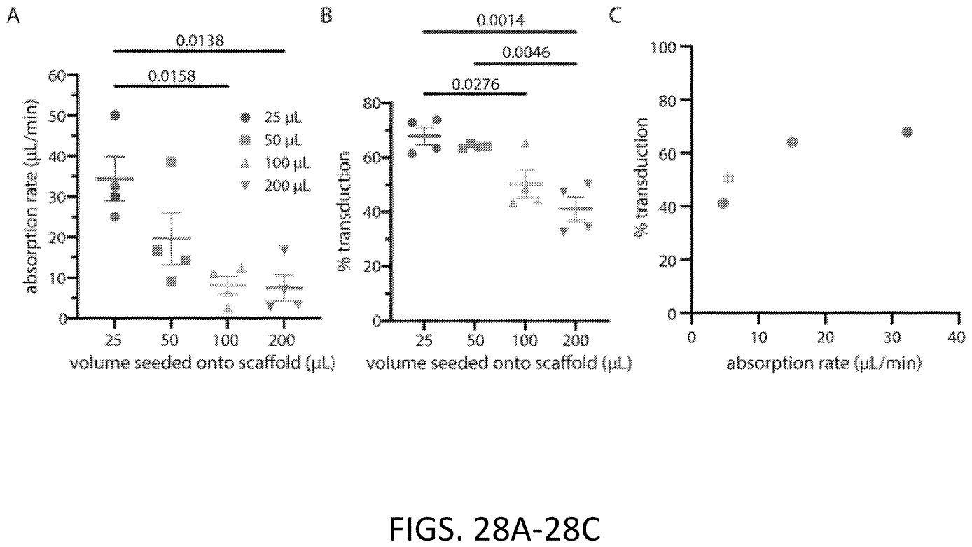

When performing the previous experiment, it was noted that larger volumes needed significantly more time to absorb into the scaffold (FIG. 21A, FIG. 28). It was wondered whether solution volume, and thereby absorption rate, could impact Drydux transduction. To explore this possibility, transduction of primary human PBMCs by retrovirus was evaluated at a constant concentration with different solution volume (10 μL, 25 μL, 50 μL, 100 μL, or 200 μL) (FIG. 21B). At the same time, the speed of liquid absorption into the scaffolds was measured by filming the absorption process and measuring the time it took for the volume to completely absorb into the scaffold. There was a clear trend in transduction efficiency, with smaller seed volumes producing significantly higher transduction efficiencies (FIG. 21C). In addition, there was a clear trend in absorption rate, with smaller seed volumes translating to faster absorption rates (FIG. 21D). Fitting a Spearman correlation to the data, a strong and significant correlation (p<0.0001) was discovered between the absorption rate and transduction efficiency of the scaffolds (FIG. 21E). Since different seed volumes had different absorption areas, determined as the area of the scaffold wetted by the droplet when viewing the top of the scaffold (FIG. 21B), the volumetric flux was calculated by dividing the absorption rate by the absorption area (FIG. 21F). A strong and significant Spearman correlation (p<0.0001) was discovered between volumetric flux and transduction (FIG. 21G).

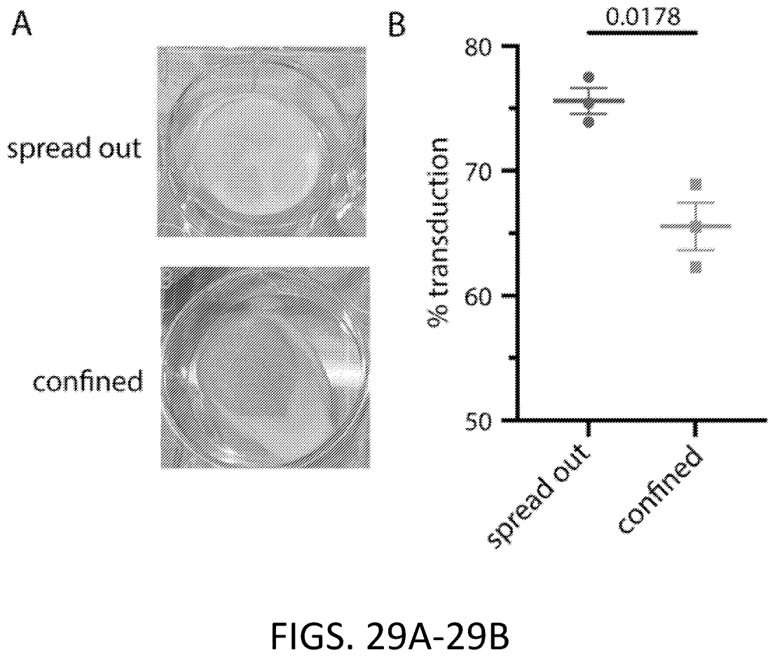

From these results, it was concluded that smaller seed volumes absorb into the scaffold faster, leading to increased volumetric flux and higher transduction efficiencies. From this, it stands to reason that spreading the cell-virus solution over a larger surface area would lead to faster absorption and increased transduction. To test this hypothesis, scaffolds with cross sectional areas of ˜8.12 cm² were created using 6-well plates and seeded solutions containing 4,000 cells/μL primary human PBMCs and 16,000 particles/μL retrovirus particles (MOI=4) onto the scaffold by either spreading the volume around the entire surface area or by seeding the volume in a single location (FIG. 29A). Liquid spread across the whole surface of the scaffold was absorbed faster and produced significantly higher transduction efficiencies than the same volume added in a single location of the scaffold (FIG. 29B). These results confirm that scaffold surface area can be used to control the absorption rate of the cell-virus solution to impact Drydux transduction.

### Example 12

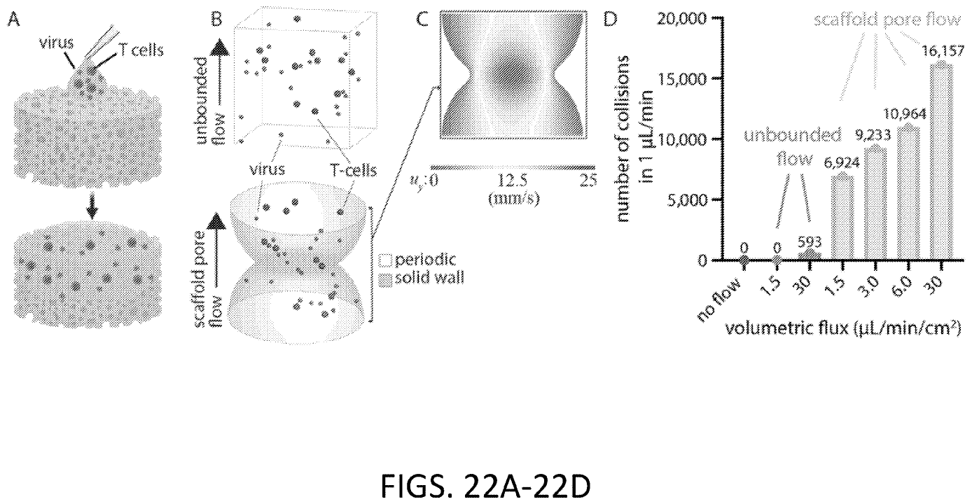

Computational model confirms porous structure is critical to transduction efficiency. The above results suggest that liquid flow through the scaffold during absorption governs Drydux transduction. To better understand the possible mechanism behind this observation, fluid flow through the scaffold was computationally simulated. It was hypothesized that liquid flow through the scaffold must increase the number of cell-virus collisions and that higher flow rates lead to a higher probability of collisions, thereby improving transduction. Scaffold flow was simulated in Ansys Fluent v21 using the Discrete Particle Method and Computational Fluid Dynamics. Viruses (radius=5×10⁻⁸ m) and cells (radius=3.5×10⁻⁶ m) were modeled as hard spheres flowing under three scenarios-stationary fluid, uniform unbounded flow (FIG. 22A) and flow through a scaffold pore (FIG. 22B). In accordance with previous data collected on pore geometry, the scaffold pore geometry was modeled as overlapping and interconnected spheres of radius 7.5×10⁻⁵ m that are spaced 1.3×10⁻⁴ m apart center-to-center (FIG. 22B). Periodic boundaries were applied to a representative elementary volume of the geometry to approximate the numerous pores present in the scaffold. The volumetric flux of the flow was varied (1.5, 3.0, 6.0, and 30.0 μL/min/cm²) to represent the experimental volumetric fluxes of different seed volumes into the scaffold pores as reported in FIG. 21F. The flow solution for a time period of 60 s was computed by numerically solving the incompressible Navier-Stokes equations. The particle trajectories were tracked from an initially random distribution using a one-way coupling with the flow solution since the particles occupy less than 0.1% of the liquid by volume. The particle model includes drag forces on the cell and virus particles, Brownian diffusion, and lift force under shear. When stationary fluid was modeled, no collisions were observed between viruses and T cells. In addition, no collisions were observed in the case of unbounded flow for a volumetric flux of 1.5 μL/min/cm² and a small number of collisions were observed for a volumetric flux of 30 μL/min/cm². However, when modeling flow inside the scaffold pore, the flow velocity was predicted to increase by a factor of 4 inside of the constriction point (FIG. 22C) when compared to the widest section of the pore so that the conservation of mass is satisfied. The increased flow velocity inside the scaffold pore created over a twentyfold increase in the number of collisions between T cells and viruses when compared to the unbounded flow (FIG. 22D). Furthermore, the number of collisions inside the scaffold pore consistently increased with the volumetric flux. The results highlight the importance of the scaffold geometry in promoting interaction between T cells and viruses during the transduction process.

In these examples, experimental results further defined the mechanism behind Drydux transduction by delineating the impact of scaffold pore size, stiffness, viral concentration, seeding volume, and absorption rate on transduction of human primary T cells. Macroporous alginate scaffolds were synthesized with varying physical properties by changing the alginate concentration, calcium concentration, and freezing temperature. Within the explored ranges, it was found that pore size had some, but unpredictable impact on transduction, while stiffness did not have any impact on transduction. Diluting the virus reduced transduction efficiency. Surprisingly, reducing the volume seeded onto the scaffold, without changing concentration, significantly improved cell transduction. It was found that seeded volume and transduction efficiency are both well correlated to the absorption rate, defined as the time needed for the liquid droplet to fully absorb into the scaffold. Taken together, these data indicated that the rate of absorption for the cell-virus solution likely governs Drydux transduction, suggesting specific ways to optimize Drydux scaffolds in future studies.

### 5. MATERIALS AND METHODS

Fabrication of Drydux. A macroporous alginate scaffold (Drydux) was fabricated as reported previously. Briefly, 2% w/v ultrapure alginate (Pronova, MVG) was dissolved in sterile filtered DI water and stirred vigorously. Once alginate was completely dissolved, an equal volume of 0.4% calcium-D-gluconate was mixed with the alginate solution and stirred vigorously for 15 min. The resulting gel was then cast in 48 well plates (300 μl/well) and frozen at −20° C. overnight. The next day, cryogels were transferred to a lyophilizer. After 72 hours, scaffolds were removed and stored in a vacuum-sealed bag at 4° C. until further use. As would be understood by one of ordinary skill in the art based on the present disclosure w/v %=mass of solute (g)/volume of solution (mL)×100. (E.g., 20 mg (0.02 g) of alginate in 1 mL of solution=0.02 g/l mL=0.02=2% w/v.) In some embodiments, alginate gels can be made at a 2× concentration of the final concentration. For example, one solution with alginate at 2% w/v can be combined with equal amounts calcium at 0.4% w/v. The final solution concentrations are 1% alginate at 0.2% calcium.

To make macroporous scaffolds for subcutaneous implantation (Drydux+IL2), recombinant human IL-2 (PeproTech), at a concentration of 0.2 μg/mg of alginate, was incorporated within the scaffolds before crosslinking and cryogelation.

X-ray CT. To characterize the macroporosity of Drydux, an X-ray CT scan was performed on the scaffolds. Scanning was done in an Xradia Versa 510 using Zeiss Scout and Scan version 13 with exposure of 8 seconds, ×4 optical magnification, 2.6 μm pixel size, 1600 projections with no filters, 74 μA current, and 40 kV voltage. A cylindrical volume of 2.50×2 mm was scanned in the sample. These volumes were then used to calculate the sample porosity and pore dimensions. A smaller sub-volume of 10×1 mm was extracted to visualize and calculate connectivity. The CT data were analyzed using Dragonfly 2020.1 software (Object Research Systems, http://www.theobjects.com/dragonfly). To segment the samples, a training dataset was created manually for each sample using histogram thresholding and masking techniques. Once the training data were created, they were used to train a deep-learning image segmentation model called U-net. The resulting model was then used to segment the full samples into scaffolds and porosity. To calculate the connectivity among the pores, an open-source package (openPNM 2.8) was used.

Conventional CAR T cell manufacturing. CAR T cells were manufactured in accordance with Good Manufacturing Practices currently used to manufacture clinical-grade cell products for clinical trials at the University of North Carolina at Chapel Hill. Human PBMCs were isolated from buffy coat fractions (Gulf Coast Regional Blood Center) of healthy donors using Lymphoprep density separation (Accurate Chemical and Scientific Corporation). Freshly isolated PBMCs were activated on plates coated with 1 μg/ml of CD3 (Miltenyi Biotec, 130-093-387, clone OKT-3) and CD28 (BD Biosciences, 555725, clone CD28.2) agonistic monoclonal antibodies. Retroviral supernatants used for the cell transduction were supplied by collaborators. To transduce activated T cells with GFP-encoding or CD19.CAR-encoding or B7H3.CAR-encoding retrovirus, 24 well plates were coated with RetroNectin (Takara Bio). Two days after T cell activation, retroviral supernatant was spinoculated on RetroNectin-coated plates for 90 minutes at 2000 g followed by activated T cells spinoculation on retrovirus- and RetroNectin-coated plates for 10 minutes at 1000 g. Plates were incubated for 72 hours, and then CAR T cells were harvested and expanded in 10 ng/ml of IL-7 (PeproTech) and 5 ng/ml of IL-15 (PeproTech) supplemented complete media consisting of Click's Medium (Irvine Scientific) and RPMI 1640 (1:1 v/v), 10% Hyclone FBS (GE Healthcare), 2 mmol/L GlutaMax (Gibco) and penicillin (100 U/ml) (Gibco) and streptomycin (100 mg/ml) (Gibco). On days 12-14, cells were collected for in vitro and in vivo experiments.

Scaffold mediated CAR T cell manufacturing. To manufacture CAR T cells using Drydux scaffolds, human T cells were isolated from the buffy coat (Gulf Coast Regional Blood Center) and activated on plates using of CD3 (1 μg/ml, Miltenyi Biotec, clone OKT-3) and CD28 (1 μg/ml, BD Biosciences, clone CD28.2) agonistic monoclonal antibodies as described above. To prepare retroviral supernatant for transduction, GFP-encoding or CD19.CAR-encoding, or B7H3.CAR-encoding retroviral supernatants were concentrated tenfold using Amicon centrifugation (MWCO 100 kDa, Millipore) at 2500 g for 15-20 min. Finally, activated cells and concentrated retroviral supernatant (MOI 2) were mixed together in ˜100 μl volume and pipetted onto each dry macroporous scaffold. Control scaffolds were seeded with only activated cells. For in vivo studies, seeded scaffolds were incubated for at least 1 hr. in 5% CO₂ at 37° C. before implantation. Scaffolds were then implanted subcutaneously in tumor-bearing NSG mice on the same day of transduction. For in vitro studies, seeded scaffolds were cultured for 72 hr. in excess media+/− cytokines. After 3 days of culture, scaffolds were digested with 0.25M EDTA (calcium chelator), washed twice with excess PBS, and cells were isolated. Isolated cells were analyzed for the expression of GFP or B7H3.CAR by flow cytometry. More than 95% of cells were recovered and viable. The remaining cells were cultured in complete media supplemented with 10 ng/ml of IL-7 (PeproTech) and 5 ng/ml of IL-15 (PeproTech) for various in vitro experiments.

Cell lines and culture. Daudi cells were purchased from the American Type Culture Collection (ATCC) and transduced with a retroviral vector encoding FFluc. After transduction, cells were selected in puromycin (Sigma-Aldrich). Cells were maintained in RPMI 1640 (Gibco) supplemented with 10% FBS (Gibco), 2 mmol/L GlutaMax (Gibco) and penicillin (100 U/ml) (Gibco), and streptomycin (100 mg/ml) (Gibco) at 37° C. with 5% CO2.

Human ovarian cancer cell line SKOV-3 (Source: female), human NSCLC cell line A549 and human pancreatic (PDAC) tumor cell line Panc-1 (Source: male) was received from Dr. Dotti's lab. These cell lines were originally purchased from ATCC and then transduced with a retroviral vector encoding GFP and Firefly-Luciferase (GFP-FFluc) gene.

SKOV-3 cells were cultured in McCoy's medium (Corning) supplemented with 10% FBS, 2 mM GlutaMax and (100 unit/mL) Penicillin (Gibco) and streptomycin (Gibco). A549 cells were cultured in RPMI 1640 (Gibco) supplemented with 10% FBS and 2 mM GlutaMax. Penicillin (100 unit/mL) (Gibco) and streptomycin (100 μg/mL) (Gibco) was added to the cell culture media. Panc-1 cells were cultured in DMEM (GIBCO) supplemented with 10% FBS, 2 mM GlutaMax and (100 unit/mL) Penicillin (Gibco) and streptomycin (Gibco). All cells were maintained in humidified atmosphere containing 5% CO₂ at 37° C.

In vitro cytotoxicity. Approximately 1×10⁵ GFP expressing tumor cells (SKOV3, A549, or Panc-1) were seeded per well in a 24-well plate. CAR T cells manufactured using a conventional method or Drydux scaffold were normalized to the transduction efficiencies and co-cultured with tumor cells at different effector-to-target (1:1, 1:5, and 1:10 E: T) ratios without any exogenous cytokines. Non-transduced cells were used as a negative control. After 5 days of co-culture, cells were collected to measure residual tumor cells by flow cytometry. Dead cells were gated out by Zombie Aqua Dye (Biolegend) staining, tumor cells (SKOV3, A549, and panc-1) were identified by the GFP expression and T cells were identified by expression of CD3.

ELISA. CAR T cells were co-cultured with tumor cells (SKOV3, A549, and panc-1) at 1:1, 1:5, and 1:10 E:T ratios without any addition of exogenous cytokines. After 24 hours, the supernatant was collected and IL-2 and IFN-γ were quantified by specific ELISA kits (R&D Systems) following the manufacturer's instructions.

In vitro cell proliferation and release. To assess the proliferation of cells within Drydux (with and without exogenous cytokines) and Drydux+IL2, activated T cells were labeled with 1.5 mM carboxyfluorescein diacetate succinimidyl ester (CFSE; Invitrogen) and seeded on the scaffolds. After 3 days, cells were isolated to determine cell count and CFSE dilution was measured using flow cytometry.

To assess the cell release, scaffolds seeded with activated T cells were placed in a 40 μm transwell (Corning). Fresh media was placed in the bottom chamber in contact with the mesh. At predetermined time points, media in the bottom chamber was collected to count the released cells, and scaffolds were moved to a new well containing fresh media.

In vitro safety assessment. To assess the potential of retroviral leakage from the scaffold upon implantation, an in vitro experiment was planned to indirectly validate the safety following scaffold implantation. Drydux scaffolds loaded with only cells or cells and GFP-encoding retrovirus were placed on the transwell (pore size 0.4 μm) and human fibroblast cells were seeded in the bottom well. At predetermined time points, fibroblasts were collected and analyzed for GFP expression using flow cytometry.

In vivo studies. All procedures involving animals were approved by North Carolina State University's Institutional Animal Care and Use Committee (IACUC) and were performed in compliance with IACUC. All animals were purchased from Animal core facility at UNC Chapel Hill. Animals were maintained under pathogen free conditions with regular health monitoring. All treated animals were monitored for signs of discomfort and euthanized upon losing more than 15% of initial body weight or the development of hind-limb paresis or hunched posture, or reaching a humane endpoint based on tumor burden. For the lymphoma xenograft tumor model, 10-12-week-old, female, immune-compromised NSG mice (NOD.Cg-Prkdescid Il2rgtm1Wjl/SzJ) were infused with 1×10⁶ FFluc expressing Daudi cells intravenously. Four days after infusion, each mouse was subcutaneously implanted with two Drydux scaffolds each loaded with 1×10⁶ PBMCs and CD19.CAR-encoding gamma retrovirus in IL-2-supplemented media (N=5). In the control group (N=3), mice were implanted with two Drydux scaffolds seeded with 1×10⁶ PBMCs each (non-transduced) in IL-2-supplemented media. Mice infused with 2×10⁶ CD19.CAR T cells generated by the conventional method were used as a positive control (N=5). Tumor burden was monitored weekly using the Xenogen-IVIS Imaging System. For solid tumor models (lung ovarian and pancreatic), animals were imaged before initiation of the treatment (day 0) and animals without apparent tumor signal were excluded from the study. Animals were randomized so that groups were matched based on tumor bioluminescence before assignment to control or treatment groups. Animal death following treatment was included in the survival study. For the metastatic model of NSLC, 1×10⁶ FFluc-A549 tumor cells were injected intravenously via tail vein injection in 8-10-week-old NSG mice. Two weeks after tumor cell inoculation, each animal received conventionally produced B7H3.CAR T cells (2.5×10⁶ CAR T cells/animal) intravenously (N=4) or two Drydux+IL2 scaffolds loaded with activated PBMCs and B7H3.CAR-encoding retrovirus (total dose of 2.5×10⁶ CAR T cells/animal) implanted subcutaneously (N=4). Two Drydux+IL2 scaffolds seeded with only PBMCs (total 2.5×10⁶ T cells) were implanted as controls (N=4). Tumor burden was monitored every week using the IVIS imaging system. 34 days post-treatment, blood was collected via mandibular check bleeds to analyze the number and immunophenotypic composition of B7H3.CAR+ cells. At the end of the study (day 99), blood, spleen, and bone marrow of conventional and Drydux treated groups were collected and number and immunophenotypic composition of B7H3.CAR+ cells was determined. For the ovarian xenograft tumor model, 5×10⁵ FFluc-SKOV3 cells were suspended in 1:1 PBS: Matrigel and inoculated intraperitoneally into 6-8-weeks-old female NSG mice. After 14 days, treatment was initiated with i.v. infusion of B7H3.CAR T cells (2.5×10⁶ CAR T cells/animal) manufactured conventionally (N=5) or subcutaneous implantation of two Drydux+IL2 scaffolds loaded with activated PBMCs and B7H3.CAR-encoding retrovirus (total dose of 2.5×10⁶ CAR T cells/animal) (N=6). Two Drydux+IL2 scaffolds seeded with only PBMCs (total 2.5×10⁶ T cells) were implanted as controls (N=3). Tumor burden was monitored every week using the AMI Imaging System. Study was repeated two time with two different PBMC donors. At day 34 post-treatment, blood was collected via mandibular check bleeds to analyze the number and immunophenotypic composition of B7H3.CAR T cells. At day 126, blood, spleen, and bone marrow of surviving group was collected, and number of B7H3.CAR+ cells was determined. For the orthotopic PDAC tumor model, 2×10⁵ FFluc-Panc-1 tumor cells were suspended in 50 μl of 1:1 PBS: Matrigel and surgically implanted into the pancreas of 8-10-week-old female NSG mice. Briefly, an incision was performed in the left flank to expose the pancreas and tumor cells were injected using a 29-gauge needle into the tail of the pancreas. The wound was closed in two layers, with running 4-0 Vicryl, and polypropylene sutures. Twelve days after tumor cell inoculation, CAR T cell treatment was initiated. Two Drydux+IL2 scaffolds loaded with activated PBMCs and B7H3.CAR-encoding retrovirus (total dose of 2.5×10⁶ CAR T cells/animal) were implanted subcutaneously (N=5). Two scaffolds seeded with only PBMCs (total 2.5×10⁶ T cells) were implanted as controls (N=6). Conventionally manufactured B7H3.CAR T cells (2.5×10⁶ CAR T cells/animal) were injected intravenously as positive controls (N=6). Animals were monitored for weight loss and tumor burden was measured every week using an IVIS imaging system. At days 20 and 40 post-treatment, blood was collected via mandibular cheek bleeds and the number of circulating CAR T cells was determined. At day 123, blood, spleen, and bone marrow of surviving group was collected, and number and immunophenotypic composition of B7H3.CAR+ cells was determined.

Flow cytometry and antibodies. All samples were acquired on a BD LSRII using BD FACSDiva software, and a minimum of 10,000 events were acquired per sample. CountBright absolute counting beads (C36950, Thermo Fisher Scientific) were used for calculating the absolute number of cells. Samples were analyzed with FlowJo software (version 10.8.1).

TABLE 1
Flow cytometry antibodies.

| Antibody | clone | Source | Cat# | Identifier |
|---|---|---|---|---|
| Goat Anti-Mouse IgG- | | Jackson | 115-606-072 | RRID: AB_2338928 |
| AF647 (Anti-Fab) | | ImmunoResearch | | |
| (Polyclonal | | Labs | | |
| Recombinant Human B7- | | R&D systems | 1027-B3 | |
| H3-Fc Chimeric Protein | | | | |
| B7-H3-BV421 | 7-517 | BD Biosciences | 565829 | RRID: AB_2739369 |
| CD3 (APC-Cy7) | SK7 | BD Biosciences | 557832 | RRID: AB_396890 |
| CD3 (PE) | UCHT1 | Biolegend | 300456 | |
| CD45 (BV510) | HI30 | BD Biosciences | 563204 | RRID: AB_2738067 |
| CD4 (APC-Cy7) | RPA-T4 | BD Biosciences | 561839 | RRID: AB_396913 |
| CD8 (PerCP-Cy5.5) | SK1 | BD Biosciences | 565310 | RRID: AB_2687497 |
| CD45RA (PE) | HI100 | BD Biosciences | 555489 | RRID: AB_395880 |
| CCR7 (FITC) | 150503 | R&D systems | FAB197F-100 | |
| PD-1(FITC) | MIH4 | BD Biosciences | 561035 | RRID: AB_2159176 |
| PD-1 (PerCP-Cy5.5) | EH12.1 | BD Biosciences | 561273 | RRID: AB_10645786 |
| LAG-3 (PE) | T47-530 | BD Biosciences | 565617 | RRID: AB_2571727 |
| LAG-3 (BV421) | T47-530 | BD Biosciences | 565720 | RRID: AB_2744330 |
| TIM-3(BV421) | 7D3 | BD Biosciences | 565563 | RRID: AB_2744369 |

Quantification and statistical analysis. Unpaired one-tailed or two-tailed Student's t-test with Holm-Sidak correction for multiple comparison was used to compare and perform statistical analysis between two groups. For multiple comparisons, one way ANOVA with Tukey post hoc analysis was used. All the analysis was performed using graph pad prism software version 9.4.1.

Preparation of macroporous alginate scaffolds. Scaffolds were prepared as reported previously. A solution of ultrapure alginate (Pronova, MVG) in DI water was vigorously mixed with an equal volume of calcium-D-gluconate solution in deionized (DI) water for 15 min. Final alginate concentrations used ranged from 0.5% to 2% and final calcium-D-gluconate concentrations ranged from 0.1% to 0.3% The resulting mixture was cast 1 mL per well in a 24-well plate and frozen overnight. Freezing temperatures ranged from −20° C. to −80° C. All frozen scaffolds were lyophilized for 72 h. Scaffolds were stored at 4° C. until used.

Viral titer and MOI determination. Viral titer was determined by standard flow-cytometry assay. Serially diluted viral stocks were added to HEK293T cells. GFP expression was analyzed using flow cytometry 48 h later, and populations with 10-20% GFP⁺ cells were used to calculate viral titer. The following equation was used to calculate titer: titer (TU/mL)=(cell number used for infection*percentage of GFP⁺ cells)/(virus volume used for infection in each well*dilution fold). MOI was calculated as the ratio of transducing viral particles to number of activated T cells. MOI values of 0.25 to 4 were tested using 0.5×10⁶ activated T cells to determine which MOI would give a transduction efficiency around 60%. Varying volumes of GFP viral stock were concentrated and mixed with 0.5×10⁶ activated primary T cells and seeded on top of dry macroporous alginate scaffolds. Scaffolds were incubated in 1 mL complete cell culture media (45% Click's Medium (Irvine Scientific), 45% RPMI-1640, 10% HyClone fetal bovine serum (GE Healthcare), 2 mmol/L GlutaMax (Gibco), penicillin (100 units/mL), and streptomycin (100 mg/mL; Gibco)) supplemented with IL-7 (Peprotech, 5 ng/mL) and IL-15 (Peprotech, 10 ng/ml) for 72 h. After 72 h, scaffolds were dissolved with 1 mL of 0.25 M EDTA. Cells were isolated and washed twice with PBS before being analyzed for GFP expression using flow cytometry.

Scanning electron microscopy. Dry macroporous alginate scaffolds were coated with 70 nm AuPd (Au: 60%, Pd: 40%) for 5 min at 7 nm/min and analyzed on Hitachi SU-3900 variable pressure SEM. Pore sizes were quantified using ImageJ to analyze the SEM images with a minimum of 10 pores measured per scaffold.

Compression testing. Dry macroporous alginate scaffolds were compressed using Instron 5944. Scaffolds were compressed with a 50 N force at a ramp rate of 0.1 mm/s. Force (N) and displacement (mm) was recorded every 100 ms. Stress was calculated using the equation: force/cross-sectional area. Cross-sectional area was determined using ImageJ to analyze images of each scaffold. Strain was calculated using the equation: displacement/initial length. Initial length was determined using ImageJ to analyze images of each scaffold. The Young's modulus was calculated by determining the slope of the stress-strain curves in the linear regions before the point of inflection.

Drydux transduction of activated T cells. GFP retroviral supernatant (5×10⁶ TU/mL) was concentrated using Amicon centrifugation filters (MWCO 100 kDa, Milipore) at 1,500 g for 10 min in a swinging bucket rotor. Concentrated retrovirus (2×10⁶ TU in 100 μL) was mixed with 1×10⁶ activated primary T cells (MOI=2) suspended in 50 μL complete cell culture media and pipetted onto the top of the dry macroporous alginate scaffolds. Seeded scaffolds were incubated for 45 min, after which 1 mL of complete cell culture media supplemented with IL-7 (Peprotech, 5 ng/mL) and IL-15 (Peprotech, 10 ng/mL) was added to each scaffold. After 72 h of incubation, scaffolds were dissolved with 1 mL of 0.25 M EDTA. Cells were isolated and washed twice with PBS before being analyzed for GFP expression using flow cytometry.

Absorption rate and volumetric flux. Different volumes of activated T cells and concentrated GFP-encoding retroviral supernatant were mixed, keeping a constant MOI of 2. Seeding of this mixture onto the scaffolds was filmed. The absorption rate was calculated as the liquid volume divided by the time it took for the entire droplet to absorb into the scaffold based on there being no liquid visible on top of the scaffold. Volumetric flux was calculated by dividing the absorption rate by the area of the scaffold wetted by the droplet when viewed from the top of the scaffold.

Cell lines. Peripheral blood mononuclear cells were isolated from a buffy coat (Gulf Coast Regional Blood Center) using Lymphoprep medium (Accurate Chemical and Scientific Corporation) and frozen in freeze media (50% HyClone fetal bovine serum (GE Healthcare), 40% RPMI-1640, 10% DMSO (Sigma)) until needed. Cells were thawed, resuspended in 9 mL complete media, and centrifuged at 400 g for 5 min to remove DMSO. Cells were activated on plates coated with 1 μg/mL of CD3 (Miltenyi Biotec, 130-093-387, clone OKT-3) and CD28 (BD Biosciences, 555725, clone CD28.2) agonistic monoclonal antibodies. GFP encoded retrovirus was prepared according to previously reported methods. All cells were maintained at 37° C. with 5% CO₂ and 95% humidity.

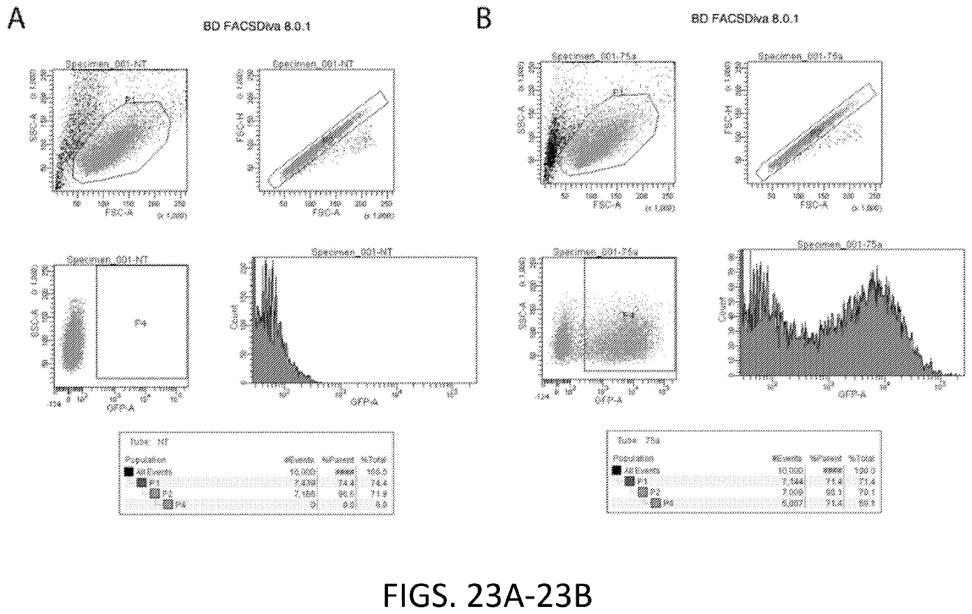

Flow cytometry. All samples were analyzed using BD LSRII with a minimum of 10,000 events acquired per sample. Cells were gated on viable cells, FSC singlets, and GFP positive cells (FIGS. 23A-23B). BD FACS Diva 8.0.1 software was used for analysis.

Statistical analysis and Spearman correlation. All statistical analysis was done using one-way ANOVA or two-way ANOVA with Tukey correction or unpaired t-test with Welch's correction using Graph Pad Prism 9. The specific test used and precise p-values are noted in individual figures. Spearman correlations were calculated using Graph Pad Prism 9 with r-values and p-values noted in individual figures. * indicates p<0.0001 with all other p-values indicated on plot.

What is claimed is:

1. An implantable macroporous scaffold comprising:
a crosslinked biopolymer matrix comprising an average pore size ranging from about 10 μm to about 500 μm, and a stiffness ranging from about 1 kPa to about 1000 kPa, wherein the stiffness of the matrix is compatible with the stiffness of a target tissue; and
a composition comprising a plurality of cells and a transduction agent;
wherein the scaffold facilitates transduction of the plurality of cells with the transduction agent.

2. The macroporous scaffold of claim 1, wherein the biopolymer matrix comprises at least one of alginate, Hyaluronic acid, collagen, fibrin, Poly Lactic-co-Glycolic Acid (PLGA), Polycaprolactone (PCL), gelatin, Polyethylene glycol (PEG), chitosan, cellulose, polyglutamic acid, fibrin, silk, agarose, dextran, polyacrylamide, polyvinyl alcohol, Poly(N-isopropylacrylamide), Poly(2-hydroxyethyl methacrylate), polyurethane, polyethyleneimine, Poly(methyl methacrylate, Poly(2-oxazoline), Polyphosphazenes, and any composites, derivatives, or combinations thereof.

3. The macroporous scaffold of claim 1 or claim 2, wherein the biopolymer matrix comprises alginate having a molecular weight from about 1 kDa to about 500 kDa.

4. The macroporous scaffold of any one of claims 1 to 3, wherein the biopolymer matrix comprises alginate having a G/M ratio from about 0.5 to about 5.0.

5. The macroporous scaffold of any one of claims 1 to 4, wherein the biopolymer matrix comprises alginate at a concentration ranging from about 0.1% to about 5.0% (w/v).

6. The macroporous scaffold of any one of claims 1 to 4, wherein the biopolymer matrix comprises alginate at a concentration ranging from about 0.5% to about 2.0% (w/v).

7. The macroporous scaffold of any one of claims 1 to 6, wherein the biopolymer matrix comprises alginate at a concentration ranging from about 0.5% to about 1.5% (w/v).

8. The macroporous scaffold of any one of claims 1 to 6, wherein the biopolymer matrix comprises alginate at a concentration ranging from about 0.5% to about 1.0% (w/v).

9. The macroporous scaffold of any one of claims 1 to 6, wherein the biopolymer matrix comprises alginate at a concentration ranging from about 1.0% to about 2.0% (w/v).

10. The macroporous scaffold of any one of claims 1 to 6, wherein the biopolymer matrix comprises alginate at a concentration ranging from about 1.0% to about 1.5% (w/v).

11. The macroporous scaffold of any one of claims 1 to 6, wherein the biopolymer matrix comprises alginate at a concentration ranging from about 1.5% to about 2.0% (w/v).

12. The macroporous scaffold of any one of claims 1 to 11, wherein the biopolymer matrix comprises calcium alginate having a calcium concentration ranging from about 0.1% to about 1.0% (w/v).

13. The macroporous scaffold of any one of claims 1 to 11, wherein the biopolymer matrix comprises calcium alginate having a calcium concentration ranging from about 0.1% to about 0.3% (w/v).

14. The macroporous scaffold of any one of claims 1 to 11, wherein the biopolymer matrix comprises calcium alginate having a calcium concentration ranging from about 0.1% to about 0.2% (w/v).

15. The macroporous scaffold of any one of claims 1 to 11, wherein the biopolymer matrix comprises calcium alginate having a calcium concentration ranging from about 0.2% to about 0.3% (w/v).

16. The macroporous scaffold of any one of claims 1 to 15, wherein the biopolymer matrix is generated at a temperature ranging from about 0° C. to about −80° C.

17. The macroporous scaffold of any one of claims 1 to 16, wherein the biopolymer matrix exhibits a stiffness that is from about ±25%, about ±50%, about ±75%, about ±100%, about ±125%, about ±150%, about ±175%, about ±200%, about ±225%, or about ±250% of the stiffness of the target tissue.

18. The macroporous scaffold of any one of claims 1 to 17, wherein the scaffold comprises at least one biological agent.

19. The macroporous scaffold of claim 18, wherein the at least one biological agent is a small molecule.

20. The macroporous scaffold of claim 19, wherein the small molecule is selected from the group consisting of a TLR agonist, a checkpoint inhibitor, an IDO inhibitor, a MEK inhibitor, an HDAC inhibitor, a PI3K inhibitor, an immunomodulatory drug, a JAK kinase inhibitor, and an mTOR inhibitor.

21. The macroporous scaffold of claim 18, wherein the at least one biological agent is a protein, peptide, or polypeptide.

22. The macroporous scaffold of claim 21, wherein the protein, peptide, or polypeptide is selected from the group consisting of a cytokine, an antibody, and a growth factor.

23. The macroporous scaffold of claim 22, wherein the cytokine comprises at least one of IL-2, IL-15, IL-7, IL-23, TNF-α, and/or IFN-γ.

24. The macroporous scaffold of any one of claims 1 to 23, wherein the plurality of cells comprise one or more immune cells.

25. The macroporous scaffold of claim 24, wherein the one or more immune cells are selected from the group consisting of a T cell, a B cell, a natural killer (NK) cell, an NK T cell, a macrophage, a dendritic cell, a tumor infiltrating lymphocyte (TIL), a tumor infiltrating NK cell (TINK), and a marrow infiltrating lymphocyte (MIL).

26. The macroporous scaffold of claim 24, wherein the one or more immune cells are activated.

27. The macroporous scaffold of any one of claims 1 to 26, wherein the plurality of cells are obtained from cell culture.

28. The macroporous scaffold of any one of claims 1 to 27, wherein the plurality of cells are obtained from a donor.

29. The macroporous scaffold of any one of claims 1 to 28, wherein the transduction agent comprises a viral vector.

30. The macroporous scaffold of claim 29, wherein the viral vector is selected from the group consisting of a lentivirus, a retrovirus, an adenovirus, an adeno-associated virus, a cocal virus, and a baculovirus.

31. The macroporous scaffold of claims 1 to 30, wherein the transduction agent comprises a virus-like particle, a cell-mimicking particle, a transposon, an exosome, a nanoparticle, a micelle, and a liposome.

32. The macroporous scaffold of any one of claims 1 to 22, wherein the transduction agent comprises a nucleic acid cargo.

33. The macroporous scaffold of claim 32, wherein the nucleic acid cargo comprises siRNA, tasiRNA, lncRNA, shRNA, mRNA, gRNA, miRNA, and/or viral RNA.

34. The macroporous scaffold of claim 32, wherein the nucleic acid cargo comprises DNA that encodes a fusion protein, a chimeric antigen receptor (CAR), a therapeutic peptide or polypeptide, or a combination thereof.

35. The macroporous scaffold of any one of claims 1 to 34, wherein the scaffold is implanted within or adjacent to the target tissue.

36. The macroporous scaffold of any one of claims 1 to 35, wherein the target tissue is tumor tissue.

37. The macroporous scaffold of any one of claims 1 to 36, wherein the target tissue is solid tumor tissue.

38. The macroporous scaffold of any one of claims 1 to 37, wherein the target tissue comprises at least one of lung tissue, bone tissue, skin tissue, breast tissue, muscle tissue, nerve tissue, brain tissue, lymph tissue, prostate tissue, bladder tissue, stomach tissue, intestinal tissue, uterine tissue, ovarian tissue, liver tissue, adipose tissue, cartilaginous tissue, thyroid tissue, and/or pancreatic tissue.

39. The macroporous scaffold of any one of claims 1 to 38, wherein the scaffold facilitates the transduction of the plurality of cells with the transduction agent with a transduction efficiency of at least 50%.

40. The macroporous scaffold of any one of claims 1 to 39, wherein the biopolymer matrix comprises an average pore size ranging from about 50 μm to about 250 μm.

41. The macroporous scaffold of any one of claims 1 to 39, wherein the biopolymer matrix comprises an average pore size ranging from about 100 μm to about 200 μm.

42. The macroporous scaffold of any one of claims 1 to 39, wherein the biopolymer matrix comprises an average pore size ranging from about 50 μm to about 150 μm.

43. The macroporous scaffold of any one of claims 1 to 42, wherein the biopolymer matrix comprises a stiffness ranging from about 25 kPa to about 650 kPa.

44. The macroporous scaffold of any one of claims 1 to 42, wherein the biopolymer matrix comprises a stiffness ranging from about 0.2 kPa to about 65 kPa.

45. The macroporous scaffold of any one of claims 1 to 42, wherein the biopolymer matrix comprises a stiffness ranging from about 0.1 kPa to about 10 kPa.

46. A method of treating a subject, the method comprising:
implanting a macroporous scaffold within or adjacent to a target tissue, wherein the scaffold comprises:
a crosslinked biopolymer matrix comprising an average pore size ranging from about 10 μm to about 500 μm, and a stiffness ranging from about 1 kPa to about 1000 kPa, wherein the stiffness of the matrix is compatible with the stiffness of the target tissue; and
a composition comprising a plurality of cells and a transduction agent;
wherein the scaffold facilitates transduction of the plurality of cells with the transduction agent, and wherein the transduced cells treat the subject.

47. The method of claim 46, wherein the target tissue is tumor tissue.

48. The method of claim 46 or claim 47, wherein the target tissue is solid tumor tissue.

49. The method of any one of claims 46 to 48, wherein the target tissue comprises at least one of lung tissue, bone tissue, skin tissue, breast tissue, muscle tissue, nerve tissue, brain tissue, lymph tissue, prostate tissue, bladder tissue, stomach tissue, intestinal tissue, uterine tissue, ovarian tissue, liver tissue, adipose tissue, cartilaginous tissue, thyroid tissue, and/or pancreatic tissue.

50. The method of any one of claims 46 to 49, wherein the subject has been diagnosed with a disease or condition.

51. The method of claim 50, wherein the disease or condition comprises cancer.

52. The method of any one of claims 46 to 51, wherein the scaffold facilitates the transduction of the plurality of cells with the transduction agent with a transduction efficiency of at least 50%.

53. The method of any one of claims 46 to 52, wherein the biopolymer matrix comprises at least one of alginate, Hyaluronic acid, collagen, fibrin, Poly Lactic-co-Glycolic Acid (PLGA), Polycaprolactone (PCL), gelatin, Polyethylene glycol (PEG), chitosan, cellulose, polyglutamic acid, fibrin, silk, agarose, dextran, polyacrylamide, polyvinyl alcohol, Poly(N-isopropylacrylamide), Poly(2-hydroxyethyl methacrylate), polyurethane, polyethyleneimine, Poly(methyl methacrylate, Poly(2-oxazoline), Polyphosphazenes, and any composites, derivatives, or combinations thereof.

54. The method of any one of claims 46 to 53, wherein the biopolymer matrix comprises alginate having a molecular weight from about 1 kDa to about 500 kDa.

55. The method of any one of claims 46 to 54, wherein the biopolymer matrix comprises alginate having a G/M ratio from about 0.5 to about 5.0.

56. The method of any one of claims 46 to 55, wherein the biopolymer matrix comprises alginate at a concentration ranging from about 0.1% to about 5.0%.

57. The method of any one of claims 31 to 56, wherein the biopolymer matrix comprises calcium alginate having a calcium concentration ranging from about 0.1% to about 1.0%.

58. The method of any one of claims 31 to 57, wherein the biopolymer matrix is generated at a temperature ranging from about −20° C. to about −80° C.

59. The method of any one of claims 46 to 58, wherein the biopolymer matrix exhibits a stiffness that is from about ±25%, about ±50%, about ±75%, about ±100%, about ±125%, about ±150%, about ±175%, about ±200%, about ±225%, or about ±250% of the stiffness of the target tissue.

60. The method of any one of claims 46 to 59, wherein the scaffold comprises at least one biological agent.

61. The method of claim 60, wherein the at least one biological agent is a small molecule.

62. The method of claim 61, wherein the small molecule is selected from the group consisting of a TLR agonist, a checkpoint inhibitor, an IDO inhibitor, a MEK inhibitor, an HDAC inhibitor, a PI3K inhibitor, an immunomodulatory drug, a JAK kinase inhibitor, and an mTOR inhibitor.

63. The method of claim 60, wherein the at least one biological agent is a protein, peptide, or polypeptide.

64. The method of claim 63, wherein the protein, peptide, or polypeptide is selected from the group consisting of a cytokine, an antibody, and a growth factor.

65. The method of claim 64, wherein the cytokine comprises at least one of IL-2, IL-15, IL-7, IL-23, TNF-α, and/or IFN-γ.

66. The method of any one of claims 46 to 65, wherein the plurality of cells comprise one or more immune cells.

67. The method of claim 66, wherein the one or more immune cells are selected from the group consisting of a T cell, a B cell, a natural killer (NK) cell, an NK T cell, a macrophage, a dendritic cell, a tumor infiltrating lymphocyte (TIL), a tumor infiltrating NK cell (TINK), and a marrow infiltrating lymphocyte (MIL).

68. The method of claim 67, wherein the one or more immune cells are activated.

69. The method of any one of claims 46 to 68, wherein the plurality of cells are obtained from cell culture.

70. The method of any one of claims 46 to 69, wherein the plurality of cells are obtained from a donor.

71. The method of any one of claims 46 to 70, wherein the transduction agent comprises a viral vector.

72. The method of claim 71, wherein the viral vector is selected from the group consisting of a lentivirus, a retrovirus, an adenovirus, an adeno-associated virus, a cocal virus, and a baculovirus.

73. The method of any one of claims 46 to 72, wherein the transduction agent comprises a virus-like particle, a cell-mimicking particle, a transposon, an exosome, a nanoparticle, a micelle, and a liposome.

74. The method of any one of claims 46 to 73, wherein the transduction agent comprises a nucleic acid cargo.

75. The method of claim 74, wherein the nucleic acid cargo comprises siRNA, tasiRNA, lncRNA, shRNA, mRNA, gRNA, miRNA, and/or viral RNA.

76. The method of claim 74, wherein the nucleic acid cargo comprises DNA that encodes a fusion protein, a chimeric antigen receptor (CAR), a therapeutic peptide or polypeptide, or a combination thereof.
